(19) United States
(12) Patent Application Publication
(10) Pub. No.: US 2024/0417764 A1
(43) Pub. Date: Dec. 19, 2024

(54) METHODS FOR THE PRODUCTION OF TRYPTOPHANS, TRYPTAMINES, INTERMEDIATES, SIDE PRODUCTS AND DERIVATIVES

(71) Applicant: Miami University, Oxford, OH (US)

(72) Inventors: John Andrew Jones, Liberty Township, OH (US); William Johnathan Gibbons, JR., Oxford, OH (US); Jessica Flower, Algonquin, IL (US); Alexandra Adams, Liberty Township, OH (US)

(21) Appl. No.: 18/706,982

(22) PCT Filed: Nov. 4, 2022
(86) PCT No.: PCT/US2022/079321

§ 371 Date: May 2, 2024

Related U.S. Application Data
(60) Provisional application No. 63/263,616, filed on Nov. 5, 2021.
(60) Provisional application No. 63/263,623, filed on Nov. 5, 2021.

(51) Int. Cl. C12P 13/22; C12N 9/02; C12N 9/10; C12N 9/12; C12N 9/88; C12P 17/10

(52) U.S. Cl. CPC C12P 13/227; C12N 9/0083; C12N 9/1007; C12N 9/1205; C12N 9/88; C12P 17/10; C12Y 401/01

(57) ABSTRACT

Provided are methods, prokaryotic host cells, expression vectors, and kits for the production of a tryptophan, a tryptamine, or an intermediate or a side product thereof, or a derivative thereof. In some embodiments, the tryptophan, tryptamine, intermediate or side product is a non-naturally occurring derivative. In some embodiments, the tryptamine is a psilocybin derivative. In certain embodiments, the prokaryotic host cell is selected from the group consisting of Escherichia coli, Corynebacterium glutamicum, Vibrio natriegens, Bacillus subtilis, Bacillus megaterium, Escherichia coli Nissle 1917, Clostridium acetobutlyicum, Streptomyces coelicolor, Lactococcus lactis, Pseudomonas putida, Streptomyces clavuligerus, and Streptomyces venezuelae.

### FIELD

The general inventive concepts relate to the field of medical therapeutics and more particularly to methods for the production of tryptophans, tryptamines, intermediates, side products and derivatives.

### CROSS-REFERENCE TO RELATED APPLICATIONS

The instant application is entitled to priority under 35 U.S.C. § 119(e) to U.S. Provisional Application No. 63/263,616, filed Nov. 5, 2021, and U.S. Provisional Application No. 63/263,623, filed Nov. 5, 2021, each of which is hereby incorporated by reference in its entirety.

### SEQUENCE LISTING

The contents of the electronic sequence listing (315691-00044.xml; Size: 92,200 bytes; and Date of Creation: Nov. 4, 2022) is herein incorporated by reference in its entirety.

### BACKGROUND

Natural tryptamines, such as psilocybin and N,N-dimethyltryptamine (DMT), have structural similarities to the neurotransmitter serotonin and impart vivid hallucinations to humans upon ingestion. Psilocybin, despite being currently listed as a Schedule I drug in the United States, is currently undergoing promising clinical trials to investigate its therapeutic efficacy for a number of indications, including post-traumatic stress disorder (PTSD) (Gluff et al., 2017; Johnson and Griffiths, 2017), depression (Carhart-Harris et al., 2017, 2016), obsessive compulsive disorder (OCD) (Matsushima et al., 2009), and addiction (Garcia-Romeu et al., 2014). With the ongoing success of psilocybin clinical trials, non-natural psilocybin derivatives are also of interest as potential new drugs. This necessitates the synthesis and study of various psilocybin derivatives, with substitutions on various locations of the chemical structure, to create a larger drug candidate pool. To date, most of these derivatives have been produced via traditional synthetic organic synthesis methods (Sherwood et al., 2020). However, enzyme-catalyzed synthesis routes, both in vitro and in vivo, for the production of psilocybin and psilocybin derivatives are also being actively explored (Adams et al., 2022, 2019; Blei et al., 2018; Milne et al., 2020a).

Along with the development of bio-based methods to produce a larger drug candidate pool comes the study of the structure-activity relationship (SAR) of those psilocybin derivatives (Cao et al., 2022; Glennon et al., 1980; Klein et al., 2021; Kline et al., 1982; Lyon et al., 1988; Macor et al., 1992; May et al., 2003; Repke et al., 1985). Currently, there is a dearth of knowledge relating to the pharmacological properties of these compounds on the serotonin receptor family as well as their impact on animal behavior and disease efficacy. Producing a wider variety of psilocybin derivatives in sufficient quantity to enable preliminary testing is crucial to increasing our SAR knowledge for this powerful group of drug candidate compounds.

To this end, it is important to leverage new and existing bio-enzymatic methods of tryptophan and tryptamine derivative production to construct more efficient and robust routes for the production of psilocybin derivatives. Significant effort towards identifying, evaluating, and improving tryptophan synthase and decarboxylase enzymes has been undertaken by a range of researchers (Phillips, 2004; Romney et al., 2017; Watkins-Dulaney et al., 2021). Tryptophan synthases sourced from various organisms including Neurospora crassa (HALL et al., 1962; Saito and Rilling, 2006), Escherichia coli (Crowley et al., 2012; Held and Smith, 1970; Miles and Phillips, 1985; Wilcox, 1974; Xu et al., 2021, 2020), Salmonella typhimurium (Cash et al., 2004; Francis et al., 2017; Miles and Phillips, 1985; Phillips et al., 1995; Sloan and Phillips, 1992; Welch and Phillips, 1999), Salmonella enterica (Goss and Newill, 2006; Hoffarth et al., 2021; Smith et al., 2014), Pyrococcus furiosus (Boville et al., 2018b; Buller et al., 2015; Herger et al., 2016; Murciano-Calles et al., 2016), Psilocybe cubensis (Blei et al., 2018), and Thermotoga maritima (Boville et al., 2018a) have generally been characterized to have activity on a broad range of substrates and have been shown to be highly engineerable with recent studies enabling standalone activity from the tryptophan synthase 3-subunit (Buller et al., 2015; Murciano-Calles et al., 2016) and activity under mild reaction conditions (Boville et al., 2018a; Rix et al., 2020).

Recent efforts focusing on characterizing tryptophan decarboxylase (TDC) activity have focused primarily on the use of TDCs sourced from Catharanthus roseus and the more recently discovered, highly active, TDC from Ruminococcus gnavus. C. roseus TDC has been used to produce halogenated tryptamines in plants (Runguphan et al., 2010) and more than 20 tryptamine analogs have been produced utilizing engineered TrpBs from P. furiosus and the R. gnavus TDC (McDonald et al., 2019). Native E. coli TrpS, the combination of both α- and β-subunits, was used previously, along with the psilocybin pathway enzymes PsiD, PsiK, and PsiM to produce psilocybin from 4-hydroxyindole in E. coli (Adams et al., 2019; Gibbons et al., 2021). The ability of this pathway to utilize diverse substrates has not previously been explored.

There remains a need for methods for the production of non-naturally occurring tryptophans, tryptamines, intermediates, side products and derivatives.

### SUMMARY

Provided is a method for the production of a tryptophan, a tryptamine, or an intermediate, or a side product thereof, or a derivative thereof, comprising contacting a first prokaryotic host cell with one or more expression vectors, wherein each expression vector comprises a gene selected from the group consisting of psiH, CPR, and combinations thereof, contacting a second prokaryotic host cell with one or more expression vectors, wherein each expression vector comprises a gene selected from the group consisting of psiD, psiK, psiM, and combinations thereof, and co-culturing the first prokaryotic host cell with the second prokaryotic host cell.

In some embodiments, the tryptophan, tryptamine, intermediate or side product is a non-naturally occurring derivative.

In some embodiments, the tryptamine is a non-naturally occurring tryptamine derivative. In further embodiments, the non-naturally occurring tryptamine derivative is a substituted 4-hydroxytryptamine.

In some embodiments, the tryptamine is a psilocybin derivative.

In certain embodiments, the first prokaryotic host cell is selected from the group consisting of Escherichia coli, Corynebacterium glutamicum, Vibrio natriegens, Bacillus subtilis, Bacillus megaterium, Escherichia coli Nissle 1917, Clostridium acetobutlyicum, Streptomyces coelicolor, Lactococcus lactis, Pseudomonas putida, Streptomyces clavuligerus, and Streptomyces venezuelae.

In certain embodiments, the second prokaryotic host cell is selected from the group consisting of Escherichia coli, Corynebacterium glutamicum, Vibrio natriegens, Bacillus subtilis, Bacillus megaterium, Escherichia coli Nissle 1917, Clostridium acetobutlyicum, Streptomyces coelicolor, Lactococcus lactis, Pseudomonas putida, Streptomyces clavuligerus, and Streptomyces venezuelae.

In some embodiments, the intermediate or side product of psilocybin is norbaeocystin, baeocystin, 4-hydroxytryptophan, 4-hydroxytryptamine, aeruginascin, psilocin, norpsilocin, or 4-hydroxy-N,N,N-trimethyltryptamine (4-OH-TMT). In some embodiments the intermediate of psilocybin is norbaeocystin, baeocystin, 4-hydroxytryptophan, or 4-hydroxytryptamine. In some embodiments, the side product of psilocybin is aeruginascin, psilocin, norpsilocin, or 4-hydroxy-N,N,N-trimethyltryptamine (4-OH-TMT).

Also provided is a method for the production of a tryptophan, a tryptamine, or an intermediate, a derivative, or a side product thereof comprising: contacting a prokaryotic host cell with one or more expression vectors, wherein each expression vector comprises a gene selected from the group consisting of psiH, CPR, psiD, psiK, psiM, and combinations thereof.

In some embodiments, the tryptophan, tryptamine, intermediate or side product is a non-naturally occurring derivative.

In some embodiments, the tryptamine is a non-naturally occurring tryptamine derivative. In further embodiments, the non-naturally occurring tryptamine derivative is a substituted 4-hydroxytryptamine.

In some embodiments, the tryptamine is a psilocybin derivative.

Also provided is a recombinant prokaryotic cell comprising one or more expression vectors, wherein each expression vector comprises a gene selected from the group consisting of psiH, CPR and combinations thereof.

Provided is an expression vector comprising a psiH gene, and a CPR gene. Also provided is an expression vector comprising a psiH gene, and a CPR gene all under control of a single promoter in operon configuration. Further provided is an expression vector comprising a psiH gene, and a CPR gene, wherein each gene is under control of a separate promoter in pseudooperon or monocistronic configuration.

Also provided is a transfection kit comprising an expression vector as described herein.

Provided is a method for the production of a tryptophan, a tryptamine, or an intermediate, a derivative, or a side product thereof comprising: contacting a prokaryotic host cell with one or more expression vectors, wherein each expression vector comprises a gene selected from the group consisting of psiH, CPR, and combinations thereof, and culturing the host cell.

In some embodiments, the tryptophan, tryptamine, intermediate or side product is a non-naturally occurring derivative.

In some embodiments, the tryptamine is a non-naturally occurring tryptamine derivative. In further embodiments, the non-naturally occurring tryptamine derivative is a substituted 4-hydroxytryptamine.

In some embodiments, the tryptamine is a psilocybin derivative.

In certain embodiments, the prokaryotic host cell is selected from the group consisting of Escherichia coli, Corynebacterium glutamicum, Vibrio natriegens, Bacillus subtilis, Bacillus megaterium, Escherichia coli Nissle 1917, Clostridium acetobutlyicum, Streptomyces coelicolor, Lactococcus lactis, Pseudomonas putida, Streptomyces clavuligerus, and Streptomyces venezuelae.

### DESCRIPTION OF THE FIGURES

FIG. 1 shows a reconstituted psilocybin biosynthesis pathway for the production of de novo psilocybin (from endogenous tryptophan; R=H) or psilocybin derivatives (from supplemented indole derivatives, dashed line; R≠H).

FIG. 2 shows psilocybin production using an E. coli co-culture approach. (1) Glucose is utilized by endogenous metabolism to produce the amino acid tryptophan. PsiD then acts on tryptophan to product tryptamine. (2) Tryptamine is transferred to JF06 where it is used as substrate for PsiH/PcCPR to produce 4-hydroxytryptamine. (3) 4-hydroxytryptamine is transferred back to the original pPsilo16 strain (pPsilo16 is also referred to as “pSilo16”) and converted to psilocybin by the action of PsiK and PsiM. (4) Psilocybin is excreted from the cell into the cell broth. An analogous process can enable psilocybin derivative biosynthesis, through the supplementation of indole derivatives to the culture media.

FIG. 3 shows twelve hydroxylase modules containing PsiH and either PcCPR or TcCPR. *Reaction products detected by LCMS analysis.

FIG. 4 shows verification of 4-hydroxytryptamine production by JF06. The top trace (black) represents JF06 supplemented with tryptamine, while the bottom trace (gray) represents an empty vector control supplemented with tryptamine. The 4-hydroxytryptamine product can be observed at 3.6 min in the top trace. The mass spectrum of 4-hydroxytryptamine indicating a m/z 177 is provided in the inset,

FIGS. 5A-5B show fermentation conditions optimization of JF06 hydroxylase module. (FIG. 5A) Fermentation temperature optimization, and (FIG. 5B) induction point optimization.

FIGS. 6A-6B show basic co-culture evaluation of JF06 with a PsiD expressing E. coli strain. (FIG. 6A) Biosynthesis pathway utilized in this study with the PsiD expression in one strain and PsiH/CPR in another. (FIG. 6B) 4-hydroxytryptamine production from a basic co-culture system under different supplement and inoculation ratio conditions.

FIG. 7 shows de novo psilocybin production across multiple inoculation ratios.

FIG. 8 shows verification of psilocybin production by the JF06:pSilo16 co-culture under de novo conditions. The top trace (black) represents the co-culture of JF06:pSilo16, while the bottom trace (gray) represents the psilocybin production strain, pSilo16, monoculture. The psilocybin product can be observed at 2.7 min in the top trace. The mass spectrum of psilocybin indicating a m/z 285 is provided in the inset.

FIG. 9 shows evaluation of inducer concentration on pathway performance.

FIG. 10 shows impact of additional iron in media on pathway performance. The base media contains 1.5 mg/L (9.9 uM) of FeSO₄ under all conditions.

FIGS. 11A-11C show evaluation of E. coli TrpB substrate flexibility with and without supplemental serine. (FIG. 11A) indole structure with substitution positions labeled 1-7, (FIG. 11B) Yield for production of tryptophan derivatives by native E. coli TrpB from indole derivatives without serine supplementation, and (FIG. 11C) Yield for production of tryptophan derivatives by native E. coli TrpB from indole derivatives with serine supplementation. n.t., not tested.

FIGS. 12A-12E show a summary of aminotryptophan production using native E. coli TrpS in vivo. Product specific LCMS extracted ion chromatograph shown in FIGS. 12B-12E, with no-indole controls shown in FIG. 12A.

FIGS. 13A-13J show a summary of bromotryptophan production using native E. coli TrpS in vivo. Product specific LCMS extracted ion chromatograph shown in FIGS. 13B-13E and 13G-13J, with no-indole controls shown in 13A and 13F, for the ⁷⁹Br and ⁸¹Br, respectively.

FIGS. 14A-14F show a summary of chlorotryptophan production using native E. coli TrpS in vivo. Product specific LCMS extracted ion chromatograph shown in FIGS. 14B-14F, with no-indole controls shown in FIG. 14A.

FIGS. 15A-15E show a summary of cyanotryptophan production using native E. coli TrpS in vivo. Product specific LCMS extracted ion chromatograph shown in FIGS. 15B-15E, with no-indole controls shown in FIG. 15A.

FIGS. 16A-16F show a summary of fluorotryptophan production using native E. coli TrpS in vivo. Product specific LCMS extracted ion chromatograph shown in FIGS. 16B-16F, with no-indole controls shown in FIG. 16A.

FIGS. 17A-17F show a summary of hydroxytryptophan production using native E. coli TrpS in vivo. Product specific LCMS extracted ion chromatograph shown in FIGS. 17B-17F, with no-indole controls shown in FIG. 17A.

FIGS. 18A-18E show a summary of iodotryptophan production using native E. coli TrpS in vivo. Product specific LCMS extracted ion chromatograph shown in FIGS. 18B-18E, with no-indole controls shown in FIG. 18A.

FIGS. 19A-19E show a summary of methoxytryptophan production using native E. coli TrpS in vivo. Product specific LCMS extracted ion chromatograph shown in FIGS. 19B-19E, with no-indole controls shown in FIG. 19A.

FIGS. 20A-20G show a summary of methyltryptophan production using native E. coli TrpS in vivo. Product specific LCMS extracted ion chromatograph shown in FIGS. 20B-20G, with no-indole controls shown in FIG. 20A.

FIGS. 21A-21E show a summary of nitrotryptophan production using native E. coli TrpS in vivo. Product specific LCMS extracted ion chromatograph shown in FIGS. 21B-21E, with no-indole controls shown in FIG. 21A.

FIGS. 22A-22B show Evaluation of P. cubensis PsiD substrate flexibility with and without transcriptional optimization. (FIG. 22A) Yield for production of tryptamine derivatives with PsiD expression controlled by consensus T7 promoter, and (FIG. 22B) Yield for production of tryptamine derivatives with PsiD expression controlled by best construct from transcriptionally varied library. n.t., not tested; XXX, tryptophan substrate not observed from TrpB study; ‘<1’, tryptamine product not observed with consensus T7 construct, but was observed using either optimized psilocybin or norbaeocystin production strains, pSilo16 and pNor, respectively.

FIG. 23 shows a PsiD transcriptional library screening for aminoindole derivatives. Error bars represent+/−1 standard deviation of biological duplicate cultures.

FIG. 24 shows a PsiD transcriptional library screening for bromoindole derivatives. Error bars represent+/−1 standard deviation of biological duplicate (6- and 7-bromo) or triplicate (4- and 5-bromo) cultures.

FIG. 25 shows a PsiD transcriptional library screening for chloroindole derivatives. Error bars represent+/−1 standard deviation of biological duplicate cultures.

FIG. 26 shows a PsiD transcriptional library screening for cyanoindole derivatives. Error bars represent+/−1 standard deviation of biological duplicate cultures.

FIG. 27 shows a PsiD transcriptional library screening for fluoroindole derivatives. Error bars represent+/−1 standard deviation of biological duplicate cultures.

FIG. 28 shows a PsiD transcriptional library screening for hydroxyindole derivatives. Error bars represent+/−1 standard deviation of biological duplicate cultures.

FIG. 29 shows a PsiD transcriptional library screening for iodoindole derivatives. Error bars represent+/−1 standard deviation of biological duplicate cultures.

FIG. 30 shows a PsiD transcriptional library screening for methoxyindole derivatives. Error bars represent+/−1 standard deviation of biological duplicate cultures.

FIG. 31 shows a PsiD transcriptional library screening for methylindole derivatives. Error bars represent+/−1 standard deviation of biological duplicate cultures.

FIG. 32 shows a PsiD transcriptional library screening for nitroindole derivatives. Error bars represent+/−1 standard deviation of biological duplicate cultures.

FIG. 33 shows a summary of aminotryptamine production using P. cubensis PsiD in vivo. Product specific LCMS extracted ion chromatograph shown in black, with no-indole controls shown in gray.

FIGS. 34A-34H show a summary of bromotryptamine production using P. cubensis PsiD in vivo. Product specific LCMS extracted ion chromatograph shown in black, with no-indole controls shown in gray.

FIGS. 35A-35D show a summary of chlorotryptamine production using P. cubensis PsiD in vivo. Product specific LCMS extracted ion chromatograph shown in black, with no-indole controls shown in gray.

FIG. 36 shows a summary of cyanotryptamine production using P. cubensis PsiD in vivo. Product specific LCMS extracted ion chromatograph shown in black, with no-indole controls shown in gray.

FIGS. 37A-37D show a summary of fluorotryptamine production using P. cubensis PsiD in vivo. Product specific LCMS extracted ion chromatograph shown in black, with no-indole controls shown in gray.

FIGS. 38A-38D show a summary of hydroxytryptamine production using P. cubensis PsiD in vivo. Product specific LCMS extracted ion chromatograph shown in black, with no-indole controls shown in gray.

FIGS. 39A-39C show a summary of iodotryptamine production using P. cubensis PsiD in vivo. Product specific LCMS extracted ion chromatograph shown in black, with no-indole controls shown in gray.

FIGS. 40A-40D show a summary of methoxytryptamine production using P. cubensis PsiD in vivo. Product specific LCMS extracted ion chromatograph shown in black, with no-indole controls shown in gray.

FIGS. 41A-41D show a summary of methyltryptamine production using P. cubensis PsiD in vivo. Product specific LCMS extracted ion chromatograph shown in black, with no-indole controls shown in gray.

FIG. 42 shows a summary of nitrotryptamine production using P. cubensis PsiD in vivo. Product specific LCMS extracted ion chromatograph shown in black, with no-indole controls shown in gray.

FIG. 43 shows LCMS extracted ion chromatograph showing 5-fluoropsilocybin production using an E. coli co-culture approach (black). No indole controls are shown in gray.

FIGS. 44A-44G show a summary of 6-position psilocybin derivative production using an E. coli co-culture approach. Product specific LCMS extracted ion chromatograph shown in black, with no-indole controls shown in gray.

FIGS. 45A-45G show a summary of 7-position psilocybin derivative production using an E. coli co-culture approach. Product specific LCMS extracted ion chromatograph shown in black, with no-indole controls shown in gray.

FIG. 46 shows LCMS extracted ion chromatograph showing 5-fluorobaeocystin production using an E. coli co-culture approach (black). No indole controls are shown in gray.

FIGS. 47A-47G show a summary of 6-position baeocystin derivative production using an E. coli co-culture approach. Product specific LCMS extracted ion chromatograph shown in black, with no-indole controls shown in gray.

FIGS. 48A-48G show a summary of 7-position baeocystin derivative production using an E. coli co-culture approach. Product specific LCMS extracted ion chromatograph shown in black, with no-indole controls shown in gray.

FIG. 49 shows LCMS extracted ion chromatograph showing 5-fluoronorbaeocystin production using an E. coli co-culture approach (black). No indole controls are shown in gray.

FIGS. 50A-50G show a summary of 6-position norbaeocystin derivative production using an E. coli co-culture approach. Product specific LCMS extracted ion chromatograph shown in black, with no-indole controls shown in gray.

FIGS. 51A-51G show a summary of 7-position norbaeocystin derivative production using an E. coli co-culture approach. Product specific LCMS extracted ion chromatograph shown in black, with no-indole controls shown in gray.

FIG. 52 shows a summary of substrate flexibility for psilocybin production pathway in E. coli. ‘Yes’ indicates derivative product observed by HPLC or LCMS analysis. ‘No’ indicates derivative product not observed by HPLC or LCMS analysis. n.t., not tested; N/A, resulting product can not be formed due to blocked 4-position; Yes*, intermediate product not observed, but assumed present due to observation of final product.

### DETAILED DESCRIPTION

While the general inventive concepts are susceptible of embodiment in many forms, there are shown in the drawings, and will be described herein in detail, specific embodiments thereof with the understanding that the present disclosure is to be considered an exemplification of the principles of the general inventive concepts. Accordingly, the general inventive concepts are not intended to be limited to the specific embodiments illustrated herein.

It is also to be understood that the terminology used herein is for the purpose of describing particular embodiments only, and is not intended to be limiting.

The articles “a” and “an” are used herein to refer to one or more than one (i.e., to at least one) of the grammatical object of the article. By way of example, “a cell” means one cell or more than one cell.

“About” as used herein when referring to a measurable value such as an amount, a temporal duration, and the like, is meant to encompass variations of ±5%, preferably ±1%, and still more preferably +0.1% from the specified value, as such variations are appropriate to perform the disclosed methods.

Embodiments described herein as “comprising” one or more features may also be considered as disclosure of the corresponding embodiments “consisting of” and/or “consisting essentially of” such features, and vice-versa.

Concentrations, amounts, volumes, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range in explicitly recited.

As used herein, the term “prokaryotic host cell” means a prokaryotic cell that is susceptible to transformation, transfection, transduction, or the like, with a nucleic acid construct or expression vector comprising a polynucleotide. The term “prokaryotic host cell” encompasses any progeny that is not identical due to mutations that occur during replication.

As used herein, the term “recombinant cell” or “recombinant host” means a cell or host cell that has been genetically modified or altered to comprise a nucleic acid sequence that is not native to the cell or host cell. In some embodiments the genetic modification comprises integrating the polynucleotide in the genome of the host cell. In further embodiments the polynucleotide is exogenous in the host cell.

As used herein, the term “intermediate” means an intermediate in the production or biosynthesis of a tryptophan or a tryptamine. For example, an intermediate of psilocybin means, e.g., norbaeocystin, baeocystin, 4-hydroxytryptophan, 4-hydroxytryptamine.

As used herein, the term “side product” of a tryptophan or a tryptamine means a side product in the production or biosynthesis of a tryptophan or a tryptamine. For example, a side product of psilocybin means, e.g., aeruginascin, psilocin, norpsilocin, or 4-hydroxy-N,N,N-trimethyltryptamine (4-OH-TMT).

As used herein, the term “derivative” of a compound means a compound with one or more side chain additions or subtractions that still maintains the chemical classification of the compound. For example, a derivative of a tryptophan or a tryptamine means a tryptophan or a tryptamine with one or more side chain additions or subtractions that still maintains the chemical classification of the tryptophan or tryptamine. In some embodiments, the derivative is non-naturally occurring.

The materials, compositions, and methods described herein are intended to be used to provide novel routes for the production of psilocybin and intermediates or side products.

### I. Methods, Vectors, Host Cells and Kits for the Production of a Tryptamine or an Intermediate or a Side Product Thereof

### Methods

Provided is a method for the production of a tryptophan, a tryptamine, or an intermediate, or a side product thereof, or a derivative thereof, comprising contacting a first prokaryotic host cell with one or more expression vectors, wherein each expression vector comprises a gene selected from the group consisting of psiH, CPR, and combinations thereof, contacting a second prokaryotic host cell with one or more expression vectors, wherein each expression vector comprises a gene selected from the group consisting of psiD, a psiK, psiM, and combinations thereof, and co-culturing the first prokaryotic host cell with the second prokaryotic host cell.

In some embodiments, the tryptophan is a non-naturally occurring tryptophan derivative.

In some embodiments, the tryptamine is a non-naturally occurring tryptamine derivative. In further embodiments, the non-naturally occurring tryptamine is a substituted 4-hydroxytryptamine derivative.

In some embodiments, the tryptamine is a psilocybin derivative.

In certain embodiments, the first prokaryotic host cell is selected from the group consisting of Escherichia coli, Corynebacterium glutamicum, Vibrio natriegens, Bacillus subtilis, Bacillus megaterium, Escherichia coli Nissle 1917, Clostridium acetobutlyicum, Streptomyces coelicolor, Lactococcus lactis, Pseudomonas putida, Streptomyces clavuligerus, and Streptomyces venezuelae.

In certain embodiments, the second prokaryotic host cell is selected from the group consisting of Escherichia coli, Corynebacterium glutamicum, Vibrio natriegens, Bacillus subtilis, Bacillus megaterium, Escherichia coli Nissle 1917, Clostridium acetobutlyicum, Streptomyces coelicolor, Lactococcus lactis, Pseudomonas putida, Streptomyces clavuligerus, and Streptomyces venezuelae.

In some embodiments, the first prokaryotic host cell is contacted with the one or more expression vectors, wherein each expression vector comprises a gene selected from the group consisting of psiH, CPR, and combinations thereof, before the second prokaryotic host cell is contacted with the one or more expression vectors, wherein each expression vector comprises a gene selected from the group consisting of psiD, psiK, psiM, and combinations thereof. In some embodiments, the second prokaryotic host cell is contacted with the one or more expression vectors, wherein each expression vector comprises a gene selected from the group consisting of psiD, psiK, psiM, and combinations thereof, before the first prokaryotic host cell is contacted with the one or more expression vectors, wherein each expression vector comprises a gene selected from the group consisting of psiH, CPR, and combinations thereof. In some embodiments, the first prokaryotic host cell is contacted with the one or more expression vectors, wherein each expression vector comprises a gene selected from the group consisting of psiH, CPR, and combinations thereof, at the same time as the second prokaryotic host cell is contacted with the one or more expression vectors, wherein each expression vector comprises a gene selected from the group consisting of psiD, psiK, psiM, and combinations thereof.

In certain embodiments, the psiH gene encodes a polypeptide comprising the amino acid sequence of SEQ ID NO: 10 or a sequence having at least 60%, at least 70%, at least 80%, at least 90%, at least 95%, at least 96%, at least 97%, at least 98%, or at least 99% sequence identity thereto. In certain embodiments, the psiH gene encodes a polypeptide comprising the amino acid sequence of Genbank accession number MF000993 or a sequence having at least 60%, at least 70%, at least 80%, at least 90%, at least 95%, at least 96%, at least 97%, at least 98%, or at least 99% sequence identity thereto. In certain embodiments, the psiH is encoded by a nucleotide sequence comprising SEQ ID NO: 9 or a sequence having at least 60%, at least 70%, at least 80%, at least 90%, at least 95%, at least 96%, at least 97%, at least 98%, or at least 99% sequence identity thereto.

In certain embodiments, the CPR gene encodes a polypeptide comprising the amino acid sequence of SEQ ID NO: 2, 4, 6, 8, or a sequence having at least 60%, at least 70%, at least 80%, at least 90%, at least 95%, at least 96%, at least 97%, at least 98%, or at least 99% sequence identity thereto. In certain embodiments, the CPR gene encodes a polypeptide comprising the amino acid sequence of Genbank accession number AY571340, KAG5165352.1, or a sequence having at least 60%, at least 70%, at least 80%, at least 90%, at least 95%, at least 96%, at least 97%, at least 98%, or at least 99% sequence identity thereto. In certain embodiments, the CPR is encoded by a nucleotide sequence comprising SEQ ID NO: 1, 3, 5, 7, or a sequence having at least 60%, at least 70%, at least 80%, at least 90%, at least 95%, at least 96%, at least 97%, at least 98%, or at least 99% sequence identity thereto.

In certain embodiments, the psiD gene encodes a polypeptide comprising the amino acid sequence of SEQ ID NO: 14 or a sequence having at least 60%, at least 70%, at least 80%, at least 90%, at least 95%, at least 96%, at least 97%, at least 98%, or at least 99% sequence identity thereto. In certain embodiments, the psiD gene encodes a polypeptide comprising the amino acid sequence of Genbank accession number KY984101.1 or a sequence having at least 60%, at least 70%, at least 80%, at least 90%, at least 95%, at least 96%, at least 97%, at least 98%, or at least 99% sequence identity thereto. In certain embodiments, the psiD is encoded by a nucleotide sequence comprising SEQ ID NO: 11 or a sequence having at least 60%, at least 70%, at least 80%, at least 90%, at least 95%, at least 96%, at least 97%, at least 98%, or at least 99% sequence identity thereto.

In certain embodiments, the psiK gene encodes a polypeptide comprising the amino acid sequence of SEQ ID NO: 15 or a sequence having at least 60%, at least 70%, at least 80%, at least 90%, at least 95%, at least 96%, at least 97%, at least 98%, or at least 99% sequence identity thereto. In certain embodiments, the psiK gene encodes a polypeptide comprising the amino acid sequence of Genbank accession number KY984099.1 or a sequence having at least 60%, at least 70%, at least 80%, at least 90%, at least 95%, at least 96%, at least 97%, at least 98%, or at least 99% sequence identity thereto. In certain embodiments, the psiK is encoded by a nucleotide sequence comprising SEQ ID NO: 12 or a sequence having at least 60%, at least 70%, at least 80%, at least 90%, at least 95%, at least 96%, at least 97%, at least 98%, or at least 99% sequence identity thereto.

In certain embodiments, the psiM gene encodes a polypeptide comprising the amino acid sequence of SEQ ID NO: 16 or a sequence having at least 60%, at least 70%, at least 80%, at least 90%, at least 95%, at least 96%, at least 97%, at least 98%, or at least 99% sequence identity thereto. In certain embodiments, the psiM gene encodes a polypeptide comprising the amino acid sequence of Genbank accession number KY984100.1 or a sequence having at least 60%, at least 70%, at least 80%, at least 90%, at least 95%, at least 96%, at least 97%, at least 98%, or at least 99% sequence identity thereto. In certain embodiments, the psiM is encoded by a nucleotide sequence comprising SEQ ID NO: 13 or a sequence having at least 60%, at least 70%, at least 80%, at least 90%, at least 95%, at least 96%, at least 97%, at least 98%, or at least 99% sequence identity thereto.

In certain embodiments, the first prokaryotic cell is contacted with an expression vector comprising a psiH gene, and a CPR gene all under control of a single promoter in operon configuration. In some embodiments, the promoter is selected from the group consisting of G6 mutant T7, H9 mutant T7, H10 mutant T7, C4 mutant T7, consensus T7, Lac, Lac UV5, tac, trc, GAP, and xylA promoter.

In certain embodiments, the first prokaryotic cell is contacted with an expression vector comprising a psiH gene, and a CPR gene, wherein each gene is under control of a separate promoter in pseudooperon or monocistronic configuration. In some embodiments, the promoter is selected from the group consisting of G6 mutant T7, H9 mutant T7, H10 mutant T7, C4 mutant T7, consensus T7, Lac, Lac UV5, tac, trc, GAP, and xylA promoter.

In certain embodiments, the second prokaryotic cell is contacted with an expression vector comprising a psiD gene, a psiK gene, and a psiM gene all under control of a single promoter in operon configuration. In some embodiments, the promoter is selected from the group consisting of G6 mutant T7, H9 mutant T7, H10 mutant T7, C4 mutant T7, consensus T7, Lac, Lac UV5, tac, trc, GAP, and xylA promoter.

In certain embodiments, the second prokaryotic cell is contacted with an expression vector comprising a psiD gene, a psiK gene, and a psiM gene, wherein each gene is under control of a separate promoter in pseudooperon or monocistronic configuration. In some embodiments, each promoter is independently selected from the group consisting of G6 mutant T7, H9 mutant T7, H10 mutant T7, C4 mutant T7, consensus T7, Lac, Lac UV5, tac, trc, GAP, and xylA promoter.

It is envisaged that any intermediate or side product or derivative of a tryptophan or a tryptamine may be produced by any of the methods described herein. In some embodiments, the tryptamine is psilocybin. In some embodiments, the intermediate or side product of psilocybin is norbaeocystin, baeocystin, 4-hydroxytryptophan, 4-hydroxytryptamine, aeruginascin, psilocin, norpsilocin, or 4-hydroxy-N,N,N-trimethyltryptamine (4-OH-TMT).

In some embodiments the intermediate of psilocybin is norbaeocystin, baeocystin, 4-hydroxytryptophan, or 4-hydroxytryptamine. In some embodiments, the side product of psilocybin is aeruginascin, psilocin, norpsilocin, or 4-hydroxy-N,N,N-trimethyltryptamine (4-OH-TMT).

In certain embodiments, the host cell is cultured with a supplement independently selected from the group consisting of serine, tryptamine, tryptophan, indole, and combinations thereof. In certain embodiments, the supplement does not comprise 4-hydroxyindole.

In some embodiments, the indole is

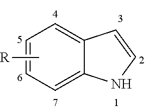

wherein R is independently NH₂, Br, Cl, CN, F, OH, I, OCH₃, CH₃, CF₃, or NO₂.

In certain embodiments, the supplement does not comprise an amino acid. In certain exemplary embodiments, the supplement is fed continuously to the host cell. In further embodiments, the host cell is grown in an actively growing culture. Continuous feeding is accomplished by using a series of syringe and/or peristaltic pumps whose outlet flow is directly connected to the bioreactor. The set point of these supplement addition pumps is adjusted in response to real-time measurement of cell biomass and specific metabolic levels using UV-vis absorption and HPLC analysis, respectively. The fed-batch fermentation process is focused on maximizing production of target metabolites through harnessing the ability of an actively growing and replicating cell culture to regenerate key co-factors and precursors which are critical to the biosynthesis of target metabolites. This process notably does not involve the centrifugal concentration and reconstitution of cell biomass to artificially higher cell density and/or into production media that was not used to build the initial biomass. The production process involves the inoculation of the reactor from an overnight preculture at low optical density, followed by exponential phase growth entering into a fed-batch phase of production, culminating in a high cell density culture.

The tryptophan, tryptamine and intermediate or side products or derivatives are found extracellularly in the fermentation broth. In certain embodiments, the tryptophan, tryptamine and intermediate or side products or derivatives are isolated. These target products can be collected through drying the fermentation broth after centrifugation to remove the cell biomass. The resulting dry product can be extracted to further purify the target compounds. Alternatively, the products can be extracted from the liquid cell culture broth using a solvent which is immiscible with water and partitions psilocybin or any of the intermediate or side products into the organic phase. Furthermore, contaminants from the fermentation broth can be removed through extraction leaving the psilocybin and/or intermediate or side products in the aqueous phase for collection after drying or crystallization procedures.

In certain embodiments, the methods described herein result in a titer of tryptophan derivatives, tryptamine derivatives and/or psilocybin derivatives of about 0.5 to about 500 mg/L. In some embodiments, the methods described herein result in a titer of tryptophan derivatives, tryptamine derivatives and/or psilocybin derivatives of about 0.5 to about 300 mg/L. In yet further embodiments, the methods described herein result in a titer of tryptophan derivatives, tryptamine derivatives and/or psilocybin derivatives of about 0.5 to about 200 mg/L. In certain embodiments, the methods described herein result in a titer of tryptophan derivatives, tryptamine derivatives and/or psilocybin derivatives of about 30 to about 200 mg/L. In further embodiments, the methods described herein result in a titer of tryptophan derivatives, tryptamine derivatives and/or psilocybin derivatives of about 100 mg/L.

In certain embodiments, the methods described herein result in a molar yield of tryptophan derivatives, tryptamine derivatives and/or psilocybin derivatives of about 10% to about 100%. In some embodiments, the methods described herein result in a molar yield of tryptophan derivatives, tryptamine derivatives and/or psilocybin derivatives of about 20% to about 80%. In yet further embodiments, the methods described herein result in a molar yield of tryptophan derivatives, tryptamine derivatives and/or psilocybin derivatives of about 30% to about 70%. In certain embodiments, the methods described herein result in a molar yield of tryptophan derivatives, tryptamine derivatives and/or psilocybin derivatives of about 40% to about 60%. In further embodiments, the methods described herein result in a molar yield of tryptophan derivatives, tryptamine derivatives and/or psilocybin derivatives of about 50%.

Also provided is a method for the production of a tryptophan, a tryptamine, or an intermediate or a side product thereof, or a derivative thereof, comprising: contacting a prokaryotic host cell with one or more expression vectors, wherein each expression vector comprises a gene selected from the group consisting of psiH, CPR, psiD, psiK, psiM, and combinations thereof.

In some embodiments, the tryptophan, tryptamine, intermediate or side product is a non-naturally occurring derivative.

In some embodiments, the tryptamine is a non-naturally occurring tryptamine derivative. In further embodiments, the non-naturally occurring tryptamine derivative is a substituted 4-hydroxytryptamine.

In some embodiments, the tryptamine is a psilocybin derivative.

In certain embodiments, the psiH gene encodes a polypeptide comprising the amino acid sequence of SEQ ID NO: 10 or a sequence having at least 60%, at least 70%, at least 80%, at least 90%, at least 95%, at least 96%, at least 97%, at least 98%, or at least 99% sequence identity thereto. In certain embodiments, the psiH gene encodes a polypeptide comprising the amino acid sequence of Genbank accession number MF000993 or a sequence having at least 60%, at least 70%, at least 80%, at least 90%, at least 95%, at least 96%, at least 97%, at least 98%, or at least 99% sequence identity thereto. In certain embodiments, the psiH is encoded by a nucleotide sequence comprising SEQ ID NO: 9 or a sequence having at least 60%, at least 70%, at least 80%, at least 90%, at least 95%, at least 96%, at least 97%, at least 98%, or at least 99% sequence identity thereto.

In certain embodiments, the CPR gene encodes a polypeptide comprising the amino acid sequence of SEQ ID NO: 2, 4, 6, 8, or a sequence having at least 60%, at least 70%, at least 80%, at least 90%, at least 95%, at least 96%, at least 97%, at least 98%, or at least 99% sequence identity thereto. In certain embodiments, the CPR gene encodes a polypeptide comprising the amino acid sequence of Genbank accession number AY571340, KAG5165352.1, or a sequence having at least 60%, at least 70%, at least 80%, at least 90%, at least 95%, at least 96%, at least 97%, at least 98%, or at least 99% sequence identity thereto. In certain embodiments, the CPR is encoded by a nucleotide sequence comprising SEQ ID NO: 1, 3, 5, 7, or a sequence having at least 60%, at least 70%, at least 80%, at least 90%, at least 95%, at least 96%, at least 97%, at least 98%, or at least 99% sequence identity thereto.

In certain embodiments, the psiD gene encodes a polypeptide comprising the amino acid sequence of SEQ ID NO: 14 or a sequence having at least 60%, at least 70%, at least 80%, at least 90%, at least 95%, at least 96%, at least 97%, at least 98%, or at least 99% sequence identity thereto. In certain embodiments, the psiD gene encodes a polypeptide comprising the amino acid sequence of Genbank accession number KY984101.1 or a sequence having at least 60%, at least 70%, at least 80%, at least 90%, at least 95%, at least 96%, at least 97%, at least 98%, or at least 99% sequence identity thereto. In certain embodiments, the psiD is encoded by a nucleotide sequence comprising SEQ ID NO: 11 or a sequence having at least 60%, at least 70%, at least 80%, at least 90%, at least 95%, at least 96%, at least 97%, at least 98%, or at least 99% sequence identity thereto.

In certain embodiments, the psiK gene encodes a polypeptide comprising the amino acid sequence of SEQ ID NO: 15 or a sequence having at least 60%, at least 70%, at least 80%, at least 90%, at least 95%, at least 96%, at least 97%, at least 98%, or at least 99% sequence identity thereto. In certain embodiments, the psiK gene encodes a polypeptide comprising the amino acid sequence of Genbank accession number KY984099.1 or a sequence having at least 60%, at least 70%, at least 80%, at least 90%, at least 95%, at least 96%, at least 97%, at least 98%, or at least 99% sequence identity thereto. In certain embodiments, the psiK is encoded by a nucleotide sequence comprising SEQ ID NO: 12 or a sequence having at least 60%, at least 70%, at least 80%, at least 90%, at least 95%, at least 96%, at least 97%, at least 98%, or at least 99% sequence identity thereto.

In certain embodiments, the psiM gene encodes a polypeptide comprising the amino acid sequence of SEQ ID NO: 16 or a sequence having at least 60%, at least 70%, at least 80%, at least 90%, at least 95%, at least 96%, at least 97%, at least 98%, or at least 99% sequence identity thereto. In certain embodiments, the psiM gene encodes a polypeptide comprising the amino acid sequence of Genbank accession number KY984100.1 or a sequence having at least 60%, at least 70%, at least 80%, at least 90%, at least 95%, at least 96%, at least 97%, at least 98%, or at least 99% sequence identity thereto. In certain embodiments, the psiM is encoded by a nucleotide sequence comprising SEQ ID NO: 13 or a sequence having at least 60%, at least 70%, at least 80%, at least 90%, at least 95%, at least 96%, at least 97%, at least 98%, or at least 99% sequence identity thereto.

In certain embodiments, the prokaryotic cell is contacted with an expression vector comprising a psiH gene, a CPR gene, a psiD gene, a psiK gene, and a psiM gene, all under control of a single promoter in operon configuration. In some embodiments, the promoter is selected from the group consisting of G6 mutant T7, H9 mutant T7, H10 mutant T7, C4 mutant T7, consensus T7, Lac, Lac UV5, tac, trc, GAP, and xylA promoter.

In certain embodiments, the prokaryotic cell is contacted with an expression vector comprising a psiH gene, a CPR gene, a psiD gene, a psiK gene, and a psiM gene, wherein each gene is under control of a separate promoter in pseudooperon or monocistronic configuration. In some embodiments, the promoter is selected from the group consisting of G6 mutant T7, H9 mutant T7, H10 mutant T7, C4 mutant T7, consensus T7, Lac, Lac UV5, tac, trc, GAP, and xylA promoter.

In some embodiments, the prokaryotic cell is selected from the group consisting of Escherichia coli, Corynebacterium glutamicum, Vibrio natriegens, Bacillus subtilis, Bacillus megaterium, Escherichia coli Nissle 1917, Clostridium acetobutlyicum, Streptomyces coelicolor, Lactococcus lactis, Pseudomonas putida, Streptomyces clavuligerus, and Streptomyces venezuelae.

Also provided is a method for the production of a tryptophan, a tryptamine, or an intermediate or a side product thereof, or a derivative thereof, comprising: contacting a prokaryotic host cell with one or more expression vectors, wherein each expression vector comprises a gene selected from the group consisting of psiD, psiH, CPR, and combinations thereof, and culturing the host cell.

In some embodiments, the tryptophan, tryptamine, intermediate or side product is a non-naturally occurring derivative.

In some embodiments, the tryptamine is a non-naturally occurring tryptamine derivative. In further embodiments, the non-naturally occurring tryptamine derivative is a substituted 4-hydroxytryptamine.

In some embodiments, the tryptamine is 4-hydroxytryptophan.

In certain embodiments, the psiD gene encodes a polypeptide comprising the amino acid sequence of SEQ ID NO: 14 or a sequence having at least 60%, at least 70%, at least 80%, at least 90%, at least 95%, at least 96%, at least 97%, at least 98%, or at least 99% sequence identity thereto. In certain embodiments, the psiD gene encodes a polypeptide comprising the amino acid sequence of Genbank accession number KY984101.1 or a sequence having at least 60%, at least 70%, at least 80%, at least 90%, at least 95%, at least 96%, at least 97%, at least 98%, or at least 99% sequence identity thereto. In certain embodiments, the psiD is encoded by a nucleotide sequence comprising SEQ ID NO: 11 or a sequence having at least 60%, at least 70%, at least 80%, at least 90%, at least 95%, at least 96%, at least 97%, at least 98%, or at least 99% sequence identity thereto.

In certain embodiments, the psiH gene encodes a polypeptide comprising the amino acid sequence of SEQ ID NO: 10 or a sequence having at least 60%, at least 70%, at least 80%, at least 90%, at least 95%, at least 96%, at least 97%, at least 98%, or at least 99% sequence identity thereto. In certain embodiments, the psiH gene encodes a polypeptide comprising the amino acid sequence of Genbank accession number MF000993 or a sequence having at least 60%, at least 70%, at least 80%, at least 90%, at least 95%, at least 96%, at least 97%, at least 98%, or at least 99% sequence identity thereto. In certain embodiments, the psiH is encoded by a nucleotide sequence comprising SEQ ID NO: 9 or a sequence having at least 60%, at least 70%, at least 80%, at least 90%, at least 95%, at least 96%, at least 97%, at least 98%, or at least 99% sequence identity thereto.

In certain embodiments, the CPR gene encodes a polypeptide comprising the amino acid sequence of SEQ ID NO: 2, 4, 6, 8, or a sequence having at least 60%, at least 70%, at least 80%, at least 90%, at least 95%, at least 96%, at least 97%, at least 98%, or at least 99% sequence identity thereto. In certain embodiments, the CPR gene encodes a polypeptide comprising the amino acid sequence of Genbank accession number AY571340, KAG5165352.1, or a sequence having at least 60%, at least 70%, at least 80%, at least 90%, at least 95%, at least 96%, at least 97%, at least 98%, or at least 99% sequence identity thereto. In certain embodiments, the CPR is encoded by a nucleotide sequence comprising SEQ ID NO: 1, 3, 5, 7, or a sequence having at least 60%, at least 70%, at least 80%, at least 90%, at least 95%, at least 96%, at least 97%, at least 98%, or at least 99% sequence identity thereto.

In certain embodiments, the prokaryotic cell is contacted with an expression vector comprising a psiD gene, a psiH gene, and a CPR gene all under control of a single promoter in operon configuration. In some embodiments, the promoter is selected from the group consisting of G6 mutant T7, H9 mutant T7, H10 mutant T7, C4 mutant T7, consensus T7, Lac, Lac UV5, tac, trc, GAP, and xylA promoter.

In certain embodiments, the prokaryotic cell is contacted with an expression vector comprising a psiD gene, a psiH gene, and a CPR gene, wherein each gene is under control of a separate promoter in pseudooperon or monocistronic configuration. In some embodiments, the promoter is selected from the group consisting of G6 mutant T7, H9 mutant T7, H10 mutant T7, C4 mutant T7, consensus T7, Lac, Lac UV5, tac, trc, GAP, and xylA promoter.

In some embodiments, the prokaryotic cell is selected from the group consisting of Escherichia coli, Corynebacterium glutamicum, Vibrio natriegens, Bacillus subtilis, Bacillus megaterium, Escherichia coli Nissle 1917, Clostridium acetobutlyicum, Streptomyces coelicolor, Lactococcus lactis, Pseudomonas putida, Streptomyces clavuligerus, and Streptomyces venezuelae.

It is envisaged that any intermediate or side product of a tryptophan or a tryptamine may be produced by any of the methods described herein. In some embodiments, the tryptamine is psilocybin. In some embodiments, the intermediate or side product of psilocybin is norbaeocystin, baeocystin, 4-hydroxytryptophan, 4-hydroxytryptamine, aeruginascin, psilocin, norpsilocin, or 4-hydroxy-N,N,N-trimethyltryptamine (4-OH-TMT). In some embodiments the intermediate of psilocybin is norbaeocystin, baeocystin, 4-hydroxytryptophan, or 4-hydroxytryptamine. In some embodiments, the side product of psilocybin is aeruginascin, psilocin, norpsilocin, or 4-hydroxy-N,N,N-trimethyltryptamine (4-OH-TMT).

It is also envisaged that any non-naturally occurring tryoptophan, tryptamine, intermediate or side product may be produced by any of the methods described herein. In some embodiments, the non-naturally occurring tryptamine is 6-bromopsilocybin, 7-bromopsilocybin, 6-chloropsilocybin, 7-chloropsilocybin, 5-fluoropsilocybin, 6-fluoropsilocybin, 7-fluoropsilocybin, 6-iodopsilocybin, 7-iodopsilocybin, 6-methoxypsilocybin, 7-methoxypsilocybin, 6-methylpsilocybin, or 7-methylpsilocybin.

In some embodiments, the culture is supplemented with a supplement independently selected from the group consisting of serine, tryptamine, tryptophan, indole, combinations thereof, and derivatives thereof.

### Recombinant Prokaryotic Cells for the Production of a Tryptophan, a Tryptamine or an Intermediate or a Side Product Thereof

Provided is a recombinant prokaryotic cell comprising one or more expression vectors, wherein each expression vector comprises a gene selected from the group consisting of psiH, CPR, and combinations thereof.

In certain embodiments, the recombinant prokaryotic cell is selected from the group consisting of Escherichia coli, Corynebacterium glutamicum, Vibrio natriegens, Bacillus subtilis, Bacillus megaterium, Escherichia coli Nissle 1917, Clostridium acetobutlyicum, Streptomyces coelicolor, Lactococcus lactis, Pseudomonas putida, Streptomyces clavuligerus, and Streptomyces venezuelae.

In certain embodiments, the psiH gene encodes a polypeptide comprising the amino acid sequence of SEQ ID NO: 10 or a sequence having at least 60%, at least 70%, at least 80%, at least 90%, at least 95%, at least 96%, at least 97%, at least 98%, or at least 99% sequence identity thereto. In certain embodiments, the psiH gene encodes a polypeptide comprising the amino acid sequence of Genbank accession number MF000993 or a sequence having at least 60%, at least 70%, at least 80%, at least 90%, at least 95%, at least 96%, at least 97%, at least 98%, or at least 99% sequence identity thereto. In certain embodiments, the psiH is encoded by a nucleotide sequence comprising SEQ ID NO: 9 or a sequence having at least 60%, at least 70%, at least 80%, at least 90%, at least 95%, at least 96%, at least 97%, at least 98%, or at least 99% sequence identity thereto.

In certain embodiments, the CPR gene encodes a polypeptide comprising the amino acid sequence of SEQ ID NO: 2, 4, 6, 8, or a sequence having at least 60%, at least 70%, at least 80%, at least 90%, at least 95%, at least 96%, at least 97%, at least 98%, or at least 99% sequence identity thereto. In certain embodiments, the CPR gene encodes a polypeptide comprising the amino acid sequence of Genbank accession number AY571340, KAG5165352.1, or a sequence having at least 60%, at least 70%, at least 80%, at least 90%, at least 95%, at least 96%, at least 97%, at least 98%, or at least 99% sequence identity thereto. In certain embodiments, the CPR is encoded by a nucleotide sequence comprising SEQ ID NO: 1, 3, 5, 7, or a sequence having at least 60%, at least 70%, at least 80%, at least 90%, at least 95%, at least 96%, at least 97%, at least 98%, or at least 99% sequence identity thereto.

In certain embodiments, the prokaryotic cell is contacted with an expression vector comprising a psiH gene, and a CPR gene all under control of a single promoter in operon configuration. In certain embodiments, the prokaryotic cell is contacted with an expression vector comprising a psiH gene, and a CPR gene, wherein each gene is under control of a separate promoter in pseudooperon configuration. In certain embodiments, each gene is in monocistronic configuration, wherein each gene has a promoter and a terminator. Any configuration or arrangement of promoters and terminators is envisaged.

In some embodiments, the promoter is selected from the group consisting of G6 mutant T7, H9 mutant T7, H10 mutant T7, C4 mutant T7, consensus T7, Lac, Lac UV5, tac, trc, GAP, and xylA promoter.

### Expression Vectors

Provided is a vector for introducing at least one gene; the gene may be selected from: psiH, CPR, and combinations thereof.

In certain embodiments, the psiH gene encodes a polypeptide comprising the amino acid sequence of SEQ ID NO: 10 or a sequence having at least 60%, at least 70%, at least 80%, at least 90%, at least 95%, at least 96%, at least 97%, at least 98%, or at least 99% sequence identity thereto. In certain embodiments, the psiH gene encodes a polypeptide comprising the amino acid sequence of Genbank accession number MF000993 or a sequence having at least 60%, at least 70%, at least 80%, at least 90%, at least 95%, at least 96%, at least 97%, at least 98%, or at least 99% sequence identity thereto. In certain embodiments, the psiH is encoded by a nucleotide sequence comprising SEQ ID NO: 9 or a sequence having at least 60%, at least 70%, at least 80%, at least 90%, at least 95%, at least 96%, at least 97%, at least 98%, or at least 99% sequence identity thereto.

In certain embodiments, the CPR gene encodes a polypeptide comprising the amino acid sequence of SEQ ID NO: 2, 4, 6, 8, or a sequence having at least 60%, at least 70%, at least 80%, at least 90%, at least 95%, at least 96%, at least 97%, at least 98%, or at least 99% sequence identity thereto. In certain embodiments, the CPR gene encodes a polypeptide comprising the amino acid sequence of Genbank accession number AY571340, KAG5165352.1, or a sequence having at least 60%, at least 70%, at least 80%, at least 90%, at least 95%, at least 96%, at least 97%, at least 98%, or at least 99% sequence identity thereto. In certain embodiments, the CPR is encoded by a nucleotide sequence comprising SEQ ID NO: 1, 3, 5, 7, or a sequence having at least 60%, at least 70%, at least 80%, at least 90%, at least 95%, at least 96%, at least 97%, at least 98%, or at least 99% sequence identity thereto.

In certain embodiments, the expression vector comprises a psiH gene, and a CPR gene all under control of a single promoter in operon configuration. In certain embodiments, the expression vector comprises a psiH gene, and a CPR gene, wherein each gene is under control of a separate promoter in pseudooperon configuration. In certain embodiments, each gene is in monocistronic configuration, wherein each gene has a promoter and a terminator. Any configuration or arrangement of promoters and terminators is envisaged.

In some embodiments, the promoter is selected from the group consisting of G6 mutant T7, H9 mutant T7, H10 mutant T7, C4 mutant T7, consensus T7, Lac, Lac UV5, tac, trc, GAP, and xylA promoter.

### Kits

Provided is a transfection kit comprising an expression vector as described herein. Such a kit may comprise a carrying means being compartmentalized to receive in close confinement one or more container means such as, e.g., vials or test tubes. Each of such container means comprises components or a mixture of components needed to perform a transfection. Such kits may include, for example, one or more components selected from vectors, cells, reagents, lipid-aggregate forming compounds, transfection enhancers, or biologically active molecules.

### Examples

### Materials and Methods

### Bacterial Strains, Vectors, and Media

E col; DH5a was used for plasmid propagation and cloning, while BL21 Star™ (DE3) was used as the host for all chemical production experiments. Luria Broth (LB) was used as the base media during cloning. Andrew's Magic Media (AMM) (He et al., 2015) with or without 3-(Morpholin-4-yl)propane-1-sulfonic acid (MOPS) was used for all production experimnents, including preculture growth from freezer stocks or LB-agar plates. Plasmid transformations were carried out using either standard chemical- or electro-competent cell preparation protocols. Ampicillin (80 μg/mL), streptomycin (50 μg/mL), or chloramphenicol (25 μg/mL) was added to the culture media when appropriate for plasmid retention. All plasmids, strains, and accompanying descriptions can be found in Table 1.

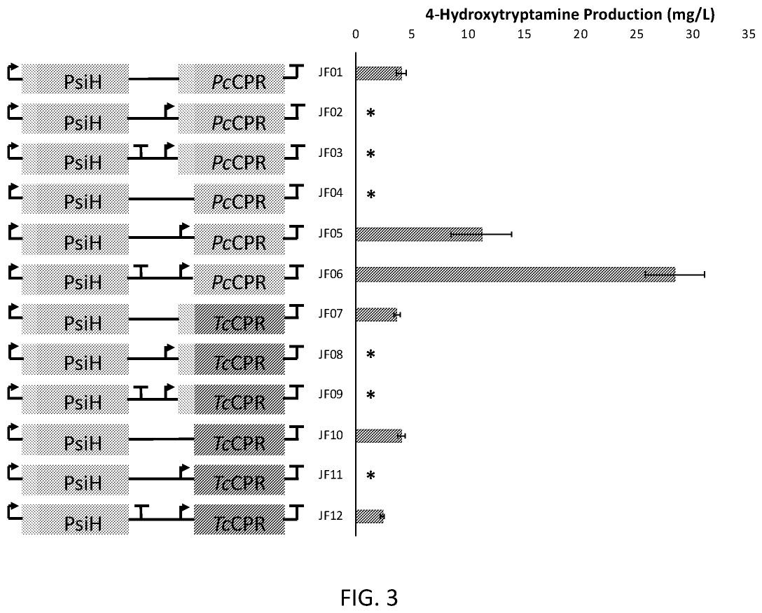

Cytochrome P450 reductase (CPR) variants were synthesized as linear double stranded DNA ordered from Genewiz (now Azenta). Psilocvbe cubensis and Taxus cuspidata CPRs with and without N-terminal solubility tags were PCR amplified with primers P1-P3, P9-P10 (Table 3). The PCR fragments and the empty pETM6-SDM2x vector were then digested with NdeI and XhoI gel extracted, and ligated to form pETM6-SDM2x-PcCPR and pETM6-SDM2x-TcCPR, each with and without the solubility tag. Plasmid DNA containing the coding sequence for PsiH was obtained as a gift from Dirk Hoffmeister (Hoefgen et al., 2018) on plasmid pJF25. Site directed mutagenesis was performed on this plasmid to remove an internal NdeI recognition sequence using primers P13 and P14. The resulting gene was then PCR amplified using primers P11 and P12 (Table 3). The PCR fragment and empty pETM6-SDM2x vector were then digested with NdeI and XhoI, gel extracted, and ligated to form pETM6-SDM2x-PsiH. All single gene vectors were sequence verified using Sanger sequencing using primers P4-P8 (Table 3) as needed (Genewiz, now Azenta). The twelve pathway modules (FIG. 3) were assembled using a modified ePathBrick isocaudomer-based system as previously described (Adams et al., 2019; Xu et al., 2012), resulting in plasmids 32-43, Table 1.

The PsiD transcriptional library comprising nine vector and promoter combinations was constructed similarly to that described above. These single gene constructs were assembled via traditional restriction ligation cloning using NdeI and XhoI. pETM6 plasmid backbones were constructed containing four T7 mutant promoters G6, H9, H10, and C4, (Jones et al., 2015) along with consensus T7 (Xu et al., 2012) and ampicillin resistance. pACM4 and pCDM4 vectors contain the consensus T7 promoter and chloramphenicol and streptomycin resistance (Xu et al., 2012), respectively. pXylA and pXPA vectors contain constitutive promoters, XylA and GAP, and an ampicillin resistance cassette (Englaender et al., 2017; Xu et al., 2014).

### Screening Conditions for Hydroxylase Module Development

Screening was performed in 2 mL working volume cultures in 48-well plates, started at 37° C. upon inoculation. The culture was then induced with 1 mM isopropyl β-D-1-thiogalactopyranoside (IPTG) and transferred to 30° C. after 6 hours of growth. AMM, without casamino acids, and ampicillin were used for preliminary and co-culture experiments. Supplements of tryptanine (40 mg/L), tryptophan (40 mg/L), or indole (40 mg/L) were added as noted. Overnight precultures cultures were grown using an agar plate or freezer stock culture in AMM with ampicillin for 12-14 hours in a shaking incubator at 37° C. Samples were subjected to HPLC and MS analysis after 24 hours and/or 48 hours post inoculation as noted.

### Screening Conditions for TrpB and PsiD Substrate Specificity

Four previously constructed BL21 Star™ (DE3) cell lines with pETM6-SDM2x plasmid backbones were used, each with endogenous Escherichia coli (E. coli) tryptophan synthase beta subunit (TrpB); (1) no gene insert (empty vector), (2) PsiD gene insert with T7 promoter, (3) PsiD-PsiK gene insert with C4 promoter in operon configuration (pNor, Norbaeocystin Production Strain) (Adams et al., 2022), and (4) PsiD-PsiK-PsiM gene insert with H10 promoter in operon configuration (pSilo16, Psilocybin Production Strain; See WO 2021/086513) (Adams et al., 2019). For the PsiD transcriptional optimization study, each of the nine previously described library members were used, in lieu of the strains above. Overnight cultures were grown in Andrew's Magic Media (AMM) without MOPS, appropriate antibiotics for plasmid retention, and were inoculated from freezer stocks. Experimental cultures were incubated in covered 48-well plates at 37° C. in a shaking incubator. AMM media consistent with above was inoculated at 2% final volume of respective overnight culture and 2 mL aliquoted to each well. Indole derivatives were added to each well, except negative control, at 100 mg/L using 5 uL from a 40 mg/mL stock in EtOH. 4-Nitroindole at 13.3 mg/mL in EtOH was added using 15 uL due to solubility limitations. Cultures were induced at 2 hours using 1 mM ITPG. Cultures were sampled at 24 hours and subjected to LC-MS analysis as described below. Cultures with serine added were identical to those without, except for adding 4.5 g/L of serine prior to inoculation.

Some substituted indoles caused significant culture growth retardation, when compared to others. The growth deficiency is likely a result of toxicity effects of the indole derivatives, rather than tryptophan derivatives, as strains that produce little or no tryptophan products still exhibit reduced cell growth. The majority of indoles that exhibit this phenotype are halogenated, including; 4-aminoindole, 4-bromoindole, 5-bromoindole, 6-bromoindole, 4-chloroindole, 5-chloroindole, 6-chloroindole, 4-fluoroindole, 5-fluoroindole, 6-fluoroindole, 7-fluoroindole, 4-iodoindole, 5-iodoindole, 6-iodoindole, 4-methylindole, and 6-nitroindole. These indoles were added six hours after inoculation at 50 mg/L final concentration and resulted in growth more in line with other supplements, along with higher indole processing. Other indoles may have slowed cell growth, but the differences were not easily distinguishable visually as OD600 reading were not taken for every sample.

### Co-Cultures

Co-culture experiments primarily utilized the previously reported Psilocybin Production Strain (pSilo16) and the BL21 Star™ (DE3) pETM6-SDM2X-PsiH-PcCPR strain (JF06 or PsiH strain) to identify psilocybin variants. Co-culture experiments were also performed that utilized the Norbaeocystin Production Strain (pNor) and the PsiH strain to identify norbaeocystin variants, along with a higher performing PsiD strain and the PsiH strain to identify 4-hydroxytryptamine variants. Co-cultures were grown under the same conditions, except the following. Overnight cultures were incubated at 37° C., while experimental cultures were incubated in flasks at 30° C. with addition of 4 g/L of serine and methionine. The 2% volume of overnight culture added was divided between the psilocybin production strain and the PsiH strain at 15% and 85% respectively, unless otherwise noted. Samples were taken at 24, 48, and 72 hours, and analyzed on LC-MS as described below.

### Analytical Methods

Metabolite analysis was performed on a Thermo Scientific Ultimate 3000 High-Performance Liquid Chromatography (HPLC) system equipped with Diode Array Detector (DAD) and Thermo Scientific ISQ™ EC single quadrupole mass spectrometer (MS). Samples were prepared for HPLC and LC-MS analysis by centrifugation at 15,000×g for 5 min. A volume of 2 μL of the resulting supernatant was then injected for LC-MS analysis. Substituted indole standards were created by addition of substituted indoles to AMM at concentrations of 100 mg/L.

Quantification of aromatic metabolites was performed using absorbance at 280 nm from the DAD and the metabolites were separated using an Agilent Zorbax Eclipse XDB-C18 analytical column (3.0 mm×250 mm, 5 μm) with mobile phases of water (A) and acetonitrile (B) both containing 0.1% formic acid at a rate of 1 mL/min: 0 min, 5% B; 0.43 min, 5% B; 5.15 min, 19% B; 6.44 min, 100% B; 7.73 min, 100% B; 7.73 min, 5% B; 9.87 min, 5% B. This method resulted in the following observed retention times as verified by analytical standards (when commercially available) and MS analysis (as described below): 4-hydroxyindole (6.6 min), 4-hydroxytryptophan (3.4 min), 4-hydroxytryptamine (3.2 min), norbaeocystin (1.6 min), baeocystin (1.9 min) and psilocybin (2.2 min). Some samples with tryptophan and tryptamine products that eluted very early were also analyzed using an Agilent Polaris C18-A analytical column (4.6 mm×250 mm, 5 μm) with mobile phases of water (A) and acetonitrile (B) both containing 0.1% formic acid at a rate of 1 mL/min: 0 min, 5% B; 0.43 min, 5% B; 5.15 min, 40% B; 6.44 min, 100% B; 7.73 min, 100% B; 7.73 min, 5% B; 9.87 min, 5% B. These conditions provided better retention and separation of polar compounds and were used for aminoindole product quantification in TrpB and PsiD substrate flexibility studies.

Liquid Chromatography Mass Spectrometry (LC-MS) data was collected where the full MS scan was used to provide an extracted ion chromatogram (EIC) of our compounds of interest. Analytes were measured in positive ion mode at the flow rate, solvent gradient, and column conditions described above. The instrument was equipped with a heated electrospray ionization (HESI) source and supplied ≥99% purity nitrogen from a Peak Scientific Genius XE 35 laboratory nitrogen generator. The source and detector conditions were as follows: sheath gas pressure of 80.0 psig, auxiliary gas pressure of 9.7 psig, sweep gas pressure of 0.5 psig, foreline vacuum pump pressure of 1.55 Torr, vaporizer temperature of 500° C., ion transfer tube temperature of 300° C., source voltage of 3049 V, and source current of 15.90 μA.

### Quantification of TrpB and PsiD Substrate Flexibility

Without commercially available standards for many of these compounds absolute quantification of enzymatic products is not possible. Peak areas from one substituted indole could not be compared directly to peak areas from another substituted indole due to significant differences in extinction coefficients. Relative quantification was performed by comparing product UV 280 nm peak areas from HPLC analysis (mAU*min) to that of the corresponding substituted indole. TrpB proficiencies with and without serine were calculated by dividing the tryptophan product peak areas by the total peak areas from the indole and tryptophan peaks. Tryptophan and indole peak areas were determined from duplicate samples with indole and subtraction of any time-correlated peak areas found in the samples without indole addition. Any other indole related peak areas found in indole samples were included in the denominator. Indole related peaks were defined as peaks found in indole samples, but absent from samples without indole addition. Similar calculations were performed for PsiD proficiencies where tryptamine peak areas were divided by the total area of associated tryptamine and tryptophan peaks. Indole utilization was also calculated for PsiD proficiencies where the remaining indole peak areas were divided by the initial peak areas.

### Example 1: Evaluation of Transcriptionally and Translationally Varied PsiH-Containing Constructs

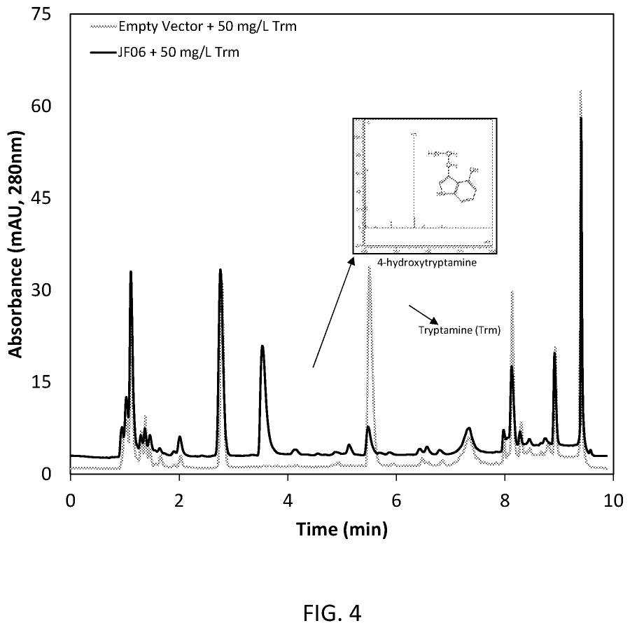

Twelve pathway modules were constructed containing PsiH from Psilocybe cubensis and either CPR from Psilocybe cubensis or Taxus cuspidate. These two variants were chosen due to Psilocybe cubensis being the native reductase partner in the mushroom host, while the Taxus cuspidate variant has been previously shown to enable high P450 activity in E. coli (Biggs et al., 2016; Fricke et al., 2017). In addition to gene source variation, the CPR was also cloned with and without an N-term solubility tag in operon, pseudooperon, and monocistronic configurations. All twelve pathway variants showed measurable activity above the limit of detection by mass spectrometry. One construct, JF06, demonstrated the highest 4-hydroxytryptamine production of 28.4-2.5 mg/L when provided a tryptamine supplement (FIG. 3). The JF06 construct was a PsiH-PcCPR construct in monocistronic configuration with the CPR not containing the solubility tag. This strain was subjected to further LCMS screening to confirm the presence of presence of 4-hydroxytryptamrine (FIG. 4). Here, JF06 (top trace) is shown to produce 4-hydroxytryptamine (rt 3.6 min, m/z 177), while the empty vector control culture produces no 4-hydroxytryptamine (bottom trace). The JF06 construct was taken forward for further screening.

### Example 2: Screening of Fermentation Condition for JF06

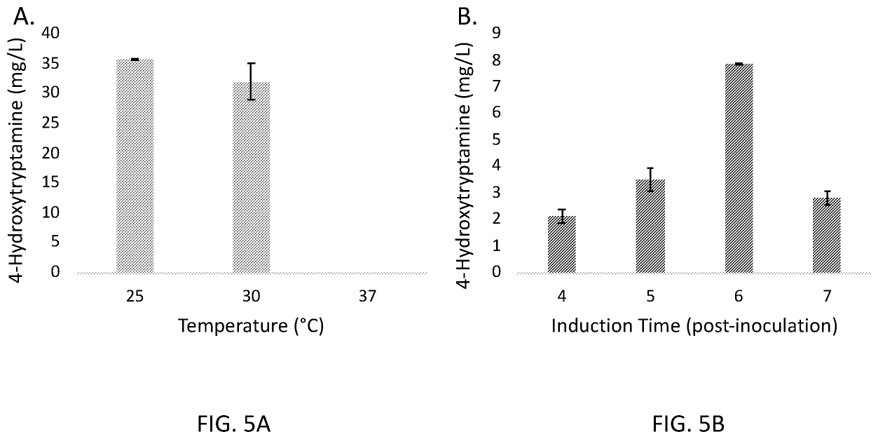

Fermentation temperature and induction timing were varied for the JF06 construct to determine the conditions for the optimal module performance. Temperature of 25, 30, and 37° C. (FIG. 5A) and induction points of 4, 5, 6, and 7 hours post inoculation (FIG. 5B) were screened. These results indicated optimal performance at 25° C. and 6 hours, respectively.

This information was used to inform the development of de novo psilocybin production pathways. It is important to note that previous studies have identified that 37° C. and 4 hours was optimal for psilocybin production from 4-hydroxyindole. Further optimization will be required to balance tradeoffs between the two modules to enable the best de novo psilocybin production conditions.

### Example 3: Evaluation of Co-Culture Proof-of-Principle Approach

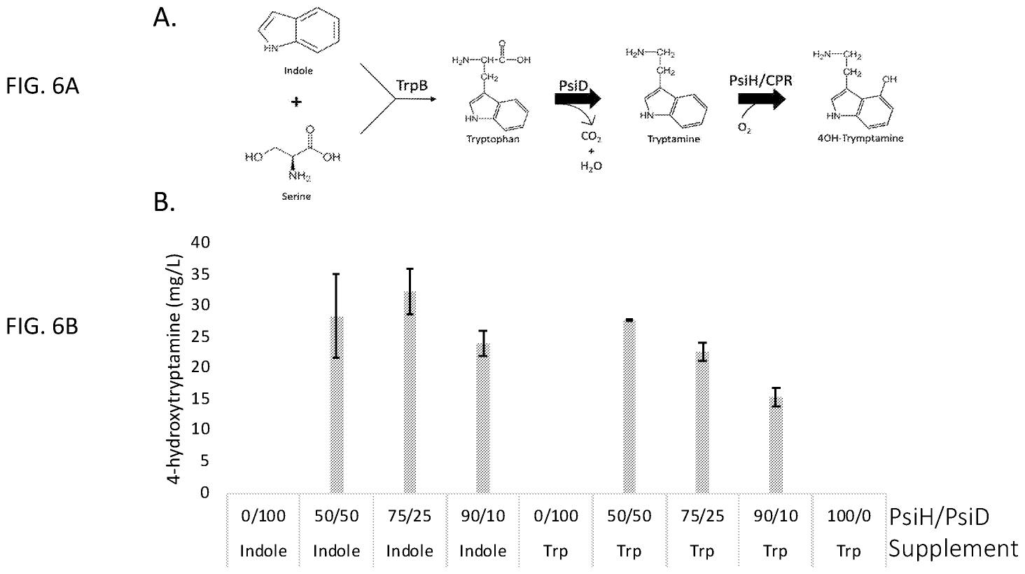

A basic co-culture approach leveraging the previously described F06 strain and an E. coli strain expressing the decarboxylase, psiD, was developed to determine the feasibility of using a co-culture platform for future proof-of-principle studies. In this screening approach, three co-culture inoculation ratios were selected (JF06:PsiD): 50:50, 75:25, and 90:10. These ratios were selected to have a larger concentration of the JF06 module due to previous studies indicating that in monoculture the activity of the PsiD module was much greater than that of the JF06 module (Adams et al., 2019). Additionally, two different supplements (Indole and Tryptophan) were tested individually to boost overall production from the joint pathway. Interestingly, both supplement conditions resulted in similar production of 4-hydroxytryptamine and the inoculation ratios trended towards higher production with inoculation ratios with more PsiD content (FIG. 6B). This study demonstrated the feasibility of the co-culture screening approach and set the groundwork for the full de novo psilocybin pathway to be implemented.

### Example 4: De Novo Psilocybin Production in K Coli

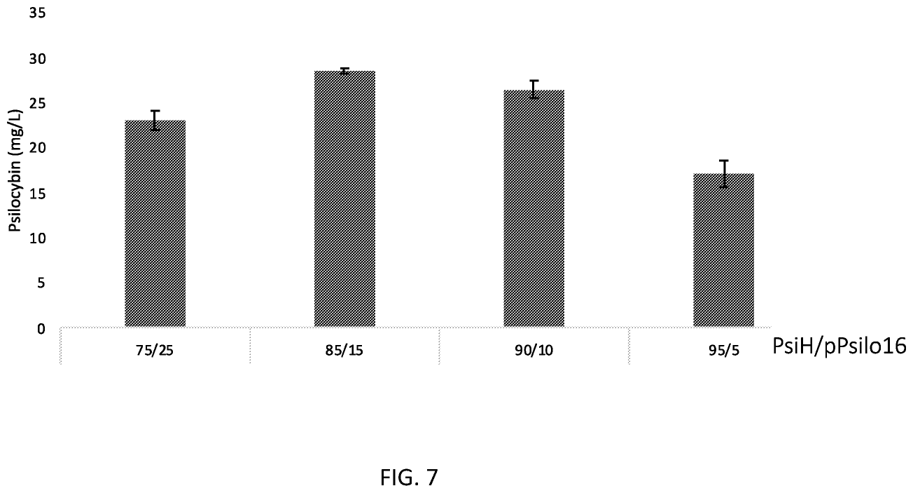

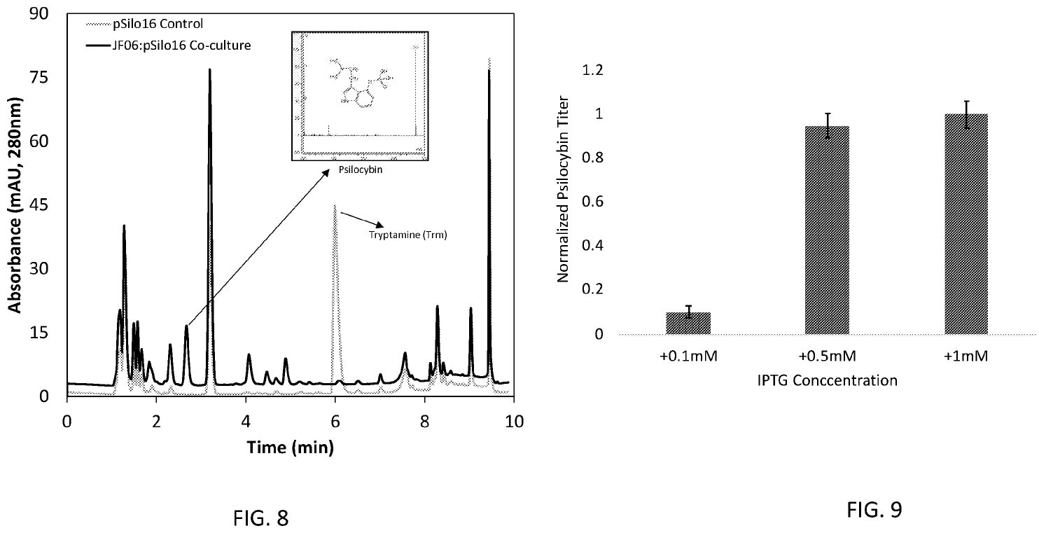

We then applied this co-culture strategy with our previously optimized pSilo16 psilocybin production strain capable of greater than 1 g/L psilocybin production with 4-hydroxyindole supplementation. The JF06-pSilo16 co-culture was then grown without indole, tryptophan, or other amino acid supplements, such that all carbon in the psilocybin product would derive directly from the glucose carbon source. We modified the range of inoculation ratios tested to further favor the JF06 strain as it was expected to be the rate limiting strain in this co-culture setup. Here we tested JF06:pSilo16 inoculation ratios of 75:25, 85:15, 90:10, and 95:5. An optimum was observed at the 85:15 ratio, with overall production of 28.5+/−0.3 mg/L of psilocybin (FIG. 7). This represents the highest de novo psilocybin production to date from a prokaryotic host. This strain was subjected to further LCMS screening to confirm the presence of psilocybin (FIG. 8). Here, the co-culture containing both strains (top trace, black) is shown to produce psilocybin (it 2.7 min, m/z 285), while the control culture containing only the psilocybin production strain, pSilo16, produces no psilocybin (bottom trace, gray). The tryptamine intermediate product can be seen to build up in the pSilo16 culture, indicating PsiD activity is present.

### Example 5: Further Optimization of De Novo Psilocybin Production

Additional screening was performed to evaluate the production of psilocybin under various process and supplement conditions. First, we looked to modify the inducer (IPTG) concentration. This is not only an important parameter for pathway performance, but IPTG is also the single most expensive media supplement. In this study we evaluated de novo psilocybin production at 0.1-, 0.5-, and 1.0-mM final concentration. Peak production was observed at the 0.5- and 1.0-mM levels with no statistical different observed between the two (p>0.05) (FIG. 9). This informs that upon scaleup of this work the ITPG concentration will be an important parameter to consider when minimizing process costs.

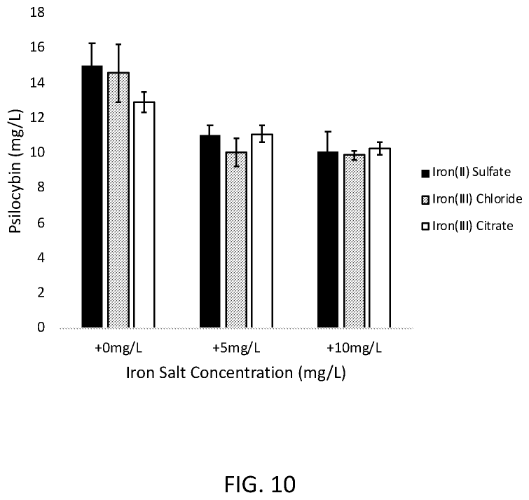

Finally, it is expected that the psiH activity is the current bottleneck limiting pathway performance. PsiH is a heme protein, containing an iron-porphyrin group that is necessary for catalytic activity (Hausjell et al., 2018). To probe the impact of iron concentration on pathway performance we supplemented the base media with an additional 5 or 10 mg/L of various iron salts (Iron (II) Sulfate, Iron (III) Chloride, or Iron (III) Citrate). The base media contains 1.5 mg/L (9.9 uM) of FeSO₄. Across all conditions, no preference for a specific iron salt was observed and the best production was achieved with no additional iron (FIG. 10). This indicates that the iron levels provided by the base media are sufficient for PsiH activity at this scale, but as these systems are scaled up, further studies may be justified.

### Example 6: Evaluation of Substrate Flexibility in Psilocybin Biosynthetic Pathway

Now that a proof-of-principle de novo production psilocybin production system had been developed, we looked to leverage these novel biosynthetic capabilities to produce a range of non-natural tryptamine products through supplementation of substituted indole derivatives. Having identified 45 indole derivatives, we first set out to evaluate substrate promiscuity of E. coli's native tryptophan synthase (TrpB) and the first step in the psilocybin biosynthetic pathway, PsiD, as described above. We then followed with the application of the JF06/pSilo16 co-culture approach as described above to facilitate the biosynthesis of various non-natural psilocybin derivatives leveraging the native promiscuity of the developed biosynthesis pathway.

The substrate flexibility of TrpB, PsiD, PsiH, PsiK, and PsiM were evaluated by the supplementation of 45 substituted indole derivatives, comprised of 10 monosubstituted chemical moieties, across 6 chemical positions. In addition, select combinations were evaluated under differing media conditions (with or without serine), process conditions (with or without substrate delay), and genetic backgrounds (promoter optimization for PsiD expression). Results from each trial are presented below and represent the most complete analysis of substrate flexibility for the psilocybin biosynthesis pathway performed to date.

### Example 7: Evaluation of TrpB Substrate Flexibility without Supplemental Serine

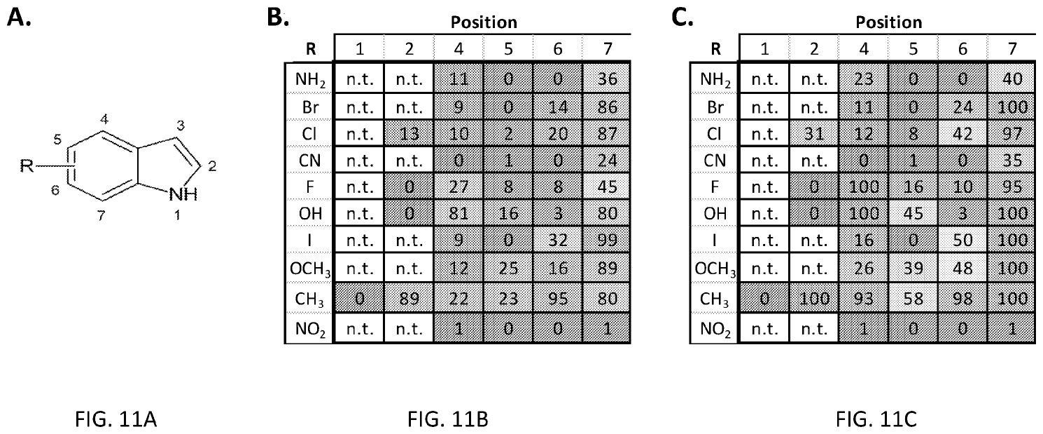

A heatmap summary of the below data can be seen in FIG. 11B.

Nine substituted indoles processed through TrpB without serine at a high proficiency (>65%); 7-bromoindole, 7-chloroindole, 4-hydroxyindole, 7-iodoindole, 7-hydroxyindole, 7-methoxyindole, 2-methylindole, 6-methylindole, and 7-methylindole.

Twenty substituted indoles processed through TrpB without serine at moderate (35-65%) or low proficiency (5-35%); 4-aminoindole, 7-aminoindole, 4-bromoindole, 6-bromoindole, 2-chloroindole, 4-chloroindole, 6-chloroindole, 7-cyanoindole, 4-fluoroindole, 5-fluoroindole, 6-fluoroindole, 7-fluoroindole, 5-hydroxyindole, 4-iodoindole, 6-iodoindole, 4-methoxyindole, 5-methoxyindole, 6-methoxyindole, 4-methylindole, and 5-methylindole.

Sixteen substituted indoles processed through TrpB without serine at poor (>0-5%) to no proficiency (0%); 5-aminoindole, 6-aminoindole, 5-bromoindole, 5-chloroindole, 4-cyanoindole, 5-cyanoindole, 6-cyanoindole, 2-fluoroindole, 2-hydroxyindole, 6-hydroxyindole, 5-iodoindole, 1-methylindole, 4-nitroindole, 5-nitroindole, 6-nitroindole, and 7-nitroindole.

### Example 8: Evaluation of TrpB Substrate Flexibility with Supplemental Serine

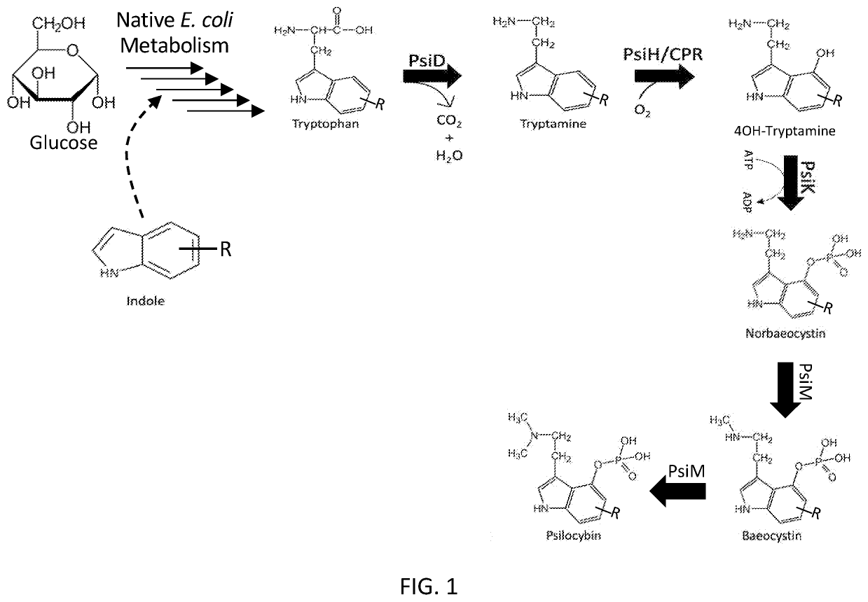

A heatmap summary of the below data can be seen in FIG. 1 IC.

Eight substituted indoles completely processed through TrpB with supplemental serine (100%); 2-methyindole, 7-bromoindole, 4-fluoroindole, 4-hydroxyindole, 7-hydroxyindole, 7-iodoindole, 7-methoxyindole, and 7-methylindole. Of these, 4-fluoroindole stood out with the greatest enhancement as compared to the without indole condition, increasing from 27% to 100% conversion.

Four substituted indoles processed through TrpB with supplemental serine at a high proficiency (>65%); 7-chloroindole, 7-fluoroindole, 4-methylindole, and 6-methylindole. Of those only 7-fluoroindole and 4-methylindole demonstrated significant enhancement over the without indole condition increasing from 45% and 22% to 95% and 93%, respectively.

Eight substituted indoles processed through TrpB with supplemental serine at a moderate proficiency; 7-aminoindole, 6-chloroindole, 7-cyanoindole, 5-hydroxyindole, 6-iodoindole, 5-methoxyindole, 6-methoxyindole, and 5-methylindole.

Ten substituted indoles processed through TrpB with supplemental serine at a low proficiency; 4-aminoindole, 4-bromoindole, 6-bromoindole, 2-chloroindole, 4-chloroindole, 5-chloroindole, 5-fluoroindole, 6-fluoroindole, 4-iodoindole, and 4-methoxyindole.

Fifteen substituted indoles processed through TrpB with serine at little to no proficiency (0-5%); 5-aminoindole, 6-aminoindole, 5-bromoindole, 4-cyanoindole, 5-cyanoindole, 6-cyanoindole, 2-fluoroindole, 2-hydroxyindole, 6-hydroxyindole, 6-iodoindole, 1-methyindole, 4-nitroindole, 5-nitroindole, 6-nitroindole, and 7-nitroindole.

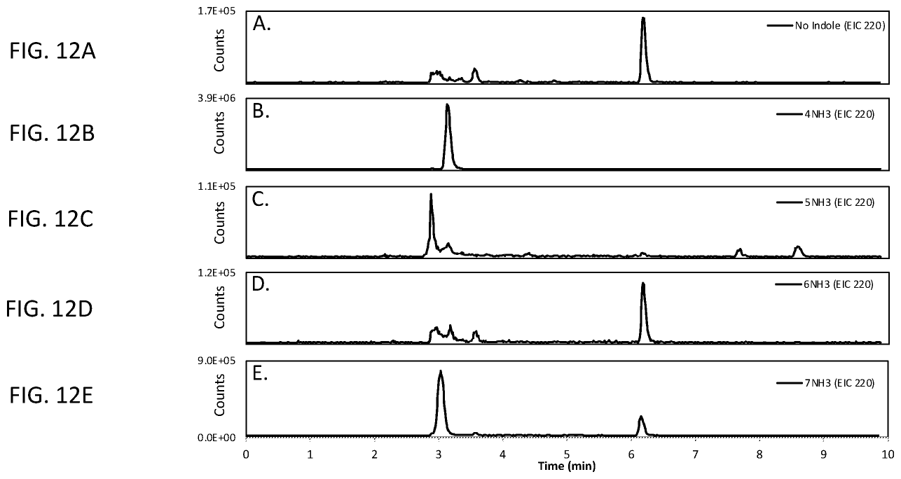

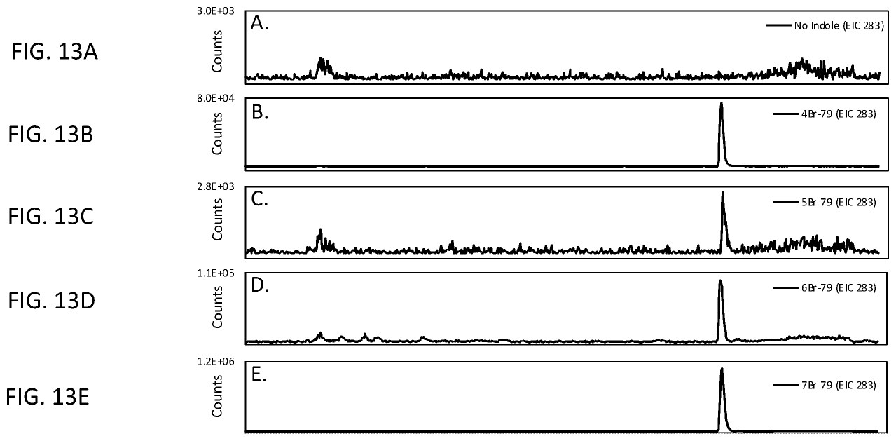

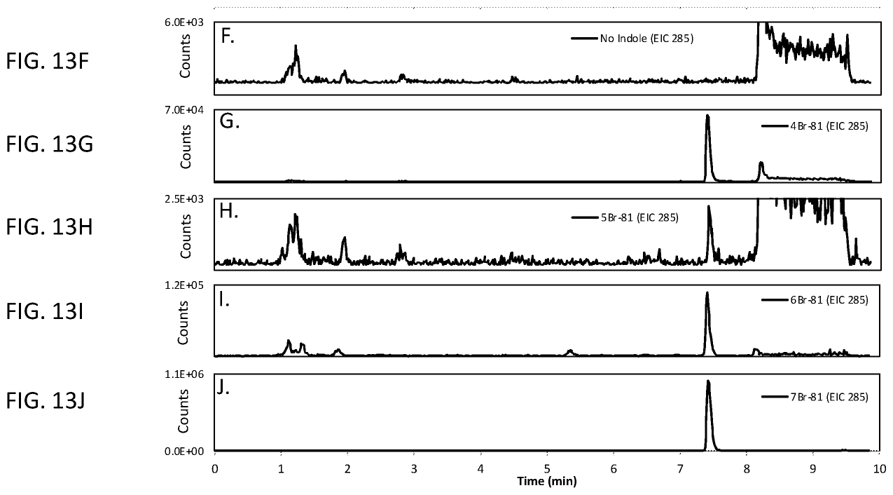

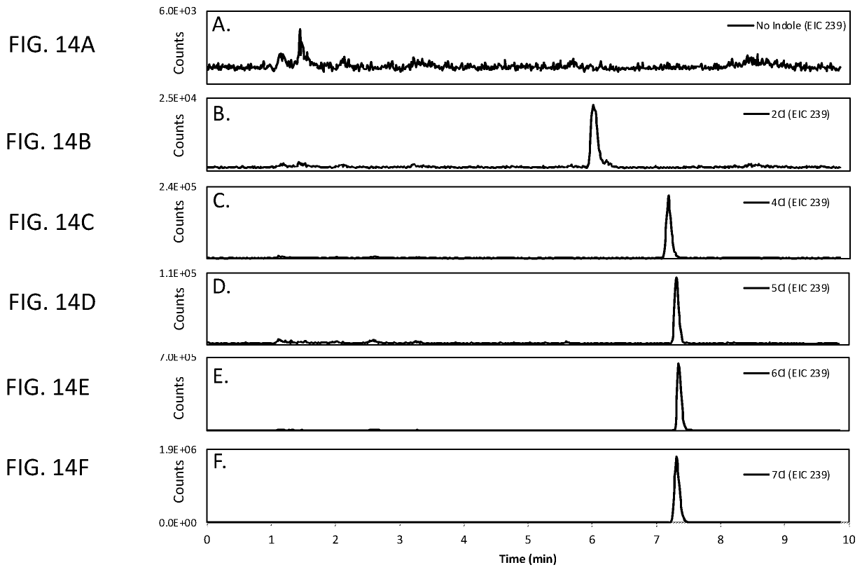

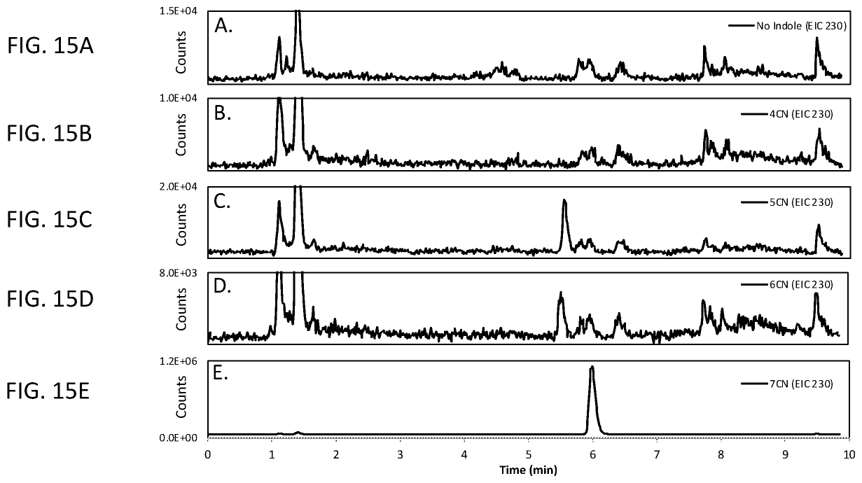

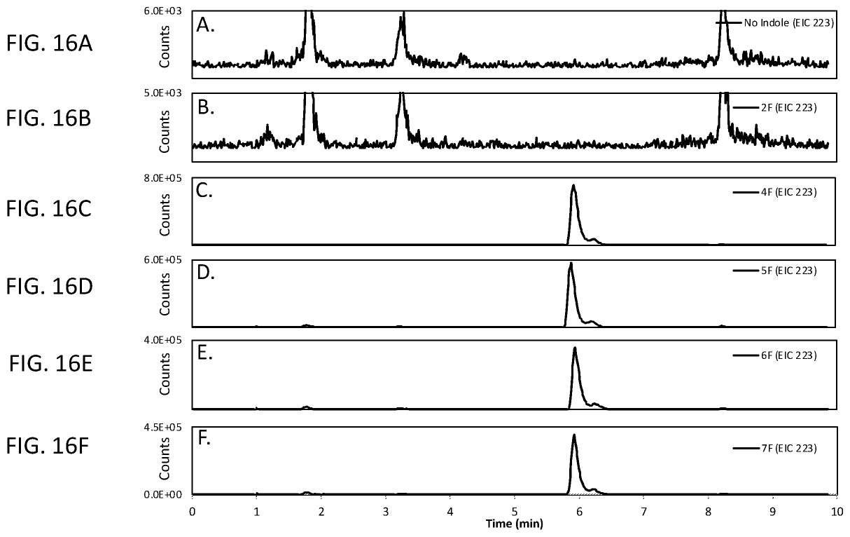

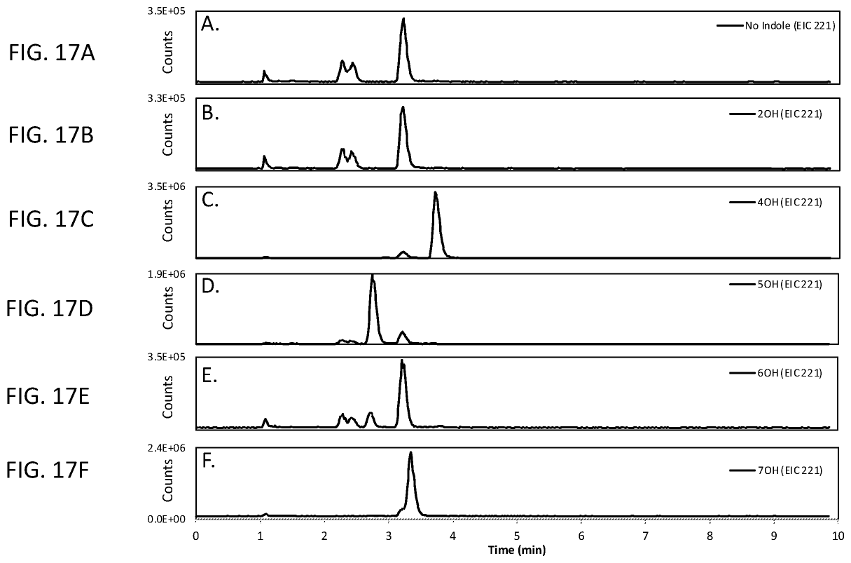

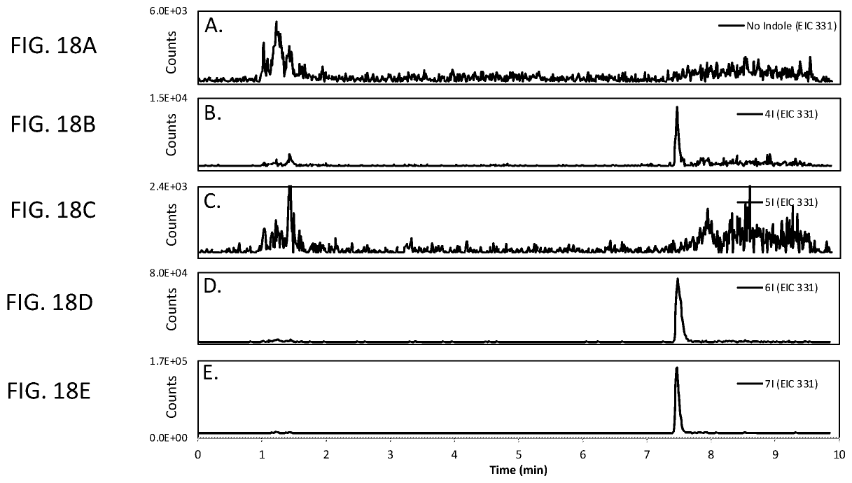

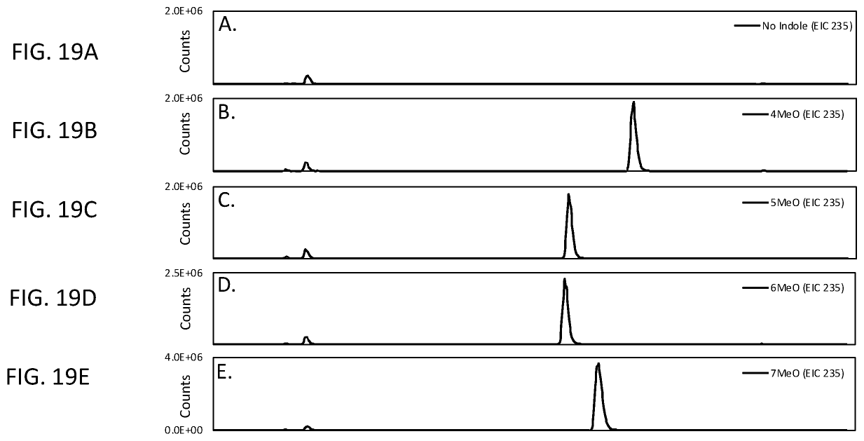

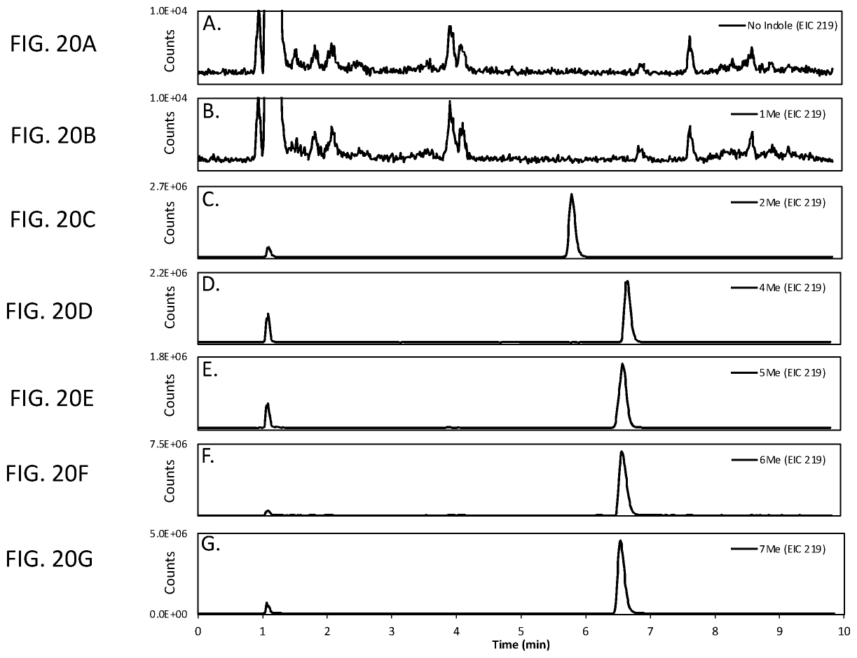

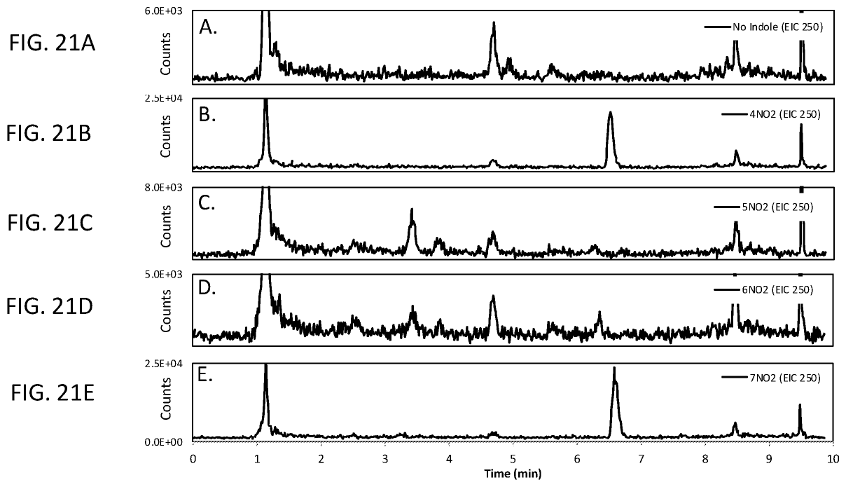

Across the full range of 45 substrates tested, the addition of serine to the media resulted in enhanced activity of TrpB, leading to the detection of 34 substituted tryptophans. The exceptions to this trend are the indoles that showed no or trace conversion under the without serine condition as these continued to demonstrate no or trace production, resulting in no substantial change in effective activity. Indoles substituted at the 4 and 7 positions generally processed with higher efficiency using native E. coli TrpS in vivo. This is in contrast to results obtained by the Goss lab, who reported the opposite trend with haloindoles, using cell-free lysate containing S. enterica TrpS (Goss and Newill, 2006). TrpB substrate yield results are shown in FIGS. 11A-11C and LCMS extracted ion chromatographs for all tryptophan substrates tested can be found in FIGS. 12-21.

### Example 9: Evaluation of PsiD Substrate Flexibility

PsiD also demonstrated significant functional substrate flexibility with many substituted tryptophans produced by TrpB also undergoing decarboxylation catalyzed by PsiD to produce substituted tryptamines. PsiD substrate flexibility was evaluated jointly in vivo with TrpB, such that if indoles were not sufficiently processed by TrpB, then PsiD activity could not be evaluated. PsiD proficiency is defined as the fraction of substituted tryptamine produced over the sum of substituted tryptophan remaining and substituted tryptamine produced. This metric highlights the conversion of available substrate, but should not be interpreted as final titer as the production of substituted tryptophans by TrpB does vary widely, as demonstrated above.

Four substituted indoles resulted in substituted tryptophans that processed through PsiD at a high proficiency (>65%); 4-aminoindole, 4-hydroxyindole, 6-hydroxyindole, and 4-methylindole. Of these 4-hydroxyindole and 4-methylindole also processed through TrpB alone with a high proficiency (100% and 93%, respectively), while 4-aminoindole and 6-hydroxyindole processed through TrpB with 23% and 3% proficiency, respectively. This indicates that the latter two products likely experienced a bottleneck at the TrpB biosynthetic step. Production of 6-hydroxytryptophan and 6-hydroxytryptamine using Psilocybe cubensis TrpS and PsiD in vitro was not observed previously (Hoffmeister 2018).

Eight substituted indoles processed through PsiD at low proficiency (5-35%); 4-fluoroindole, 5-fluoroindole, 6-fluoroindole, 7-fluoroindole, 5-hydroxyindole, 7-hydroxyindole, 5-methoxyindole, and 5-methylindole. Of these 4-fluoroindole, 7-fluoroindole, and 7-hydroxyindole. Of these 7-hydroxyindole, 4-fluoroindole, and 7-fluoroindole processed through TrpB with a high proficiency (>65%) with the others likely experiencing a bottleneck at the TrpB biosynthetic step.

Six substituted indoles processed through PsiD at a poor proficiency (1-5%); 7-aminoindole, 7-bromoindole, 7-chloroindole, 6-methoxyindole, 7-methoxyindole, 6-methylindole, and, 7-methylindole.

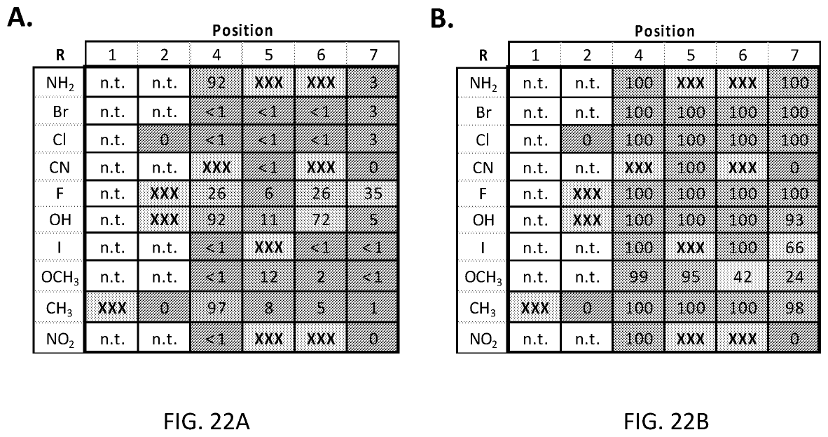

The remaining twenty-seven substituted indoles processed through PsiD at little to no proficiency with the T7 consensus promoter controlling expression of PsiD. This low performance is due in part due to limited synthesis of substituted tryptophans due to low TrpB activity towards many indoles in this group, including a group of eleven indoles which showed no processing through TrpB. However, certain indoles, such as 2-methylindole, showed high conversion by TrpB, but no tryptamine product could be detected in the presence of PsiD. The broad range of substrates tested and corresponding broad range of activities observed may give some insight to structure-function relationships of these two important enzymes. Results for all PsiD substrate flexibility screening data under consensus T7 control can be found in FIG. 22A.

In an attempt to further enhance PsiD performance, two previously published and genetically optimized strains for norbaeocystin (pNor) and psilocybin (pSilo16) production were also used to assess production of substituted tryptamines. These optimized strains contained additional pathway enzymes (psiK and psiM), but these enzymes have been documented to require a hydroxy or phosphoroxy group at the 4-position, respectively. Due to this, they are not expected to act on many of the selected substrates, but the highly optimized nature of these strains, will allow psiD activity to be evaluated in an environment with minimized metabolic burden. Upon evaluation, these strains showed enhanced tryptamine peaks, including thirteen products that were not previously observed as indicated by the ‘<1’ designation in FIG. 22A. These results motivated the transcriptional optimization of PsiD constructs in an attempt to enhance the diversity of chemical derivatives that could be produced.

### Example 10: Transcriptional Library Screening Enhances Strain Performance

A library of 9 varying transcriptional configurations was screened for 29 of the substituted indoles that showed activity through TrpB. This transcriptional library consisted of 5 IPTG-inducible T7 mutant promoters (G6, H9, H10, T7, and C4), 2 constitutive promoters (XylA and GAP), and 2 reduced expression vector backbones with the T7 consensus promoter (pACM4 and pCDM4). Results of promoter library testing is presented FIG. 22B.

Across the board, yields for each indole were greatly increased with promoter optimization. Interestingly, the promoter identified for maximal production for each indole supplement varied widely when all other conditions remained constant. This indicates that promoter optimization will need to be performed for each indole supplement separately to develop strains for maximal derivatized tryptamine production. It is also hypothesized that this optimization strategy will be necessary to realize full pathway performance upon extension of this pathway towards the production of other more complex tryptamines such as derivatives of psilocybin, N,N-dimethyltryptamine (DMT), and norbaeocystin.

HPLC peak areas were used for analysis of promoter library testing results since reaction products were identical, even if absolute concentrations cannot be determined or compared across differing indole trials. In general, the T7, pCDM4-T7, and pACM4-T7 conditions resulted the lowest yields, while the H9, H10, and C4 promoters resulted in the highest yields, with a few exceptions. The weak G6 promoter demonstrated the highest variability across various indole trials with some indoles showing G6-based production equivalent to the best strains, while for others it represented one of the worst performing configurations. The constitutive promoters pXylA and pXPA frequently resulted in high to moderate titers.

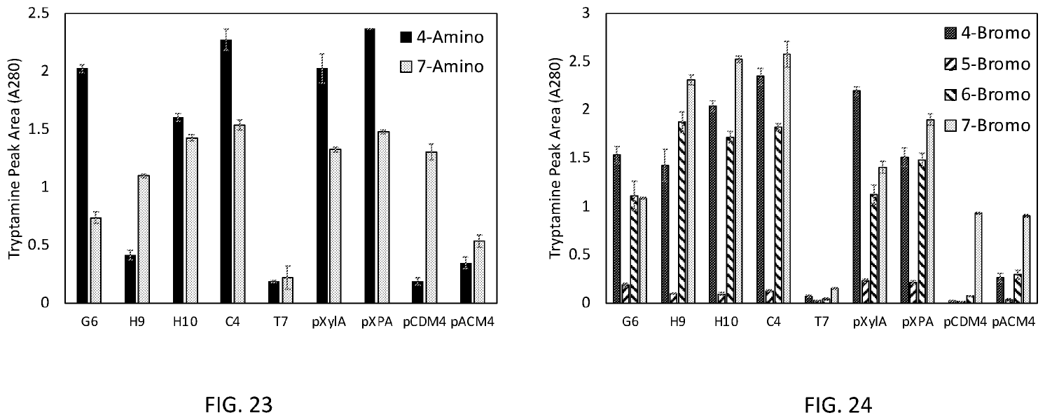

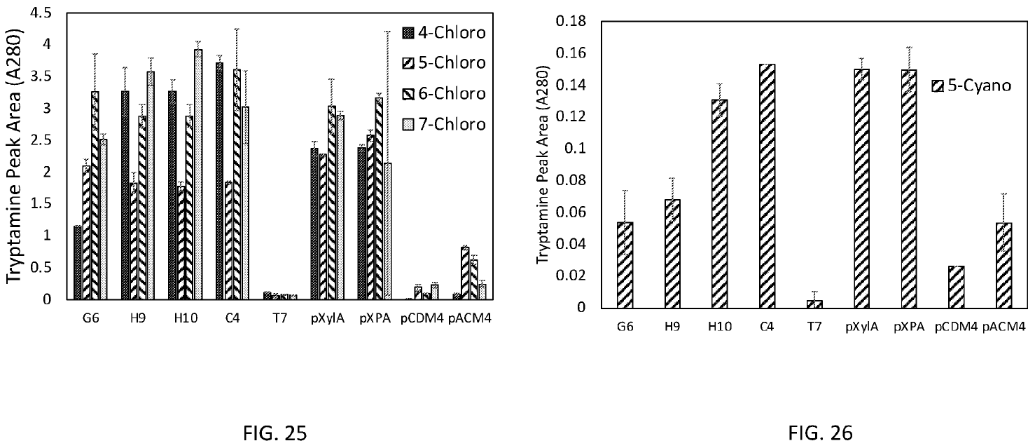

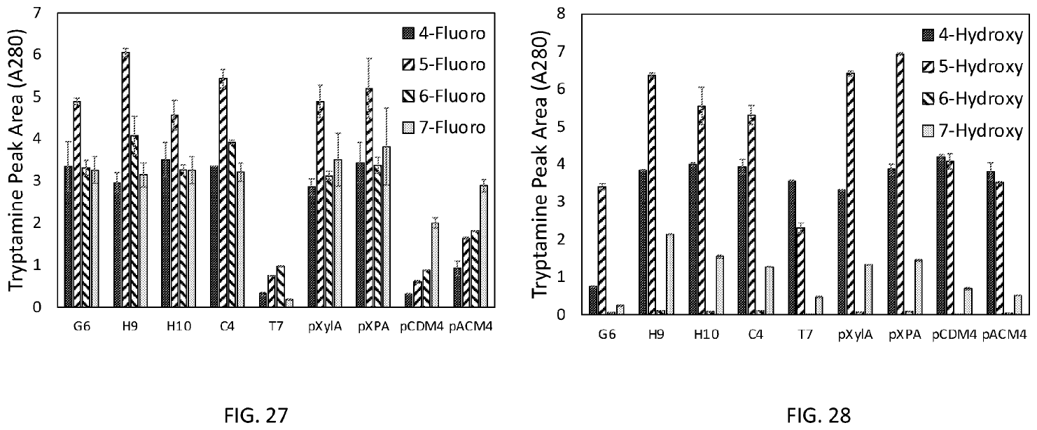

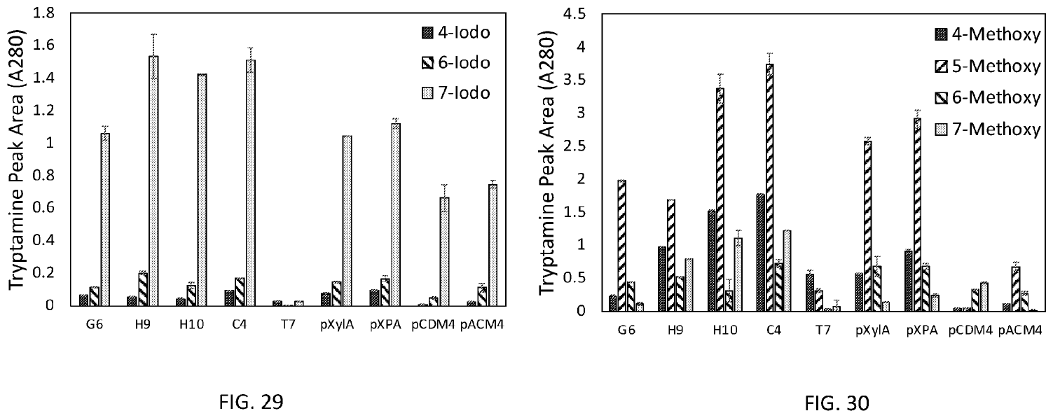

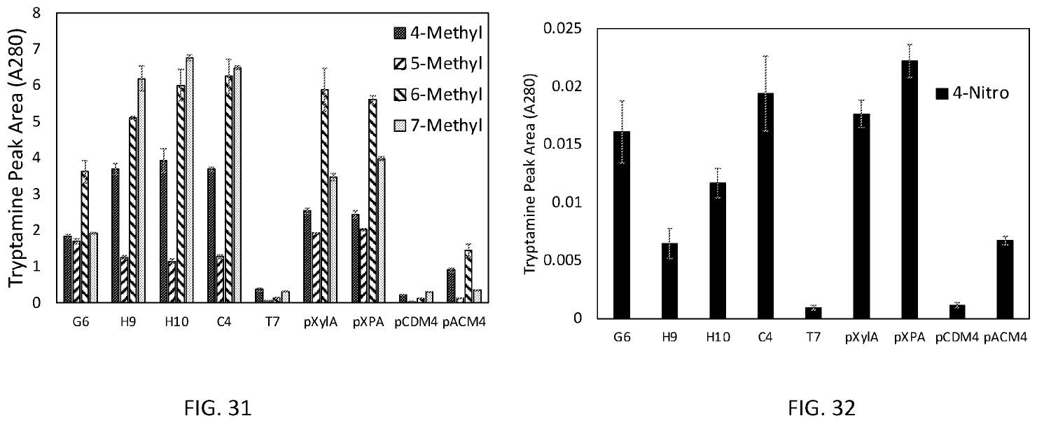

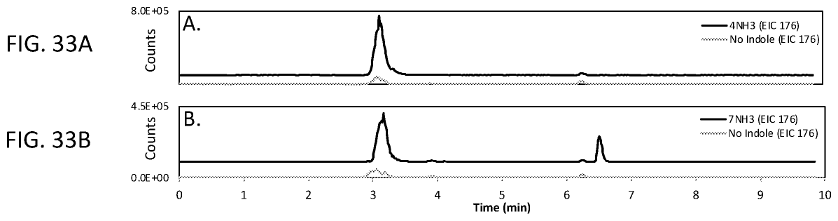

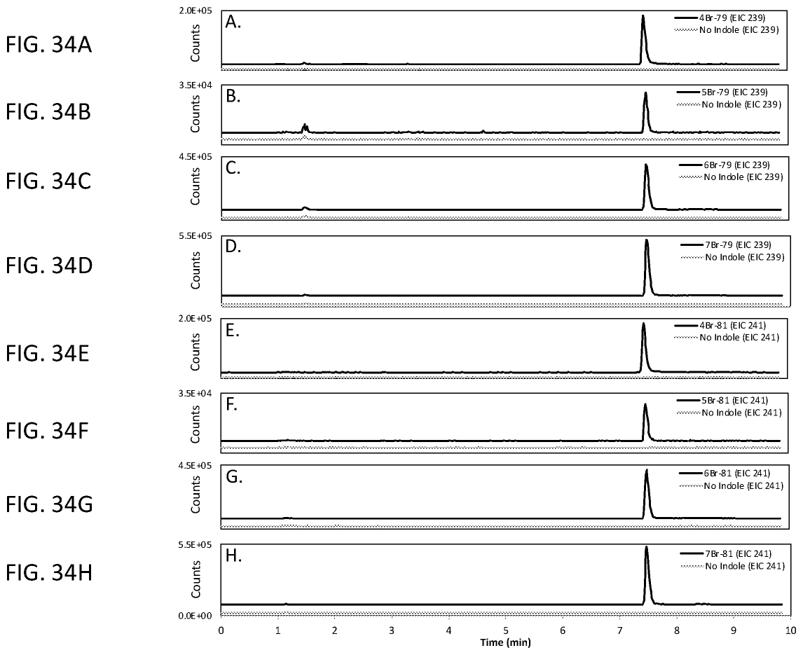

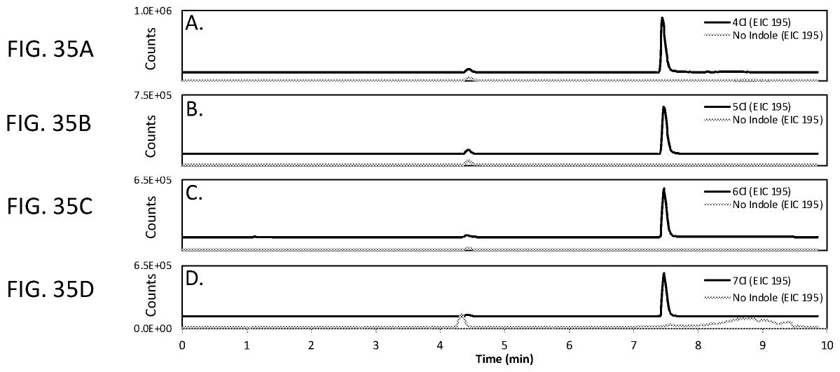

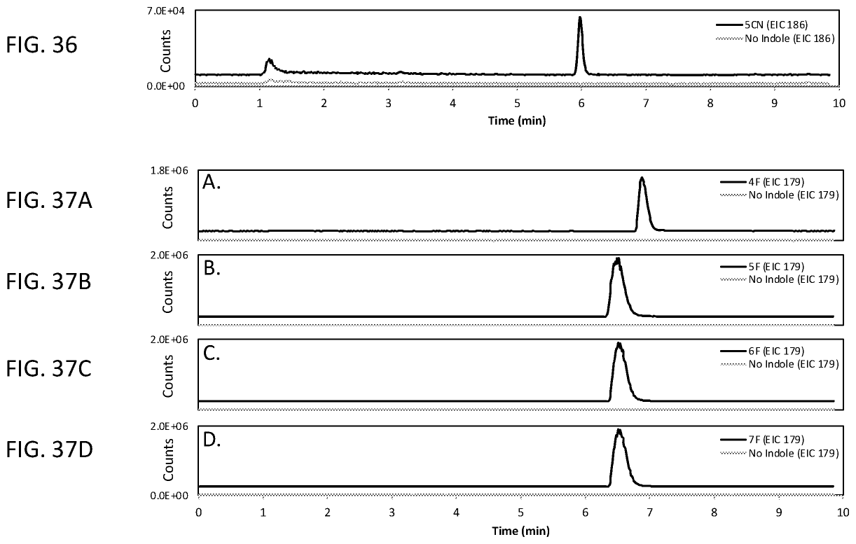

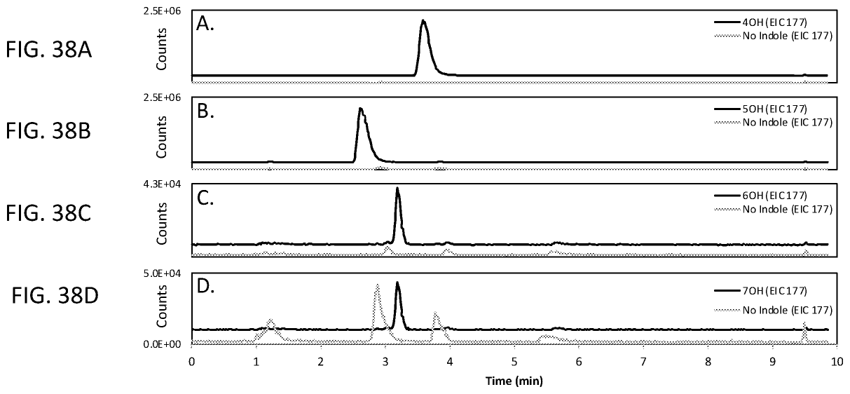

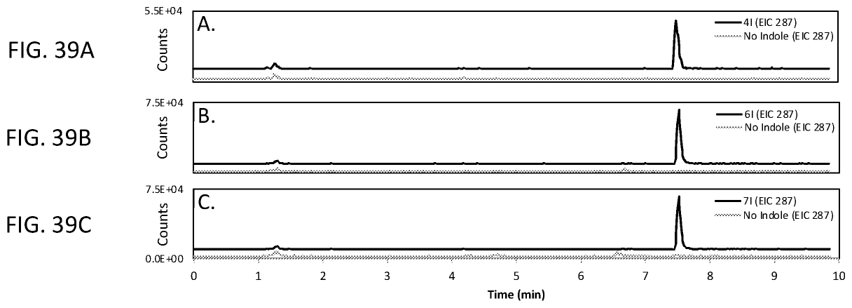

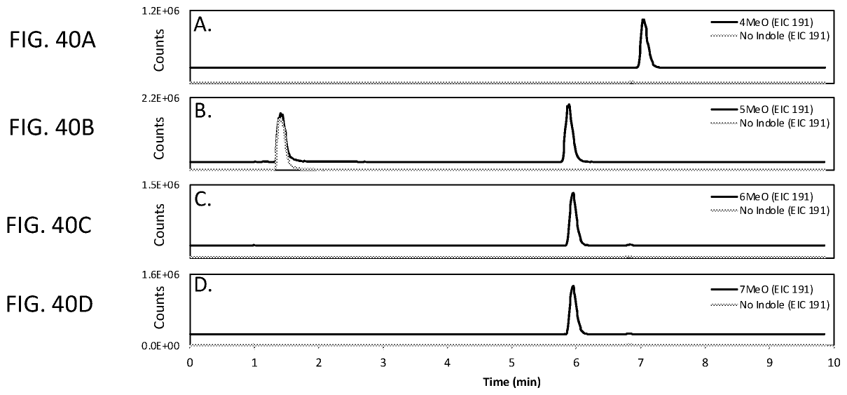

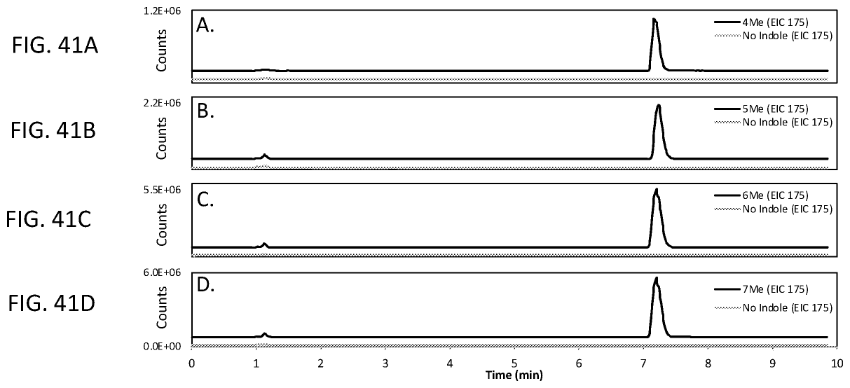

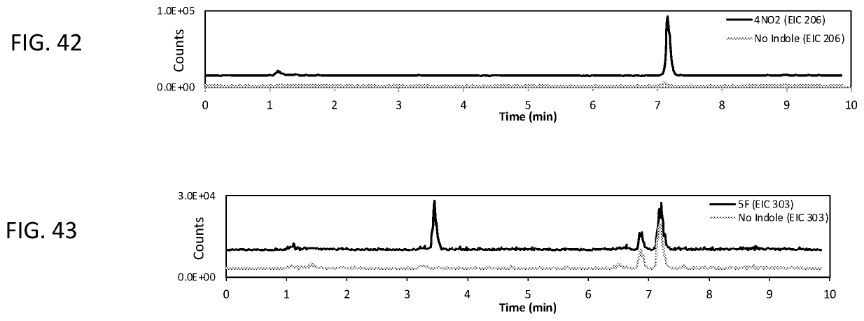

The PsiD promoter optimization study resulted in the production of 13 tryptamine derivatives that were previously not definitively detected from the original study utilizing the consensus T7 promoter. This raises the number of verified PsiD substrates to 31, not including its natural substrate, tryptophan. This is out of the 34 possible substrates that passed through TrpB. Notably, we did detect the formation of 5-bromotryptamine, whose precursor, 5-bromotryptophan, was not previously observed in the TrpB alone study. Complete library screening data can be found in FIGS. 23-32, and LCMS extracted ion chromatographs for all tryptamines produced can be found in FIGS. 33-42.

### Example 11: Co-Culture Application for Production of Non-Natural Psilocybin Derivatives and Intermediate Products

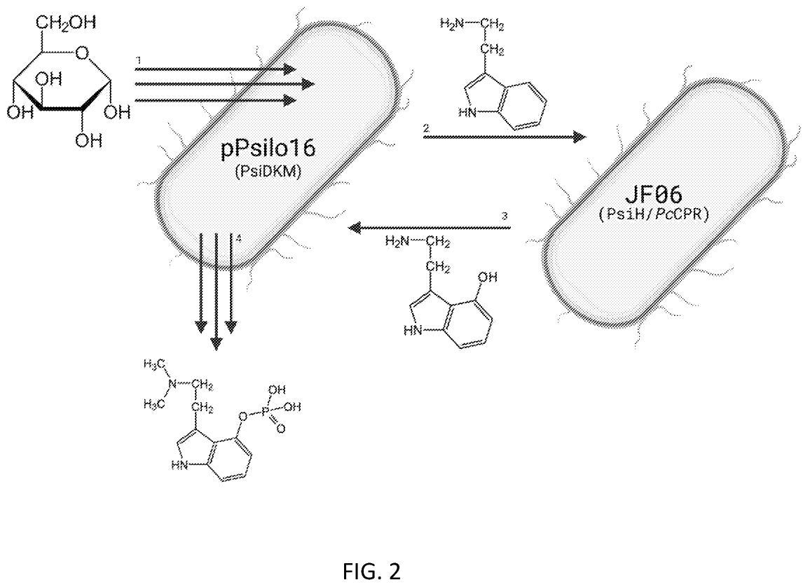

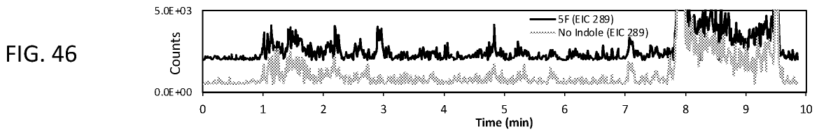

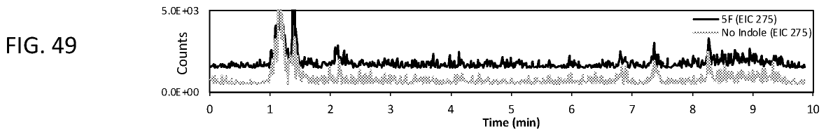

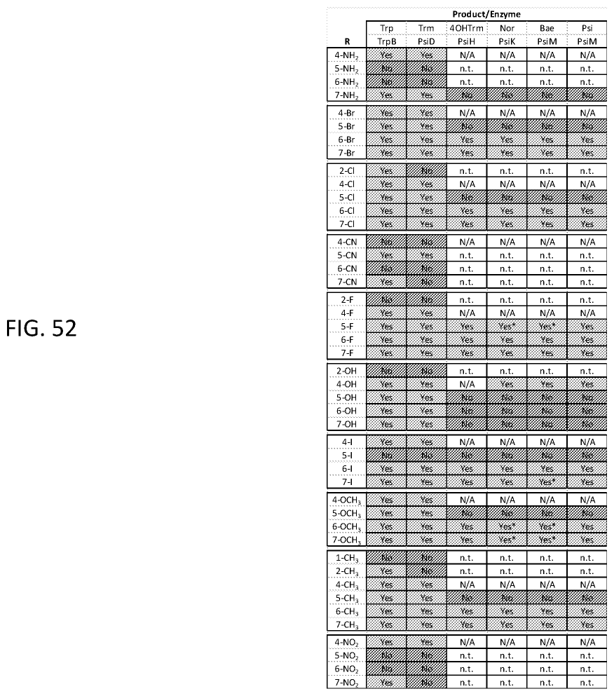

To realize the full biosynthesis of non-natural psilocybin derivatives a microbial co-culture approach was used as a proof of principle. Combining a previously optimized psilocybin production strain (pSilo16), expressing PsiD, PsiK, and PsiM with a recently developed strain, JF06, capable of functional expression of the P450 hydroxylase, PsiH, and its reductase partner, CPR, we were able to achieve the biosynthesis of thirteen non-natural psilocybin derivatives (FIGS. 43-45). In this system, the substituted indole is converted to a substituted tryptamine as reported above. This substituted tryptamine is then used as a substrate for PsiH, resulting in hydroxylation at the 4-position before further conversion by PsiK and PsiM in the original psilocybin production strain. This co-culture process involves a minimum of two mass transport processes. First the transfer of substituted tryptamine from the psilocybin production strain containing psiD to the hydroxylase strain containing psiH, followed by the resulting transfer of 4-hydroxy-substituted tryptamines back to the psilocybin production strain (FIG. 2). The application of this process successfully produced low amounts of thirteen psilocybin derivatives, providing proof-of-principle for the use of the natural psilocybin biosynthetic pathway to produce a range of non-natural psilocybin derivatives, including; 6-bromopsilocybin, 7-bromopsilocybin, 6-chloropsilocybin, 7-chloropsilocybin, 5-fluoropsilocybin, 6-fluoropsilocybin, 7-fluoropsilocybin, 6-iodopsilocybin, 7-iodopsilocybin, 6-methoxypsilocybin, 7-methoxypsilocybin, 6-methylpsilocybin and 7-methylpsilocybin. Mass spectra extracted ion chromatographs supporting the production of non-natural psilocybin derivatives are presented in FIGS. 43-45, non-natural baeocystin derivatives are presented in FIGS. 46-48, and non-natural norbaeocystin derivatives are presented in FIGS. 49-51. A summary of all pathway promiscuity can be found in FIG. 52.

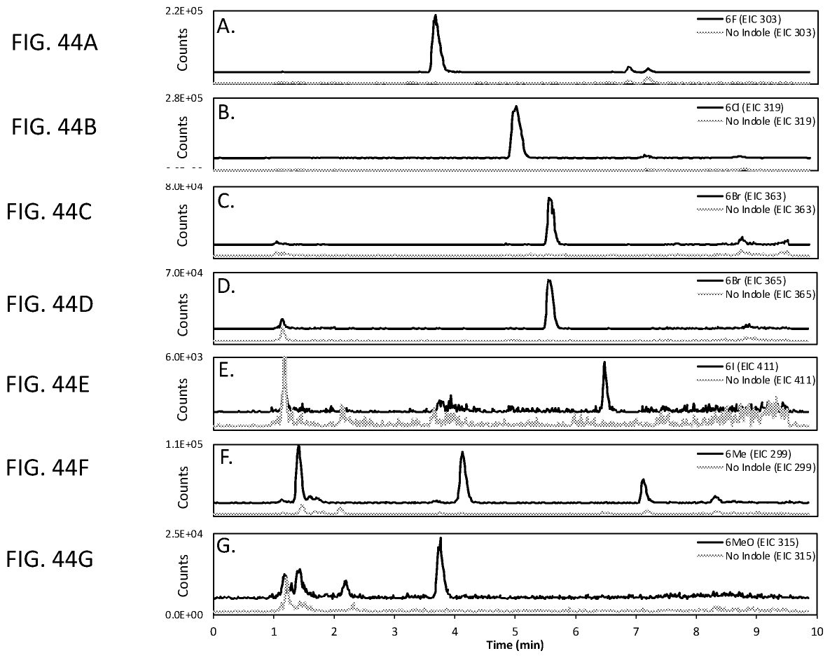

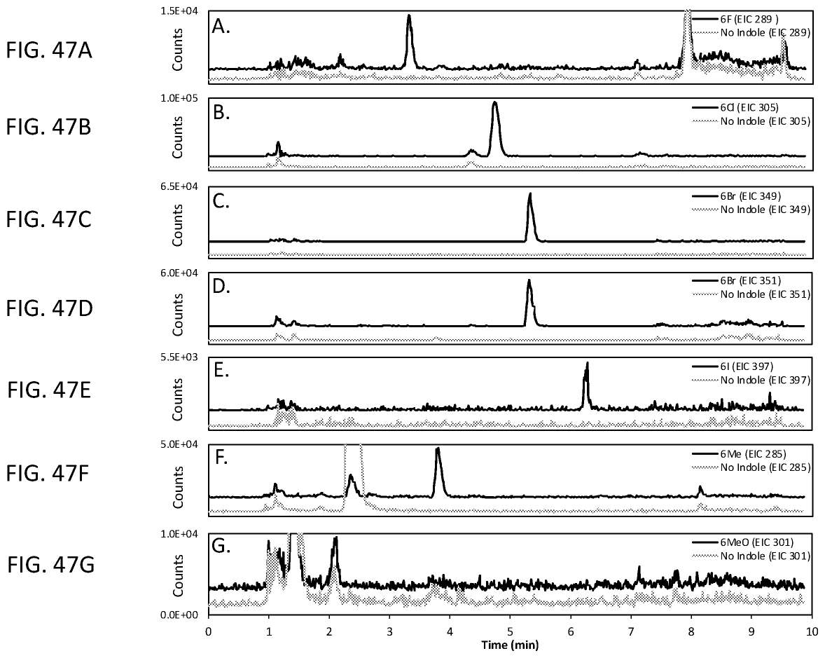

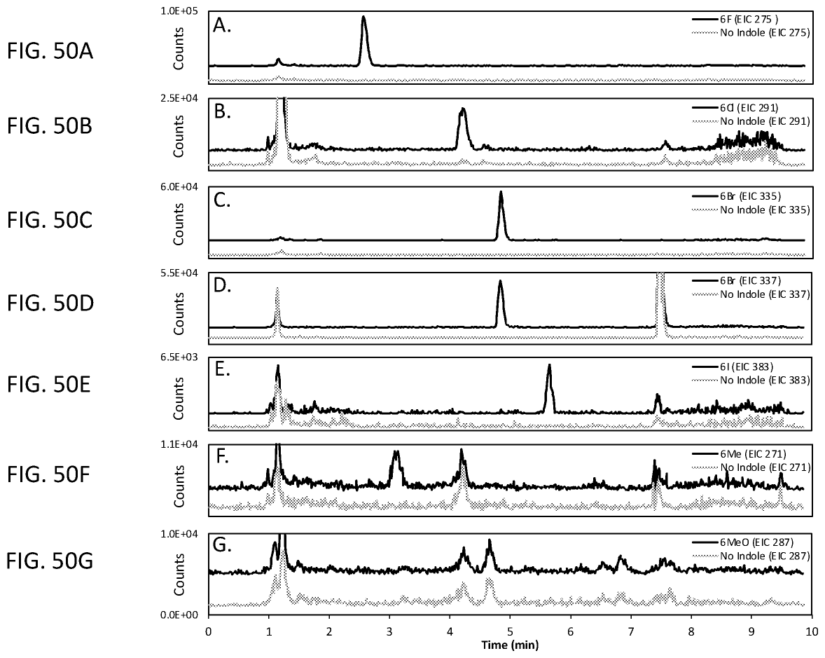

Supplementation of 6-bromoindole produced two new A280 product peaks on the HPLC chromatogram at 5.2 and 5.4 minutes. Since bromine has two stable isotopes in nearly equal abundance, ⁷⁹Br and ⁸¹Br, two masses are expected for each product. The peak at 5.2 minutes had masses of 349 and 351, which correlated with the expected masses for 6-bromobaeocystin (FIGS. 47C-47D), while the peak at 5.4 minutes had masses of 363 and 365, which correlated with the expected masses for 6-bromopsilocybin (FIGS. 44C-44D). Accumulation of 6-bromonorbaeocystin was not observed in the 6-bromopsilocybin production study, however when the psilocybin production strain, pSilo16, was replaced with the norbaeocystin production strain, pNor, 6-bromonorbaeocystin was observed as a terminal product (FIGS. 50C-50D).

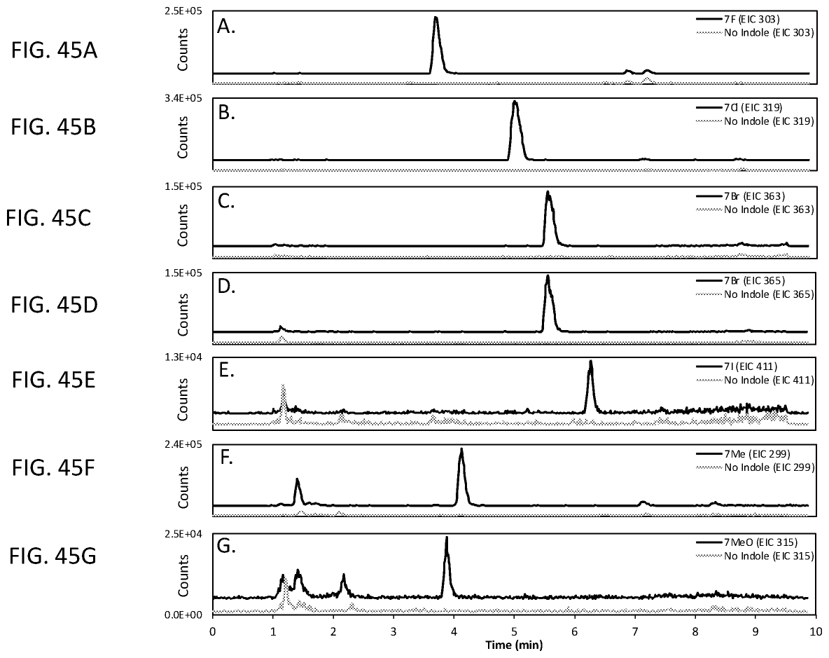

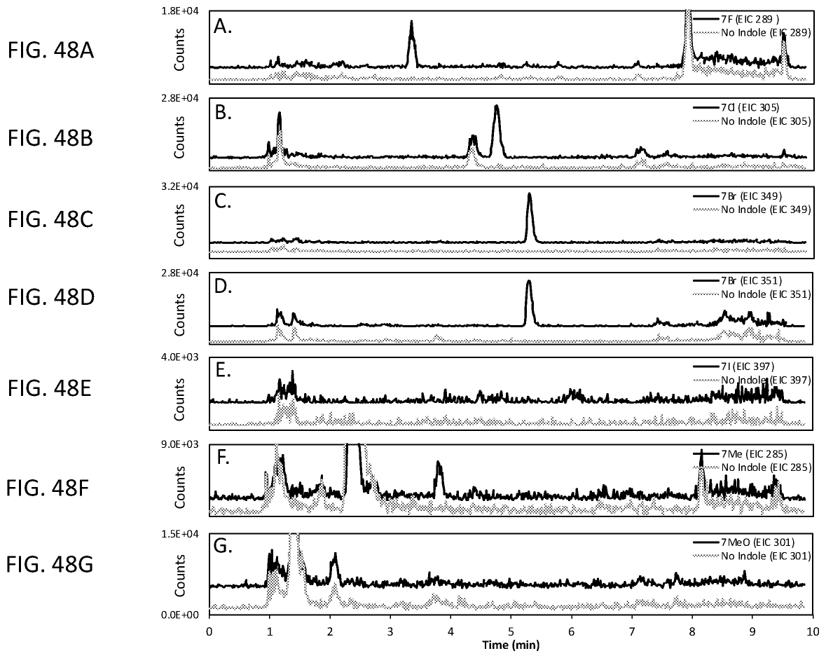

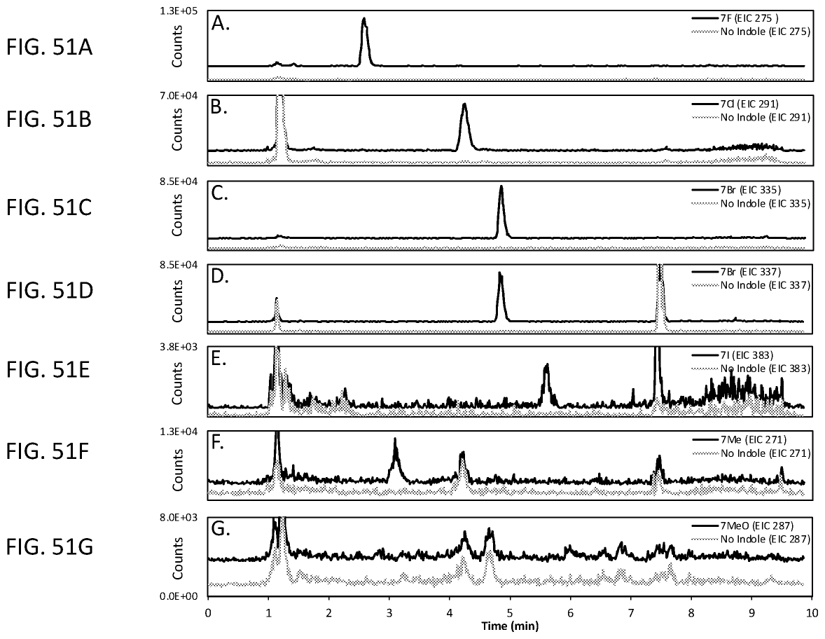

Supplementation of 7-bromoindole produced two new A280 product peaks on the HPLC chromatogram at 5.2 and 5.4 minutes. The smaller peak at 5.2 minutes had masses of 349 and 351, which correlated with the expected masses for 7-bromobaeocystin (FIGS. 48C-48D), while the peak at 5.4 minutes had masses of 363 and 365, which correlated with the expected masses of 7-bromopsilocybin (FIGS. 45C-45D). Accumulation of 7-bromonorbaeocystin was not observed in the 7-bromopsilocybin production study, however when the psilocybin production strain, pSilo16, was replaced with the norbaeocystin production strain, pNor, 7-bromonorbaeocystin was observed as a terminal product (FIGS. 51C-51D).

Supplementation of 6-chloroindole produced two new A280 product peaks on the HPLC chromatogram at 4.7 and 4.9 minutes. The peak at 4.7 minutes had a mass of 305, which correlated with the expected mass for 6-chlorolbaeocystin (FIG. 47B), while the peak at 4.9 minutes had a mass of 319, which correlated with the expected mass for 6-chloropsilocybin (FIG. 44B). Accumulation of 6-chloronorbaeocystin was not observed in the 6-chloropsilocybin production study, however when the psilocybin production strain, pSilo16, was replaced with the norbaeocystin production strain, pNor, 6-chloronorbaeocystin was observed as a terminal product (FIG. 50B).

Supplementation of 7-chloroindole produced two new A280 product peaks on the HPLC chromatogram at 4.7 and 4.9 minutes. The peak at 4.7 minutes had a mass of 305, which correlated with the expected mass for 7-chlorobaeocystin (FIG. 48B), while the peak at 4.9 minutes had a mass of 319, which correlated with the expected mass of 7-chloropsilocybin (FIG. 45B). Accumulation of 7-chloronorbaeocystin was not observed in the 7-chloropsilocybin production study, however when the psilocybin production strain, pSilo16, was replaced with the norbaeocystin production strain, pNor, 7-chloronorbaeocystin was observed as a terminal product (FIG. 51B).

Supplementation of 5-fluoroindole produced one new A280 product peak on the HPLC chromatogram at 3.3 minutes. The peak at 3.3 minutes had a mass of 303, which correlated with the expected mass of 5-fluoropsilocybin (FIG. 43). The precursor compounds 5-fluoronorbaeocystin, 5-fluorobaeocystin were not detected (FIGS. 49 and 46, respectively), but were assumed to be present as intermediate products in the biosynthetic pathway.

Supplementation of 6-fluoroindole produced two new A280 product peaks on the HPLC chromatogram at 3.1 and 3.5 minutes. The peak at 3.1 minutes had a mass of 289, which correlated with the expected mass for 6-fluorobaeocystin (FIG. 47A), while the peak at 3.5 minutes had a mass of 303, which correlated with the expected mass for 6-fluoropsilocybin (FIG. 44A). Accumulation of 6-fluoronorbaeocystin was not observed in the 6-fluoropsilocybin production study, however when the psilocybin production strain, pSilo16, was replaced with the norbaeocystin production strain, pNor, 6-fluoronorbaeocystin was observed as a terminal product (FIG. 50A).

Supplementation of 7-fluoroindole produced two new A280 product peaks on the HPLC chromatogram at 3.2 and 3.6 minutes. The peak at 3.2 minutes had a mass of 289, which correlated with the expected mass of 7-fluorobaeocystin (FIG. 48A), while the peak at 3.6 minutes had a mass of 303, which correlated with the expected mass for 7-fluoropsilocybin (FIG. 45A). Accumulation of 7-fluoronorbaeocystin was not observed in the 7-fluoropsilocybin production study, however when the psilocybin production strain, pSilo16, was replaced with the norbaeocystin production strain, pNor, 7-fluoronorbaeocystin was observed as a terminal product (FIG. 51A).

Supplementation of 6-iodoindole produced three new A280 product peaks on the HPLC chromatogram at 5.6, 6.2, and 6.4 minutes. These peaks had masses of 382, 396, and 410, respectively, which correlated with the expected mass of 6-iodonorbaeocystin, 6-iodobaeocystin, and 6-iodopsilocybin (FIGS. 50E, 47E, 44E, respectively).

Supplementation of 7-Iodoindole produced a new A280 product peak on the HPLC chromatogram at 6.2 minutes. The peak at 6.2 minutes had a mass of 410, which correlated with the expected mass for 7-iodopsilocybin (FIG. 45E). The precursor compounds 7-iodonorbaeocystin, 7-iodobaeocystin were not observed in the 7-iodopsilocybin production study, however when the psilocybin production strain, pSilo16, was replaced with the norbaeocystin production strain, pNor, 7-iodonorbaeocystin was observed as a terminal product (FIG. 51E).

Supplementation of 6-methoxyindole produced one new A280 peak on the HPLC chromatogram at 3.7 minutes. The small peak at 3.7 minutes had a mass of 315, which correlated with the expected mass of 6-methoxypsilocybin (FIG. 44G). The precursor compounds 6-methoxynorbaeocystin, 6-methoxybaeocystin were not detected, but were assumed to be present as intermediate products in the biosynthetic pathway.

Supplementation of 7-methoxyindole produced one new A280 peak on the HPLC chromatogram at 3.8 minutes. The small peak at 3.8 minutes had a mass of 315, which correlated with the expected mass of 7-methoxypsilocybin (FIG. 45G). The precursor compounds 7-methoxynorbaeocystin, 7-methoxybaeocystin were not detected, but were assumed to be present as intermediate products in the biosynthetic pathway.

Supplementation of 6-methylindole produced two new A280 product peaks on the HPLC chromatogram at 3.8 and 4.1 minutes. The peak at 3.8 minutes had a mass of 285, which correlated with the expected mass for 6-methylbaeocystin (FIG. 47F), while the peak at 4.1 minutes had a mass of 299, which correlated with the expected mass for 6-methylpsilocybin (FIG. 44F). Accumulation of 6-methylnorbaeocystin was not observed in the 6-methylpsilocybin production study, however when the psilocybin production strain, pSilo16, was replaced with the norbaeocystin production strain, pNor, 6-methylnorbaeocystin was observed as a terminal product (FIG. 50F).

Supplementation of 7-methylindole produced two new A280 product peaks on the HPLC chromatogram at 3.8 and 4.1 minutes. The small peak at 3.8 minutes had a mass of 285, which correlated with the expected mass for 7-methylbaeocystin (FIG. 48F), while the peak at 4.1 minutes had a mass of 299, which correlated with the expected mass of 7-methylpsilocybin (FIG. 45F). Accumulation of 7-methylnorbaeocystin was not observed in the 7-methylpsilocybin production study, however when the psilocybin production strain, pSilo16, was replaced with the norbaeocystin production strain, pNor, 7-methylnorbaeocystin was observed as a terminal product (FIG. 51F).

Structural insights on the psilocybin enzymes can be gained based on the substrate promiscuity observed across our indole library. Looking at the 2-position modifications, 2-chloroindole and 2-methylindole were converted to their corresponding tryptophan derivatives, with 2-methylindole processing completely. Interestingly, the smaller 2-fluoroindole failed to process through TrpB, suggesting the high electronegativity of the fluorine substitution, rather than size alone, may be responsible for the lack of activity.

Despite production of 2-chlorotryptophan and 2-methyltryptophan, neither tryptamine product was observed in the presence of PsiD. This suggests that PsiD shows limited capacity to accommodate side chain modifications at the 2-position. This observation was rare, as PsiD showed an ability to act on a broad range of tryptophan substrates, showing some level of conversion for all derivatives at the 4, 5, 6, and 7-position that successfully passed through TrpB, with the exception of 7-cyanotryptophan and 7-nitrotryptophan. This broad substrate promiscuity may lead to PsiD finding usefulness in other biosynthesis applications beyond those described above, such as in the biosynthesis of derivatives of N,N-dimethyltryptamine or through direct in vitro bioconversion applications.

Looking at the halogen series (F, Cl, Br, I) we can probe the sensitivity to side chain size and electronegativity at various substitution positions. For TrpB, the 4 and 5-position appear to be rather sensitive to the size of the side chain substitution with yield rapidly decreasing with side chain size increasing. The 6-position, however, showed somewhat an opposite tread with the highest yield being achieved for the iodo derivative. Interestingly, the 7-position showed near complete conversion of all 4 substrates, indicating high substrate flexibility at that position. Extending this analysis to PsiD, in the most optimized cases we see complete conversion of nearly all available tryptophan derivatives, indicating broad substrate flexibility as compared to that of TrpB. One interesting observation was that of the 5-bromo substitution, where no formation of 5-bromotryptophan was observed, however, upon promoter optimization of PsiD, 5-bromotryptamine was produced. This indicates that the downstream pathway optimization of PsiD expression was capable of pulling metabolite flux through TrpB, showing more substrate flexibility than observed from direct tests on TrpB in vivo.

Expanding the analysis to look at PsiH substrate flexibility, we find that 13 of 21 tryptamine products with an open 4-position were hydroxylated at the 4-position by PsiH. This trend shows a strong correlation with position of the substitution with 12 of the 13 successful cases having the side chain at with the 6- or 7-position, while 5 of the 8 unsuccessful cases were substituted at the 5-position. We hypothesize that the 5-position's proximity to the 4-position undergoing hydroxylation results in steric interactions limiting PsiH activity. It is also important to note that the only 5-position variant to be successful was the 5-fluoro, the smallest sidechain tested. This not only speaks to the substrate flexibility of PsiH, but due to the co-culture screening approach employed in this work, it also suggests that these tryptamine derivatives face minimal barriers to transport across the E. coli cell membrane.

Further extending the pathway to evaluate PsiK, we observe measurable conversion to the norbaeocystin derivative for all 13 sidechains which successfully passed through PsiH. The methyltransferase step, catalyzed by PsiM, did show a strict sensitivity to the presence of the 4-phosphoroxy group, with no observation of tryptamine or hydroxytryptamine methylation, which is consistent with previous studies (Adams et al., 2019; Fricke et al., 2017). Despite this limitation, PsiM showed no further restrictions, showing activity towards all 13 available phosphorylated tryptamine derivatives, resulting in the production of 13 non-natural psilocybin derivatives. Despite demonstrating activity, there was an accumulation of the N-methyl intermediates for 6-bromoindole, 6-chloroindole, 6-iodoindole, and, 6-methylindole and a corresponding reduction in the N,N-dimethyl products when compared to the corresponding substitutions at the 7-position. This reveals that substrates with 6-position substitutions are more hindered than their corresponding 7-position isomers when serving as a substrate for iterative methylations with PsiM, however, when compared to the natural substrate norbaeocystin (4-phosphoroxy only), both the 6 and 7 substituted derivatives showed significant restriction. This suggests that the additional space provided by the 7-position substitution has a lesser impact on PsiM activity, yet still shows a measurable reduction in activity due to the side chain presence.

An E. coli-based psilocybin production platform capable of de novo psilocybin production was developed. This system underwent initial genetic and process optimization which was necessary to leverage its enhanced biosynthesis capabilities for the production of a range of psilocybin derivatives and related intermediate products, as well as to inform the analysis of substrate flexibility in this biosynthesis platform. To that end, we tested a library of 45 substituted indoles in an E. coli-based psilocybin biosynthesis platform and found that 35 (78%) of them processed through TrpB to produce their respective substituted tryptophan products. The overall efficiency of processing for most of these substituted indoles was increased with the addition of serine. After performing transcriptional optimization on the PsiD expression vectors, 31 of 35 (89%) substituted indoles, which processed through TrpB, were decarboxylated by PsiD to produce their respective substituted tryptamine products. Thirteen (13) of these tryptamine products were processed completely by the remaining portion of the psilocybin pathway (PsiHKM), resulting in psilocybin derivative compounds. These exciting results highlight the power of this bacterial-based production platform and demonstrate the potential for production of various modified psilocybin derivatives using a bioprocessing approach. Further optimization is needed to produce higher yields of these products in order to purify and test them for bioactivity and efficacy in animal models of disease. While some of these target products are naturally occurring, many of them are non-natural in origin, and have not yet been evaluated for neurological activity, representing potential new drug products that may have therapeutic value for a range of conditions.

TABLE 1
List of Strains and Plasmids

| Number | Name | Description | Reference |
|---|---|---|---|
| S1 | Escherichia coli | F⁻, φ80d lacZΔM15, | Novagen |
| | DH5α | Δ(lacZYA-argF)U169, | |
| | | recA1, endA1, hsdR17(rk⁻, | |
| | | mk⁺), phoA, supE44λ⁻, | |
| | | thi⁻¹, gyrA96, relA1 | |
| S2 | Escherichia coli | F⁻ompT gal dcm rne131 lon | Invitrogen |
| | BL21 Star ™ | hsdS_{B} (r_{B}−m_{B}−) λ(DE3) | |
| | (DE3) | | |
| 1 | pETM6 | ColE1(pBR322), Amp^{R} | (Xu et al., |
| | | | 2012) |
| 2 | pETM6-mCherry | #1 containing the red | (Xu et al., |
| | | fluorescence reporter | 2012) |
| | | mCherry under the control | |
| | | of the consensus T7 | |
| | | promoter. | |
| 3 | pETM6-G6- | #2 containing the mutant | (Jones et |
| | mCherry | T7 promoter G6 | al., 2015) |
| 4 | pETM6-H9- | #2 containing the mutant | (Jones et |
| | mCherry | T7 promoter H9 | al., 2015) |
| 5 | pETM6-H10- | #2 containing the mutant | (Jones et |
| | mCherry | T7 promoter H10 | al., 2015) |
| 6 | pETM6-C4- | #2 containing the mutant | (Jones et |
| | mCherry | T7 promoter C4 | al., 2015) |
| 7 | pETM6-SDM2x | ColE1(pBR322), Amp^{R} | (Adams et |
| | | | al., 2019) |
| 8 | pETM6-SDM2x- | #7 with Psilocybe cubensis | This work |
| | PcCPR | CPR with N-term solubility | |
| | | tag | |
| 9 | pETM6-SDM2x- | #7 with Taxus cuspidata | This work |
| | TcCPR | CPR with N-term solubility | |
| | | tag | |
| 10 | pETM6-SDM2x- | #7 with Psilocybe cubensis | This work |
| | PcCPR_NoSol | CPR without N-term | |
| | | solubility tag | |
| 11 | pETM6-SDM2x- | #7 with Taxus cuspidata | This work |
| | TcCPR_NoSol | CPR without N-term | |
| | | solubility tag | |
| 12 | pETM6-SDM2x- | #7 with Psilocybe cubensis | This work |
| | PsiH (Piscu) | PsiH | |
| 13 | pETM6-SDM2x- | #7 with Psilocybe | This work |
| | PsiH (Psicy1) | cyanescens PsiH1 | |
| 14 | pETM6-SDM2x- | #7 with Psilocybe | This work |
| | PsiH (Psicy2) | cyanescens PsiH2 | |
| 15 | pETM6-SDM2x- | #7 with Gymnopilus dilepis | This work |
| | PsiH (Gymdi) | PsiH | |
| 16 | pETM6-SDM2x- | #7 with Panaeolus | This work |
| | PsiH (Pancy) | cyanescens PsiH | |
| 17 | pSilo16 | Optimized psilocybin | (Adams et |
| | | production pathway | al., 2019) |
| | | containing PsiDKM in | |
| | | operon format under the | |
| | | control of the H10 mutant | |
| | | T7 promoter. | |
| 18 | pNor (or Nor1) | Optimized norbaeocystin | (Adams et |
| | | production pathway | al., 2022) |
| | | containing PsiDK in operon | |
| | | format under the control of | |
| | | the C4 mutant T7 promoter. | |
| 19 | pCDM4-SDM2x | CloDF13, Str^{R} | (Adams et |
| | | | al., 2019) |
| 20 | pCDM4-SDM2x- | #19 Containing Psilocybe | This work |
| | PsiD | cubensis PsiD | |
| 21 | pACM4-SDM2x | P15A(pACYC184), Cm^{R} | (Adams et |
| | | | al., 2019) |
| 22 | pACM4-SDM2x- | #21 Containing Psilocybe | This work |
| | PsiD | cubensis PsiD | |
| 23 | pETM6-SDM2x- | #7 with Psilocybe cubensis | (Adams et |
| | PsiD | PsiD | al., 2019) |
| 24 | pETM6-G6-PsiD | #3 containing psiD with T7 | This work |
| | | mutant promoter G6 | |
| 25 | pETM6-H9-PsiD | #4 containing psiD with T7 | This work |
| | | mutant promoter H9 | |
| 26 | pETM6-H10- | #5 containing psiD with T7 | This work |
| | PsiD | mutant promoter H10 | |
| 27 | pETM6-C4-PsiD | #6 containing psiD with T7 | This work |
| | | mutant promoter C4 | |
| 28 | pXPA-fapO- | mCherry reporter under the | (Xu et al., |
| | mCherry | control of the constitutive | 2014) |
| | | GAP promoter. | |
| 29 | pXPA-PsiD | #28 Containing Psilocybe | This work |
| | | cubensis PsiD | |
| 30 | pXylA-mCherry | mCherry reporter under the | (Englaender |
| | | control of the constitutive | et al., 2017) |
| | | XylA promoter. | |
| 31 | pXylA-PsiD | #31 Containing Psilocybe | This work |
| | | cubensis PsiD | |
| 32 | pETM6-SDM2x- | JF01 operon configuration | This work |
| | PsiH-o.PcCPR | | |
| 33 | pETM6-SDM2x- | JF02 pseudooperon | This work |
| | PsiH-p.PcCPR | configuration | |
| 34 | pETM6-SDM2x- | JF03 monocistronic | This work |
| | PsiH-m.PcCPR | configuration | |
| 35 | pETM6-SDM2x- | JF04 operon configuration | This work |
| | PsiH-o.PcCPR_ | | |
| | NoSol | | |
| 36 | pETM6-SDM2x- | JF05 pseudooperon | This work |
| | PsiH-p.PcCPR_ | configuration | |
| | NoSol | | |
| 37 | pETM6-SDM2x- | JF06 monocistronic | This work |
| | PsiH-m.PcCPR_ | configuration | |
| | NoSol | | |
| 38 | pETM6-SDM2x- | JF07 operon configuration | This work |
| | PsiH-o.TcCPR | | |
| 39 | pETM6-SDM2x- | JF08 pseudooperon | This work |
| | PsiH-p.TcCPR | configuration | |
| 40 | pETM6-SDM2x- | JF09 monocistronic | This work |
| | PsiH-m.TcCPR | configuration | |
| 41 | pETM6-SDM2x- | JF10 operon configuration | This work |
| | PsiH-o.TcCPR_ | | |
| | NoSol | | |
| 42 | pETM6-SDM2x- | JF11 pseudooperon | This work |
| | PsiH-p.TcCPR_ | configuration | |
| | NoSol | | |
| 43 | pETM6-SDM2x- | JF12 monocistronic | This work |
| | PsiH-m.TcCPR_ | configuration | |
| | NoSol | | |
| 44 | pJF25 | Plasmid containing PsiH | (Hoefgen et |
| | | from Psilocybe cubensis | al., 2018) |
| 45 | pJF25_NdeI | #21 with internal NdeI site | This work |
| | Removed | removed | |

TABLE 2
Sequences

| SEQ | | |
|---|---|---|
| ID | | |
| NO: | Description | Sequence |
| 1 | Psilocybe | ATGGCACTATTGTTAGCTGTATTTAGGAGAGGAGGTTCTGCG |
| | cubensis | AGCTCTAGCAGCGATGTTTTTGTACTAGGTTTGGGTGTCGTC |
| | Cytochrome | CTGGCGGCGCTCTACATCTTTCGTGATCAACTGTTCGCCGCA |
| | P450 | AGCAAGCCGAAAGTGGCACCGGTTTCGACCACGAAACCGGC |
| | reductase | GAATGGTAGCGCGAACCCGCGCGACTTTATCGCTAAGATGA |
| | (CPR) | AACAAGGTAAGAAACGCATCGTGATTTTCTATGGCTCGCAA |
| | PcCPR with | ACCGGTACGGCTGAAGAATACGCAATTCGTCTAGCGAAAGA |
| | Solubility | GGCAAAGCAGAAATTCGGCTTGGCGAGCTTGGTTTGTGATCC |
| | Tag | GGAAGAATACGATTTCGAAAAACTGGACCAGCTGCCGGAGG |
| | | ACAGCATTGCGTTCTTTGTCGTTGCGACCTACGGCGAGGGTG |
| | | AGCCGACCGATAATGCAGTCCAACTGCTGCAAAACTTGCAA |
| | | GATGAAAGCTTTGAATTTTCCTCTGGTGAGCGCAAGTIGTCT |
| | | GGTCTGAAGTACGTGGTCTTTGGTCTGGGAAACAAAACGTA |
| | | CGAACATTACAATCTGATCGGTCGCACCGTGGACGCTCAGTT |
| | | GGCGAAGATGGGTGCAATCCGCATCGGCGAGCGTGGCGAGG |
| | | GTGACGACGACAAATCCATGGAAGAGGATTATCTCGAATGG |
| | | AAAGATGGCATGTGGGAAGCGTTCGCTACCGCAATGGGCGT |
| | | GGAGGAGGGCCAGGGTGGCGACAGCGCTGATTTCGTCGTTT |
| | | CCGAACTGGAATCACATCCACCGGAAAAAGTTTATCAGGGT |
| | | GAGTTCTCCGCTCGGGCATTGACCAAAACCAAGGGCATTCA |
| | | CGATGCGAAAAACCCGTTCGCGGCCCCGATCGCCGTGGCGC |
| | | GTGAACTTTTCCAGTCCGTCGTTGACCGTAATTGTGTGCATG |
| | | TGGAGTTCAACATCGAAGGCTCTGGTATCACCTACCAGCATG |
| | | GTGACCACGTGGGTCTGTGGCCGCTGAACCCGGACGTCGAA |
| | | GTGGAGCGCTTGCTTTGCGTTTTGGGCCTGGCGGAAAAACGT |
| | | GACGCAGTTATTAGCATCGAGTCGCTGGACCCGGCGCTTGCC |
| | | AAGGTGCCATTTCCGGTGCCGACTACGTACGGCGCGGTGCTG |
| | | CGCCACTACATTGATATCAGCGCGGTGGCTGGTCGTCAAATT |
| | | CTGGGCACCCTGAGCAAGTTCGCACCGACTCCGGAAGCTGA |
| | | GGCCTTCCTGCGAAATTTAAATACCAATAAAGAAGAGTACC |
| | | ATAACGTTGTGGCGAACGGTTGCCTGAAGCTCGGCGAGATC |
| | | CTGCAGATTGCGACCGGCAACGATATTACCGTTCCGCCTACG |
| | | ACCGCGAACACTACCAAATGGCCCATCCCGTTTGATATTATT |
| | | GTTAGCGCGATCCCGAGATTGCAACCGCGTTATTATTCCATC |
| | | TCCAGCAGCCCGAAAATCCACCCGAATACCATTCACGCGAC |
| | | GGTTGTCGTGCTGAAGTACGAGAACGTGCCAACTGAGCCTA |
| | | TCCCGCGTAAATGGGTTTACGGCGTTGGCAGCAATTTTTTGC |
| | | TGAACCTGAAGTATGCCGTGAACAAAGAGCCGGTTCCGTAC |
| | | ATCACCCAGAACGGCGAGCAGCGTGTGGGTGTACCGGAATA |
| | | CCTGATAGCGGGTCCGCGTGGTAGCTATAAGACCGAATCGTT |
| | | CTACAAGGCCCCAATCCACGTTCGTCGTTCTACCTTTCGTTTA |
| | | CCGACCAACCCGAAGTCCCCGGTGATCATGATTGGACCGGG |
| | | TACAGGTGTGGCTCCGTTCCGCGGTTTTGTTCAAGAACGCGT |
| | | GGCGCTGGCCCGTCGTAGCATCGAGAAGAACGGCCCAGATT |
| | | CCCTCGCAGATTGGGGTCGCATCAGCCTGTTTTACGGCTGCC |
| | | GTCGTAGTGATGAAGACTTCCTGTACAAGGACGAATGGCCG |
| | | CAGTATGAGGCGGAGCTGAAAGGCAAGTTTAAACTGCACTG |
| | | CGCATTCAGCCGTCAAAACTATAAGCCGGACGGCAGTAAAA |
| | | TTTATGTTCAGGACCTGATTTGGGAAGATCGCGAGCATATTG |
| | | CGGACGCCATCCTGAATGGTAAGGGTTATGTTTATATCTGCG |
| | | GTGAAGCCAAGAGCATGAGCAAGCAGGTTGAGGAGGTTTTG |
| | | GCTAAGATTCTGGGGGAGGCGAAGGGTGGCTCTGGTCCGGT |
| | | AGAGGGCGTTGCGGAGGTGAAATTGCTGAAAGAAAGATCTC |
| | | GTCTGATGCTGGACGTGTGGAGCTAA |
| 2 | Psilocybe | MALLLAVFRRGGSASSSSDVFVLGLGVVLAALYIFRDQLFAAS |
| | cubensis | KPKVAPVSTTKPANGSANPRDFIAKMKQGKKRIVIFYGSQTGT |
| | Cytochrome | AEEYAIRLAKEAKQKFGLASLVCDPEEYDFEKLDQLPEDSIAFF |
| | P450 | VVATYGEGEPTDNAVQLLQNLQDESFEFSSGERKLSGLKYVVF |
| | reductase | GLGNKTYEHYNLIGRTVDAQLAKMGAIRIGERGEGDDDKSME |
| | (CPR) | EDYLEWKDGMWEAFATAMGVEEGQGGDSADFVVSELESHPP |
| | PcCPR with | EKVYQGEFSARALTKTKGIHDAKNPFAAPIAVARELFQSVVDR |
| | Solubility | NCVHVEFNIEGSGITYQHGDHVGLWPLNPDVEVERLLCVLGLA |
| | Tag | EKRDAVISIESLDPALAKVPFPVPTTYGAVLRHYIDISAVAGRQI |
| | Amino Acid | LGTLSKFAPTPEAEAFLRNLNTNKEEYHNVVANGCLKLGEILQI |
| | Sequence | ATGNDITVPPTTANTTKWPIPFDIIVSAIPRLQPRYYSISSSPKIHP |
| | | NTIHATVVVLKYENVPTEPIPRKWVYGVGSNFLLNLKYAVNKE |
| | | PVPYITQNGEQRVGVPEYLIAGPRGSYKTESFYKAPIHVRRSTFR |
| | | LPTNPKSPVIMIGPGTGVAPFRGFVQERVALARRSIEKNGPDSLA |
| | | DWGRISLFYGCRRSDEDFLYKDEWPQYEAELKGKFKLHCAFSR |
| | | QNYKPDGSKIYVQDLIWEDREHIADAILNGKGYVYICGEAKSM |
| | | SKQVEEVLAKILGEAKGGSGPVEGVAEVKLLKERSRLMLDVW |
| | | S |
| 3 | Psilocybe | ATGGCGAGCTCTAGCAGCGATGTTTTTGTACTAGGTTTGGGT |
| | cubensis | GTCGTCCTGGCGGCGCTCTACATCTTTCGTGATCAACTGTTC |
| | Cytochrome | GCCGCAAGCAAGCCGAAAGTGGCACCGGTTTCGACCACGAA |
| | P450 | ACCGGCGAATGGTAGCGCGAACCCGCGCGACTTTATCGCTA |
| | reductase | AGATGAAACAAGGTAAGAAACGCATCGTGATTTTCTATGGC |
| | (CPR) | TCGCAAACCGGTACGGCTGAAGAATACGCAATTCGTCTAGC |
| | PcCPR | GAAAGAGGCAAAGCAGAAATTCGGCTTGGCGAGCTTGGTTT |
| | without | GTGATCCGGAAGAATACGATTTCGAAAAACTGGACCAGCTG |
| | Solubility | CCGGAGGACAGCATTGCGTTCTTTGTCGTTGCGACCTACGGC |
| | Tag | GAGGGTGAGCCGACCGATAATGCAGTCCAACTGCTGCAAAA |
| | | CTTGCAAGATGAAAGCTTTGAATTTTCCTCTGGTGAGCGCAA |
| | | GTTGTCTGGTCTGAAGTACGTGGTCTTTGGTCTGGGAAACAA |
| | | AACGTACGAACATTACAATCTGATCGGTCGCACCGTGGACG |
| | | CTCAGTTGGCGAAGATGGGTGCAATCCGCATCGGCGAGCGT |
| | | GGCGAGGGTGACGACGACAAATCCATGGAAGAGGATTATCT |
| | | CGAATGGAAAGATGGCATGTGGGAAGCGTTCGCTACCGCAA |
| | | TGGGCGTGGAGGAGGGCCAGGGTGGCGACAGCGCTGATTTC |
| | | GTCGTTTCCGAACTGGAATCACATCCACCGGAAAAAGTTTAT |
| | | CAGGGTGAGTTCTCCGCTCGGGCATTGACCAAAACCAAGGG |
| | | CATTCACGATGCGAAAAACCCGTTCGCGGCCCCGATCGCCGT |
| | | GGCGCGTGAACTTTTCCAGTCCGTCGTTGACCGTAATTGTGT |
| | | GCATGTGGAGTTCAACATCGAAGGCTCTGGTATCACCTACCA |
| | | GCATGGTGACCACGTGGGTCTGTGGCCGCTGAACCCGGACG |
| | | TCGAAGTGGAGCGCTTGCTTTGCGTTTTGGGCCTGGCGGAAA |
| | | AACGTGACGCAGTTATTAGCATCGAGTCGCTGGACCCGGCG |
| | | CTTGCCAAGGTGCCATTTCCGGTGCCGACTACGTACGGCGCG |
| | | GTGCTGCGCCACTACATTGATATCAGCGCGGTGGCTGGTCGT |
| | | CAAATTCTGGGCACCCTGAGCAAGTTCGCACCGACTCCGGA |
| | | AGCTGAGGCCTTCCTGCGAAATTTAAATACCAATAAAGAAG |
| | | AGTACCATAACGTIGTGGCGAACGGTTGCCTGAAGCTCGGC |
| | | GAGATCCTGCAGATTGCGACCGGCAACGATATTACCGTTCCG |
| | | CCTACGACCGCGAACACTACCAAATGGCCCATCCCGTTTGAT |
| | | ATTATTGTTAGCGCGATCCCGAGATTGCAACCGCGTTATTAT |
| | | TCCATCTCCAGCAGCCCGAAAATCCACCCGAATACCATTCAC |
| | | GCGACGGTTGTCGTGCTGAAGTACGAGAACGTGCCAACTGA |
| | | GCCTATCCCGCGTAAATGGGTTTACGGCGTTGGCAGCAATTT |
| | | TTTGCTGAACCTGAAGTATGCCGTGAACAAAGAGCCGGTTCC |
| | | GTACATCACCCAGAACGGCGAGCAGCGTGTGGGTGTACCGG |
| | | AATACCTGATAGCGGGTCCGCGTGGTAGCTATAAGACCGAA |
| | | TCGTTCTACAAGGCCCCAATCCACGTTCGTCGTTCTACCTTTC |
| | | GTTTACCGACCAACCCGAAGTCCCCGGTGATCATGATTGGAC |
| | | CGGGTACAGGTGTGGCTCCGTTCCGCGGTTTTGTTCAAGAAC |
| | | GCGTGGCGCTGGCCCGTCGTAGCATCGAGAAGAACGGCCCA |
| | | GATTCCCTCGCAGATTGGGGTCGCATCAGCCTGTTTTACGGC |
| | | TGCCGTCGTAGTGATGAAGACTTCCTGTACAAGGACGAATG |
| | | GCCGCAGTATGAGGCGGAGCTGAAAGGCAAGTTTAAACTGC |
| | | ACTGCGCATTCAGCCGTCAAAACTATAAGCCGGACGGCAGT |
| | | AAAATTTATGTTCAGGACCTGATTTGGGAAGATCGCGAGCAT |
| | | ATTGCGGACGCCATCCTGAATGGTAAGGGTTATGTTTATATC |
| | | TGCGGTGAAGCCAAGAGCATGAGCAAGCAGGTTGAGGAGGT |
| | | TTTGGCTAAGATTCTGGGGGAGGCGAAGGGTGGCTCTGGTC |
| | | CGGTAGAGGGCGTTGCGGAGGTGAAATTGCTGAAAGAAAGA |
| | | TCTCGTCTGATGCTGGACGTGTGGAGCTAA |
| 4 | Psilocybe | MASSSSDVFVLGLGVVLAALYIFRDQLFAASKPKVAPVSTTKP |
| | cubensis | ANGSANPRDFIAKMKQGKKRIVIFYGSQTGTAEEYAIRLAKEA |
| | Cytochrome | KQKFGLASLVCDPEEYDFEKLDQLPEDSIAFFVVATYGEGEPTD |
| | P450 | NAVQLLQNLQDESFEFSSGERKLSGLKYVVFGLGNKTYEHYNL |
| | reductase | IGRTVDAQLAKMGAIRIGERGEGDDDKSMEEDYLEWKDGMW |
| | (CPR) | EAFATAMGVEEGQGGDSADFVVSELESHPPEKVYQGEFSARAL |
| | PcCPR | TKTKGIHDAKNPFAAPIAVARELFQSVVDRNCVHVEFNIEGSGI |
| | without | TYQHGDHVGLWPLNPDVEVERLLCVLGLAEKRDAVISIESLDP |
| | Solubility | ALAKVPFPVPTTYGAVLRHYIDISAVAGRQILGTLSKFAPTPEAE |
| | Tag | AFLRNLNTNKEEYHNVVANGCLKLGEILQIATGNDITVPPTTAN |
| | Amino Acid | TTKWPIPFDIIVSAIPRLQPRYYSISSSPKIHPNTIHATVVVLKYEN |
| | Sequence | VPTEPIPRKWVYGVGSNFLLNLKYAVNKEPVPYITQNGEQRVG |
| | | VPEYLIAGPRGSYKTESFYKAPIHVRRSTFRLPTNPKSPVIMIGPG |
| | | TGVAPFRGFVQERVALARRSIEKNGPDSLADWGRISLFYGCRRS |
| | | DEDFLYKDEWPQYEAELKGKFKLHCAFSRQNYKPDGSKIYVQ |
| | | DLIWEDREHIADAILNGKGYVYICGEAKSMSKQVEEVLAKILGE |
| | | AKGGSGPVEGVAEVKLLKERSRLMLDVWS |
| 5 | Taxus | ATGGCACTATTGTTAGCTGTATTTAGGAGAGGAGGTTCTGAC |
| | cuspidata | ACGCAGAAACCGGCGGTGCGTCCGACCCCGCTGGTGAAGGA |
| | Cytochrome | AGAGGACGAAGAGGAGGAAGACGATAGCGCAAAGAAGAAG |
| | P450 | GTGACTATCTTTTTCGGCACCCAAACCGGCACTGCCGAAGGT |
| | reductase | TTTGCCAAGGCTCTCGCCGAGGAAGCGAAAGCGCGTTATGA |
| | (CPR) | AAAGGCAGTCTTTAAAGTTGTTGACCTGGACAACTATGCGGC |
| | TcCPR with | TGACGATGAGCAGTATGAGGAGAAATTGAAGAAGGAAAAG |
| | Solubility | TTAGCATTCTTTATGCTGGCTACCTACGGCGACGGCGAACCG |
| | Tag | ACGGATAATGCGGCGCGTTTCTATAAATGGTTCCTGGAAGGT |
| | | AAGGAGCGCGAGCCGTGGCTGAGCGACCTGACGTATGGTGT |
| | | GTTCGGTTTGGGGAACCGTCAGTATGAGCACTTTAATAAAGT |
| | | TGCGAAGGCGGTTGATGAGGTGTTGATTGAACAAGGTGCGA |
| | | AGAGATTGGTTCCGGTCGGTCTGGGCGATGACGACCAGTGT |
| | | ATTGAGGACGATTTTACCGCGTGGCGTGAGCAGGTTTGGCCG |
| | | GAGTTGGACCAGCTGCTGCGTGACGAGGATGATGAACCGAC |
| | | TTCTGCGACCCCTTATACCGCAGCTATCCCGGAATACCGTGT |
| | | CGAAATTTACGACTCCGTGGTTAGCGTGTACGAGGAGACCC |
| | | ATGCGCTTAAGCAGAACGGTCAGGCAGTTTATGATATTCACC |
| | | ATCCGTGTCGTTCTAACGTTGCGGTGCGTCGTGAGCTGCATA |
| | | CCCCGCTGTCCGATCGTTCTTGTATTCACCTGGAATTCGATAT |
| | | CAGCGACACCGGTCTGATCTACGAGACCGGCGACCACGTGG |
| | | GTGTCCACACGGAAAACAGCATTGAAACAGTAGAGGAGGCA |
| | | GCGAAATTGTTAGGCTACCAGCTGGATACGATTTTCAGCGTT |
| | | CATGGTGATAAAGAAGACGGCACCCCGCTGGGTGGCTCTAG |
| | | CCTGCCGCCGCCATTTCCGGGTCCGTGCACCCTGCGTACCGC |
| | | CTTGGCGCGTTACGCAGACTTGCTGAATCCGCCTCGTAAAGC |
| | | GGCGTTTCTGGCGCTGGCGGCGCATGCGTCAGACCCGGCAG |
| | | AGGCCGAACGTCTGAAATTTCTGAGTTCCCCGGCGGGTAAA |
| | | GATGAATATTCTCAATGGGTTACCGCGTCCCAACGTAGCCTG |
| | | CTTGAGATTATGGCCGAGTTTCCGAGCGCGAAGCCGCCGTTA |
| | | GGTGTGTTCTTTGCAGCGATCGCACCGCGCCTGCAACCGCGC |
| | | TACTATAGCATCTCCAGCTCTCCGCGTTTCGCTCCGTCCCGC |
| | | ATCCACGTTACCTGCGCCTTAGTCTACGGTCCAAGTCCGACC |
| | | GGTCGCATTCACAAGGGTGTGTGCAGCAACTGGATGAAAAA |
| | | CAGCCTGCCGAGCGAGGAAACGCATGATTGCAGCTGGGCAC |
| | | CAGTTTTCGTGCGCCAAAGCAACTTTAAGCTTCCGGCGGATT |
| | | CGACCACCCCCATCGTAATGGTCGGTCCAGGTACGGGCTTCG |
| | | CTCCTTTTAGAGGTTTTCTGCAAGAACGCGCAAAGCTGCAAG |
| | | AAGCTGGTGAAAAATTGGGTCCGGCGGTTCTGTTCTTCGGGT |
| | | GCCGTAATCGCCAGATGGATTACATTTACGAGGACGAACTG |
| | | AAGGGCTACGTCGAGAAAGGTATTCTGACCAATCTGATCGT |
| | | GGCCTTCAGCCGTGAAGGCGCGACCAAAGAGTATGTGCAGC |
| | | ATAAAATGTTGGAGAAGGCTTCGGACACGTGGAGCTTAATC |
| | | GCCCAGGGCGGCTACCTGTATGTTTGCGGTGATGCAAAAGG |
| | | CATGGCGCGCGATGTTCACCGTACCCTGCACACCATCGTTCA |
| | | GGAGCAAGAAAGCGTGGATTCTTCGAAAGCTGAGTTCTTGG |
| | | TGAAAAAGTTGCAAATGGATGGCCGCTACCTGCGTGACATC |
| | | TGGTAA |
| 6 | Taxus | MALLLAVFRRGGSDTQKPAVRPTPLVKEEDEEEEDDSAKKKV |
| | cuspidata | TIFFGTQTGTAEGFAKALAEEAKARYEKAVFKVVDLDNYAAD |
| | Cytochrome | DEQYEEKLKKEKLAFFMLATYGDGEPTDNAARFYKWFLEGKE |
| | P450 | REPWLSDLTYGVFGLGNRQYEHFNKVAKAVDEVLIEQGAKRL |
| | reductase | VPVGLGDDDQCIEDDFTAWREQVWPELDQLLRDEDDEPTSATP |
| | (CPR) | YTAAIPEYRVEIYDSVVSVYEETHALKQNGQAVYDIHHPCRSN |
| | TcCPR with | VAVRRELHTPLSDRSCIHLEFDISDTGLIYETGDHVGVHTENSIE |
| | Solubility | TVEEAAKLLGYQLDTIFSVHGDKEDGTPLGGSSLPPPFPGPCTL |
| | Tag | RTALARYADLLNPPRKAAFLALAAHASDPAEAERLKFLSSPAG |
| | Amino Acid | KDEYSQWVTASQRSLLEIMAEFPSAKPPLGVFFAAIAPRLQPRY |
| | Sequence | YSISSSPRFAPSRIHVTCALVYGPSPTGRIHKGVCSNWMKNSLPS |
| | | EETHDCSWAPVFVRQSNFKLPADSTTPIVMVGPGTGFAPFRGFL |
| | | QERAKLQEAGEKLGPAVLFFGCRNRQMDYIYEDELKGYVEKGI |
| | | LTNLIVAFSREGATKEYVQHKMLEKASDTWSLIAQGGYLYVCG |
| | | DAKGMARDVHRTLHTIVQEQESVDSSKAEFLVKKLQMDGRYL |
| | | RDIW |
| 7 | Taxus | ATGGACACGCAGAAACCGGCGGTGCGTCCGACCCCGCTGGT |
| | cuspidata | GAAGGAAGAGGACGAAGAGGAGGAAGACGATAGCGCAAAG |
| | Cytochrome | AAGAAGGTGACTATCTTTTTCGGCACCCAAACCGGCACTGCC |
| | P450 | GAAGGTTTTGCCAAGGCTCTCGCCGAGGAAGCGAAAGCGCG |
| | reductase | TTATGAAAAGGCAGTCTTTAAAGTTGTTGACCTGGACAACTA |
| | (CPR) | TGCGGCTGACGATGAGCAGTATGAGGAGAAATTGAAGAAGG |
| | TcCPR | AAAAGTTAGCATTCTTTATGCTGGCTACCTACGGCGACGGCG |
| | without | AACCGACGGATAATGCGGCGCGTTTCTATAAATGGTTCCTGG |
| | Solubility | AAGGTAAGGAGCGCGAGCCGTGGCTGAGCGACCTGACGTAT |
| | Tag | GGTGTGTTCGGTTTGGGGAACCGTCAGTATGAGCACTTTAAT |
| | | AAAGTTGCGAAGGCGGTTGATGAGGTGTTGATTGAACAAGG |
| | | TGCGAAGAGATTGGTTCCGGTCGGTCTGGGCGATGACGACC |
| | | AGTGTATTGAGGACGATTTTACCGCGTGGCGTGAGCAGGTTT |
| | | GGCCGGAGTTGGACCAGCTGCTGCGTGACGAGGATGATGAA |
| | | CCGACTTCTGCGACCCCTTATACCGCAGCTATCCCGGAATAC |
| | | CGTGTCGAAATTTACGACTCCGTGGTTAGCGTGTACGAGGAG |
| | | ACCCATGCGCTTAAGCAGAACGGTCAGGCAGTTTATGATATT |
| | | CACCATCCGTGTCGTTCTAACGTTGCGGTGCGTCGTGAGCTG |
| | | CATACCCCGCTGTCCGATCGTTCTTGTATTCACCTGGAATTC |
| | | GATATCAGCGACACCGGTCTGATCTACGAGACCGGCGACCA |
| | | CGTGGGTGTCCACACGGAAAACAGCATTGAAACAGTAGAGG |
| | | AGGCAGCGAAATTGTTAGGCTACCAGCTGGATACGATTTTCA |
| | | GCGTTCATGGTGATAAAGAAGACGGCACCCCGCTGGGTGGC |
| | | TCTAGCCTGCCGCCGCCATTTCCGGGTCCGTGCACCCTGCGT |
| | | ACCGCCTTGGCGCGTTACGCAGACTTGCTGAATCCGCCTCGT |
| | | AAAGCGGCGTTTCTGGCGCTGGCGGCGCATGCGTCAGACCC |
| | | GGCAGAGGCCGAACGTCTGAAATTTCTGAGTTCCCCGGCGG |
| | | GTAAAGATGAATATTCTCAATGGGTTACCGCGTCCCAACGTA |
| | | GCCTGCTTGAGATTATGGCCGAGTTTCCGAGCGCGAAGCCGC |
| | | CGTTAGGTGTGTTCTTTGCAGCGATCGCACCGCGCCTGCAAC |
| | | CGCGCTACTATAGCATCTCCAGCTCTCCGCGTTTCGCTCCGT |
| | | CCCGCATCCACGTTACCTGCGCCTTAGTCTACGGTCCAAGTC |
| | | CGACCGGTCGCATTCACAAGGGTGTGTGCAGCAACTGGATG |
| | | AAAAACAGCCTGCCGAGCGAGGAAACGCATGATTGCAGCTG |
| | | GGCACCAGTTTTCGTGCGCCAAAGCAACTTTAAGCTTCCGGC |
| | | GGATTCGACCACCCCCATCGTAATGGTCGGTCCAGGTACGG |
| | | GCTTCGCTCCTTTTAGAGGTTTTCTGCAAGAACGCGCAAAGC |
| | | TGCAAGAAGCTGGTGAAAAATTGGGTCCGGCGGTTCTGTTCT |
| | | TCGGGTGCCGTAATCGCCAGATGGATTACATTTACGAGGAC |
| | | GAACTGAAGGGCTACGTCGAGAAAGGTATTCTGACCAATCT |
| | | GATCGTGGCCTTCAGCCGTGAAGGCGCGACCAAAGAGTATG |
| | | TGCAGCATAAAATGTTGGAGAAGGCTTCGGACACGTGGAGC |
| | | TTAATCGCCCAGGGCGGCTACCTGTATGTTTGCGGTGATGCA |
| | | AAAGGCATGGCGCGCGATGTTCACCGTACCCTGCACACCAT |
| | | CGTTCAGGAGCAAGAAAGCGTGGATTCTTCGAAAGCTGAGT |
| | | TCTTGGTGAAAAAGTTGCAAATGGATGGCCGCTACCTGCGTG |
| | | ACATCTGGTAA |
| 8 | Taxus | MDTQKPAVRPTPLVKEEDEEEEDDSAKKKVTIFFGTQTGTAEG |
| | cuspidata | FAKALAEEAKARYEKAVFKVVDLDNYAADDEQYEEKLKKEK |
| | Cytochrome | LAFFMLATYGDGEPTDNAARFYKWFLEGKEREPWLSDLTYGV |
| | P450 | FGLGNRQYEHFNKVAKAVDEVLIEQGAKRLVPVGLGDDDQCI |
| | reductase | EDDFTAWREQVWPELDQLLRDEDDEPTSATPYTAAIPEYRVEI |
| | (CPR) | YDSVVSVYEETHALKQNGQAVYDIHHPCRSNVAVRRELHTPLS |
| | TcCPR | DRSCIHLEFDISDTGLIYETGDHVGVHTENSIETVEEAAKLLGYQ |
| | without | LDTIFSVHGDKEDGTPLGGSSLPPPFPGPCTLRTALARYADLLNP |
| | Solubility | PRKAAFLALAAHASDPAEAERLKFLSSPAGKDEYSQWVTASQR |
| | Tag | SLLEIMAEFPSAKPPLGVFFAAIAPRLQPRYYSISSSPRFAPSRIHV |
| | Amino Acid | TCALVYGPSPTGRIHKGVCSNWMKNSLPSEETHDCSWAPVFVR |
| | Sequence | QSNFKLPADSTTPIVMVGPGTGFAPFRGFLQERAKLQEAGEKLG |
| | | PAVLFFGCRNRQMDYIYEDELKGYVEKGILTNLIVAFSREGATK |
| | | EYVQHKMLEKASDTWSLIAQGGYLYVCGDAKGMARDVHRTL |
| | | HTIVQEQESVDSSKAEFLVKKLQMDGRYLRDIW |
| 9 | PsiH | ATGATCGCTGTACTATTCTCCTTCGTCATTGCAGGATGCATA |
| | (MF000993) | TACTACATCGTTTCTCGTAGAGTGAGGCGGTCGCGCTTGCCA |
| | | CCAGGGCCGCCTGGCATTCCTATTCCCTTCATTGGGAACATG |
| | | TTTGATATGCCTGAAGAATCTCCATGGTTAACATTTCTACAA |
| | | TGGGGACGGGATTACAACACCGATATTCTCTACGTGGATGCT |
| | | GGAGGGACAGAAATGGTTATTCTTAACACGTTGGAGACCAT |
| | | TACCGATCTATTAGAAAAGCGAGGGTCCATTTATTCTGGCCG |
| | | ACTTGAGAGTACAATGGTCAACGAACTTATGGGGTGGGAGT |
| | | TTGACTTAGGGTTCATCACATACGGCGACAGGTGGCGCGAA |
| | | GAAAGGCGCATGTTCGCCAAGGAGTTCAGTGAGAAGGGCAT |
| | | CAAGCAATTTCGCCATGCTCAAGTGAAAGCTGCCCATCAGCT |
| | | TGTCCAACAGCTTACCAAAACGCCAGACCGCTGGGCACAAC |
| | | ATATTCGCCATCAGATAGCGGCAATGTCACTGGATATTGGTT |
| | | ATGGAATTGATCTTGCAGAAGACGACCCTTGGCTGGAAGCG |
| | | ACCCATTTGGCTAATGAAGGCCTCGCCATAGCATCAGTGCCG |
| | | GGCAAATTTTGGGTCGATTCGTTCCCTTCTCTAAAATACCTTC |
| | | CTGCTTGGTTCCCAGGTGCTGTCTTCAAGCGCAAAGCGAAGG |
| | | TCTGGCGAGAAGCCGCCGACCACATGGTTGACATGCCTTATG |
| | | AAACTATGAGGAAATTAGCACCTCAAGGATTGACTCGTCCG |
| | | TCGTATGCTTCAGCTCGTCTGCAAGCCATGGATCTCAACGGT |
| | | GACCTTGAGCATCAAGAACACGTAATCAAGAACACAGCCGC |
| | | AGAGGTTAATGTCGGTGGAGGCGATACTACTGTCTCTGCTAT |
| | | GTCTGCGTTCATCTTGGCCATGGTGAAGTACCCTGAGGTCCA |
| | | GCGAAAGGTTCAAGCGGAGCTTGATGCTCTGACCAATAACG |
| | | GCCAAATTCCTGACTATGACGAAGAAGATGACTCCTTGCCAT |
| | | ACCTCACCGCATGTATCAAGGAGCTTTTCCGGTGGAATCAAA |
| | | TCGCACCCCTCGCTATACCGCACAAATTAATGAAGGACGAC |
| | | GTGTACCGCGGGTATCTGATTCCCAAGAACACTCTAGTCTTC |
| | | GCAAACACCTGGGCAGTATTAAACGATCCAGAAGTCTATCC |
| | | AGATCCCTCTGTGTTCCGCCCAGAAAGATATCTTGGTCCTGA |
| | | CGGGAAGCCTGATAACACTGTACGCGACCCACGTAAAGCGG |
| | | CATTTGGCTATGGACGACGAAATTGTCCCGGAATTCATCTAG |
| | | CGCAGTCGACGGTTTGGATTGCAGGGGCAACCCTCTTATCAG |
| | | CGTTCAATATCGAGCGACCTGTCGATCAGAATGGGAAGCCC |
| | | ATTGACATACCGGCTGATTTTACTACAGGATTCTTCAGACAC |
| | | CCAGTGCCTTTCCAGTGCAGGTTTGTTCCTCGAACAGAGCAA |
| | | GTCTCACAGTCGGTATCCGGACCCTGA |
| 10 | PsiH | MIAVLFSFVIAGCIYYIVSRRVRRSRLPPGPPGIPIPFIGNMFDMP |
| | (Genbank | EESPWLTFLQWGRDYNTDILYVDAGGTEMVILNTLETITDLLEK |
| | MF000993) | RGSIYSGRLESTMVNELMGWEFDLGFITYGDRWREERRMFAKE |
| | Amino Acid | FSEKGIKQFRHAQVKAAHQLVQQLTKTPDRWAQHIRHQIAAM |
| | Sequence | SLDIGYGIDLAEDDPWLEATHLANEGLAIASVPGKFWVDSFPSL |
| | | KYLPAWFPGAVFKRKAKVWREAADHMVDMPYETMRKLAPQ |
| | | GLTRPSYASARLQAMDLNGDLEHQEHVIKNTAAEVNVGGGDT |
| | | TVSAMSAFILAMVKYPEVQRKVQAELDALTNNGQIPDYDEED |
| | | DSLPYLTACIKELFRWNQIAPLAIPHKLMKDDVYRGYLIPKNTL |
| | | VFANTWAVLNDPEVYPDPSVFRPERYLGPDGKPDNTVRDPRK |
| | | AAFGYGRRNCPGIHLAQSTVWIAGATLLSAFNIERPVDQNGKPI |
| | | DIPADFTTGFFRHPVPFQCRFVPRTEQVSQSVSGP |
| 11 | PsiD | ATGCAGGTGATACCCGCGTGCAACTCGGCAGCAATAAGATC |
| | (Genbank | ACTATGTCCTACTCCCGAGTCTTTTAGAAACATGGGATGGCT |
| | KY984101.1) | CTCTGTCAGCGATGCGGTCTACAGCGAGTTCATAGGAGAGTT |
| | | GGCTACCCGCGCTTCCAATCGAAATTACTCCAACGAGTTCGG |
| | | CCTCATGCAACCTATCCAGGAATTCAAGGCTTTCATTGAAAG |
| | | CGACCCGGTGGTGCACCAAGAATTTATTGACATGTTCGAGG |
| | | GCATTCAGGACTCTCCAAGGAATTATCAGGAACTATGTAATA |
| | | TGTTCAACGATATCTTTCGCAAAGCTCCCGTCTACGGAGACC |
| | | TTGGCCCTCCCGTTTATATGATTATGGCCAAATTAATGAACA |
| | | CCCGAGCGGGCTTCTCTGCATTCACGAGACAAAGGTTGAAC |
| | | CTTCACTTCAAAAAACTTTTCGATACCTGGGGATTGTTCCTG |
| | | TCTTCGAAAGATTCTCGAAATGTTCTTGTGGCCGACCAGTTC |
| | | GACGACAGACATTGCGGCTGGTTGAACGAGCGGGCCTTGTC |
| | | TGCTATGGTTAAACATTACAATGGACGCGCATTTGATGAAGT |
| | | CTTCCTCTGCGATAAAAATGCCCCATACTACGGCTTCAACTC |
| | | TTACGACGACTTCTTTAATCGCAGATTTCGAAACCGAGATAT |
| | | CGACCGACCTGTAGTCGGTGGAGTTAACAACACCACCCTCAT |
| | | TTCTGCTGCTTGCGAATCACTTTCCTACAACGTCTCTTATGAC |
| | | GTCCAGTCTCTCGACACTTTAGTTTTCAAAGGAGAGACTTAT |
| | | TCGCTTAAGCATTTGCTGAATAATGACCCTTTCACCCCACAA |
| | | TTCGAGCATGGGAGTATTCTACAAGGATTCTTGAACGTCACC |
| | | GCTTACCACCGATGGCACGCACCCGTCAATGGGACAATCGT |
| | | CAAAATCATCAACGTTCCAGGTACCTACTTTGCGCAAGCCCC |
| | | GAGCACGATTGGCGACCCTATCCCGGATAACGATTACGACC |
| | | CACCTCCTTACCTTAAGTCTCTTGTCTACTTCTCTAATATTGC |
| | | CGCAAGGCAAATTATGTTTATTGAAGCCGACAACAAGGAAA |
| | | TTGGCCTCATTTTCCTTGTGTTCATCGGCATGACCGAAATCTC |
| | | GACATGTGAAGCCACGGTGTCCGAAGGTCAACACGTCAATC |
| | | GTGGCGATGACTTGGGAATGTTCCATTTCGGTGGTTCTTCGT |
| | | TCGCGCTTGGTCTGAGGAAGGATTGCAGGGCAGAGATCGTT |
| | | GAAAAGTTCACCGAACCCGGAACAGTGATCAGAATCAACGA |
| | | AGTCGTCGCTGCTCTAAAGGCTTAG |
| 12 | PsiK | ATGGCGTTCGATCTCAAGACTGAAGACGGCCTCATCACATAT |
| | (Genbank | CTCACTAAACATCTTTCTTTGGACGTCGACACGAGCGGAGTG |
| | KY984099.1) | AAGCGCCTTAGCGGAGGCTTTGTCAATGTAACCTGGCGCATT |
| | | AAGCTCAATGCTCCTTATCAAGGTCATACGAGCATCATCCTG |
| | | AAGCATGCTCAGCCGCACATGTCTACGGATGAGGATTTTAA |
| | | GATAGGTGTAGAACGTTCGGTTTACGAATACCAGGCTATCA |
| | | AGCTCATGATGGCCAATCGGGAGGTTCTGGGAGGCGTGGAT |
| | | GGCATAGTTTCTGTGCCAGAAGGCCTGAACTACGACTTAGA |
| | | GAATAATGCATTGATCATGCAAGATGTCGGGAAGATGAAGA |
| | | CCCTTTTAGATTATGTCACCGCCAAACCGCCACTTGCGACGG |
| | | ATATAGCCCGCCTTGTTGGGACAGAAATTGGGGGGTTCGTTG |
| | | CCAGACTCCATAACATAGGCCGCGAGAGGCGAGACGATCCT |
| | | GAGTTCAAATTCTTCTCTGGAAATATTGTCGGAAGGACGACT |
| | | TCAGACCAGCTGTATCAAACCATCATACCCAACGCAGCGAA |
| | | ATATGGCGTCGATGACCCCTTGCTGCCTACTGTGGTTAAGGA |
| | | CCTTGTGGACGATGTCATGCACAGCGAAGAGACCCTTGTCAT |
| | | GGCGGACCTGTGGAGTGGAAATATTCTTCTCCAGTTGGAGG |
| | | AGGGAAACCCATCGAAGCTGCAGAAGATATATATCCTGGAT |
| | | TGGGAACTTTGCAAGTACGGCCCAGCGTCGTTGGACCTGGG |
| | | CTATTTCTTGGGTGACTGCTATTTGATATCCCGCTTTCAAGAC |
| | | GAGCAGGTCGGTACGACGATGCGGCAAGCCTACTTGCAAAG |
| | | CTATGCGCGTACGAGCAAGCATTCGATCAACTACGCCAAAG |
| | | TCACTGCAGGTATTGCTGCTCATATTGTGATGTGGACCGACT |
| | | TTATGCAGTGGGGGAGCGAGGAAGAAAGGATAAATTTTGTG |
| | | AAAAAGGGGGTAGCTGCCTTTCACGACGCCAGGGGCAACAA |
| | | CGACAATGGGGAAATTACGTCTACCTTACTGAAGGAATCAT |
| | | CCACTGCGTAA |
| 13 | PsiM | ATGCATATCAGAAATCCTTACCGTACACCAATTGACTATCAA |
| | (Genbank | GCACTTTCAGAGGCCTTCCCTCCCCTCAAGCCATTTGTGTCT |
| | KY984100.1) | GTCAATGCAGATGGTACCAGTTCTGTTGACCTCACTATCCCA |
| | | GAAGCCCAGAGGGCGTTCACGGCCGCTCTTCTTCATCGTGAC |
| | | TTCGGGCTCACCATGACCATACCAGAAGACCGTCTGTGCCCA |
| | | ACAGTCCCCAATAGGTTGAACTACGTTCTGTGGATTGAAGAT |
| | | ATTTTCAACTACACGAACAAAACCCTCGGCCTGTCGGATGAC |
| | | CGTCCTATTAAAGGCGTTGATATTGGTACAGGAGCCTCCGCA |
| | | ATTTATCCTATGCTTGCCTGTGCTCGGTTCAAGGCATGGTCT |
| | | ATGGTTGGAACAGAGGTCGAGAGGAAGTGCATTGACACGGC |
| | | CCGCCTCAATGTCGTCGCGAACAATCTCCAAGACCGTCTCTC |
| | | GATATTAGAGACATCCATTGATGGTCCTATTCTCGTCCCCAT |
| | | TTTCGAGGCGACTGAAGAATACGAATACGAGTTTACTATGTG |
| | | TAACCCTCCATTCTACGACGGTGCTGCCGATATGCAGACTTC |
| | | GGATGCTGCCAAAGGATTTGGATTTGGCGTGGGCGCTCCCCA |
| | | TTCTGGAACAGTCATCGAAATGTCGACTGAGGGAGGTGAAT |
| | | CGGCTTTCGTCGCTCAGATGGTCCGTGAGAGCTTGAAGCTTC |
| | | GAACACGATGCAGATGGTACACGAGTAACTTGGGAAAGCTG |
| | | AAATCCTTGAAAGAAATAGTGGGGCTGCTGAAAGAACTTGA |
| | | GATAAGCAACTATGCCATTAACGAATACGTTCAGGGGTCCA |
| | | CACGTCGTTATGCCGTTGCGTGGTCTTTCACTGATATTCAACT |
| | | GCCTGAGGAGCTTTCTCGTCCCTCTAACCCCGAGCTCAGCTC |
| | | TCTTTTCTAG |
| 14 | PsiD | MQVIPACNSAAIRSLCPTPESFRNMGWLSVSDAVYSEFIGELAT |
| | amino acid | RASNRNYSNEFGLMQPIQEFKAFIESDPVVHQEFIDMFEGIQDSP |
| | sequence | RNYQELCNMFNDIFRKAPVYGDLGPPVYMIMAKLMNTRAGFS |
| | | AFTRQRLNLHFKKLFDTWGLFLSSKDSRNVLVADQFDDRHCG |
| | | WLNERALSAMVKHYNGRAFDEVFLCDKNAPYYGFNSYDDFFN |
| | | RRFRNRDIDRPVVGGVNNTTLISAACESLSYNVSYDVQSLDTLV |
| | | FKGETYSLKHLLNNDPFTPQFEHGSILQGFLNVTAYHRWHAPV |
| | | NGTIVKIINVPGTYFAQAPSTIGDPIPDNDYDPPPYLKSLVYFSNI |
| | | AARQIMFIEADNKEIGLIFLVFIGMTEISTCEATVSEGQHVNRGD |
| | | DLGMFHFGGSSFALGLRKDCRAEIVEKFTEPGTVIRINEVVAAL |
| | | KA |
| 15 | PsiK | MAFDLKTEDGLITYLTKHLSLDVDTSGVKRLSGGFVNVTWRIK |
| | amino acid | LNAPYQGHTSIILKHAQPHMSTDEDFKIGVERSVYEYQAIKLM |
| | sequence | MANREVLGGVDGIVSVPEGLNYDLENNALIMQDVGKMKTLLD |
| | | YVTAKPPLATDIARLVGTEIGGFVARLHNIGRERRDDPEFKFFS |
| | | GNIVGRTTSDQLYQTIIPNAAKYGVDDPLLPTVVKDLVDDVMH |
| | | SEETLVMADLWSGNILLQLEEGNPSKLQKIYILDWELCKYGPAS |
| | | LDLGYFLGDCYLISRFQDEQVGTTMRQAYLQSYARTSKHSINY |
| | | AKVTAGIAAHIVMWTDFMQWGSEEERINFVKKGVAAFHDARG |
| | | NNDNGEITSTLLKESSTA |
| 16 | PsiM | MHIRNPYRTPIDYQALSEAFPPLKPFVSVNADGTSSVDLTIPEAQ |
| | amino acid | RAFTAALLHRDFGLTMTIPEDRLCPTVPNRLNYVLWIEDIFNYT |
| | sequence | NKTLGLSDDRPIKGVDIGTGASAIYPMLACARFKAWSMVGTEV |
| | | ERKCIDTARLNVVANNLQDRLSILETSIDGPILVPIFEATEEYEYE |
| | | FTMCNPPFYDGAADMQTSDAAKGFGFGVGAPHSGTVIEMSTE |
| | | GGESAFVAQMVRESLKLRTRCRWYTSNLGKLKSLKEIVGLLKE |
| | | LEISNYAINEYVQGSTRRYAVAWSFTDIQLPEELSRPSNPELSSL |
| | | F |
| 17 | H10 mutant | TAATACGACTCACTACGGAAGAA |
| | T7 promoter | |
| 18 | G6 mutant T7 | TAATACGACTCACTATTTCGGAA |
| | promoter | |
| 19 | H9 mutant T7 | TAATACGACTCACTAATACTGAA |
| | promoter | |
| 20 | C4 mutant T7 | TAATACGACTCACTATCAAGGAA |
| | promoter | |
| 21 | consensus T7 | TAATACGACTCACTATAGGGGAA |
| | promoter | |
| 22 | Lac promoter | TTTACACTTTATGCTTCCGGCTCGTATGTTG |
| 23 | Lac UV5 | TTTACACTTTATGCTTCCGGCTCGTATAATG |
| | promoter | |
| 24 | tac promoter | TTGACAATTAATCATCGGCTCGTATAATG |
| 25 | trc promoter | TTGACAATTAATCATCCGGCTCGTATAATG |
| 26 | GAP | GCGTAATGCTTAGGCACAGGATTGATTTGTCGCAATGATTGA |
| | promoter | CACGATTCCGCTTGACGCTGCGTAAGGTTTTTGTAATTTTAC |
| | | AGGCAACCTTTTATTCA |
| 27 | xylA | TTGAAATAAACATTTATTTTGTATATGATGAGATAAAGTTAG |
| | promoter | TTTATTGGATAAACAAACTAACTCAATTAAGATAGTTGATGG |
| | | ATAAACTT |
| 28 | pPsilo16 | TAATACGACTCACTACGGAAGAATTGTGAGCGGATAACAAT |
| | vector | TCCCCTCTAGAAATAATTTTGTTTAACTTTAAGAAGGAGATA |
| | (Also | TACATATGGCTAGCATGACTGGTGGACAGCAAATGGGTCGC |
| | referred | GGATCCATGCAGGTGATACCCGCGTGCAACTCGGCAGCAAT |
| | to as | AAGATCACTATGTCCTACTCCCGAGTCTTTTAGAAACATGGG |
| | “pSilo16”; | ATGGCTCTCTGTCAGCGATGCGGTCTACAGCGAGTTCATAGG |
| | See | AGAGTTGGCTACCCGCGCTTCCAATCGAAATTACTCCAACGA |
| | WO 2021/ | GTTCGGCCTCATGCAACCTATCCAGGAATTCAAGGCTTTCAT |
| | 086513) | TGAAAGCGACCCGGTGGTGCACCAAGAATTTATTGACATGTT |
| | | CGAGGGCATTCAGGACTCTCCAAGGAATTATCAGGAACTAT |
| | | GTAATATGTTCAACGATATCTTTCGCAAAGCTCCCGTCTACG |
| | | GAGACCTTGGCCCTCCCGTTTATATGATTATGGCCAAATTAA |
| | | TGAACACCCGAGCGGGCTTCTCTGCATTCACGAGACAAAGG |
| | | TTGAACCTTCACTTCAAAAAACTTTTCGATACCTGGGGATTG |
| | | TTCCTGTCTTCGAAAGATTCTCGAAATGTTCTTGTGGCCGAC |
| | | CAGTTCGACGACAGACATTGCGGCTGGTTGAACGAGCGGGC |
| | | CTTGTCTGCTATGGTTAAACATTACAATGGACGCGCATTTGA |
| | | TGAAGTCTTCCTCTGCGATAAAAATGCCCCATACTACGGCTT |
| | | CAACTCTTACGACGACTTCTTTAATCGCAGATTTCGAAACCG |
| | | AGATATCGACCGACCTGTAGTCGGTGGAGTTAACAACACCA |
| | | CCCTCATTTCTGCTGCTTGCGAATCACTTTCCTACAACGTCTC |
| | | TTATGACGTCCAGTCTCTCGACACTTTAGTTTTCAAAGGAGA |
| | | GACTTATTCGCTTAAGCATTTGCTGAATAATGACCCTTTCAC |
| | | CCCACAATTCGAGCATGGGAGTATTCTACAAGGATTCTTGAA |
| | | CGTCACCGCTTACCACCGATGGCACGCACCCGTCAATGGGA |
| | | CAATCGTCAAAATCATCAACGTTCCAGGTACCTACTTTGCGC |
| | | AAGCCCCGAGCACGATTGGCGACCCTATCCCGGATAACGAT |
| | | TACGACCCACCTCCTTACCTTAAGTCTCTTGTCTACTTCTCTA |
| | | ATATTGCCGCAAGGCAAATTATGTTTATTGAAGCCGACAACA |
| | | AGGAAATTGGCCTCATTTTCCTTGTGTTCATCGGCATGACCG |
| | | AAATCTCGACATGTGAAGCCACGGTGTCCGAAGGTCAACAC |
| | | GTCAATCGTGGCGATGACTTGGGAATGTTCCATTTCGGTGGT |
| | | TCTTCGTTCGCGCTTGGTCTGAGGAAGGATTGCAGGGCAGAG |
| | | ATCGTTGAAAAGTTCACCGAACCCGGAACAGTGATCAGAAT |
| | | CAACGAAGTCGTCGCTGCTCTAAAGGCTTAGAAGCTTGCGG |
| | | CCGCACTCGAGTCTGGTAAAGAAACCGCTGCTGCGAAATTT |
| | | GAACGCCAGCACATGGACTCGTCTACTAGAAATAATTTTGTT |
| | | TAACTTTAAGAAGGAGATATACATATGGCTAGCATGACTGG |
| | | TGGACAGCAAATGGGTCGCGGATCCATGGCGTTCGATCTCA |
| | | AGACTGAAGACGGCCTCATCACATATCTCACTAAACATCTTT |
| | | CTTTGGACGTCGACACGAGCGGAGTGAAGCGCCTTAGCGGA |
| | | GGCTTTGTCAATGTAACCTGGCGCATTAAGCTCAATGCTCCT |
| | | TATCAAGGTCATACGAGCATCATCCTGAAGCATGCTCAGCCG |
| | | CACATGTCTACGGATGAGGATTTTAAGATAGGTGTAGAACG |
| | | TTCGGTTTACGAATACCAGGCTATCAAGCTCATGATGGCCAA |
| | | TCGGGAGGTTCTGGGAGGCGTGGATGGCATAGTTTCTGTGCC |
| | | AGAAGGCCTGAACTACGACTTAGAGAATAATGCATTGATCA |
| | | TGCAAGATGTCGGGAAGATGAAGACCCTTTTAGATTATGTCA |
| | | CCGCCAAACCGCCACTTGCGACGGATATAGCCCGCCTTGTTG |
| | | GGACAGAAATTGGGGGGTTCGTTGCCAGACTCCATAACATA |
| | | GGCCGCGAGAGGCGAGACGATCCTGAGTTCAAATTCTTCTCT |
| | | GGAAATATTGTCGGAAGGACGACTTCAGACCAGCTGTATCA |
| | | AACCATCATACCCAACGCAGCGAAATATGGCGTCGATGACC |
| | | CCTTGCTGCCTACTGTGGTTAAGGACCTTGTGGACGATGTCA |
| | | TGCACAGCGAAGAGACCCTTGTCATGGCGGACCTGTGGAGT |
| | | GGAAATATTCTTCTCCAGTTGGAGGAGGGAAACCCATCGAA |
| | | GCTGCAGAAGATATATATCCTGGATTGGGAACTTTGCAAGTA |
| | | CGGCCCAGCGTCGTTGGACCTGGGCTATTTCTTGGGTGACTG |
| | | CTATTTGATATCCCGCTTTCAAGACGAGCAGGTCGGTACGAC |
| | | GATGCGGCAAGCCTACTTGCAAAGCTATGCGCGTACGAGCA |
| | | AGCATTCGATCAACTACGCCAAAGTCACTGCAGGTATTGCTG |
| | | CTCATATTGTGATGTGGACCGACTTTATGCAGTGGGGGAGCG |
| | | AGGAAGAAAGGATAAATTTTGTGAAAAAGGGGGTAGCTGCC |
| | | TTTCACGACGCCAGGGGCAACAACGACAATGGGGAAATTAC |
| | | GTCTACCTTACTGAAGGAATCATCCACTGCGTAAAAGCTTGC |
| | | GGCCGCACTCGAGTCTGGTAAAGAAACCGCTGCTGCGAAAT |
| | | TTGAACGCCAGCACATGGACTCGTCTACTAGAAATAATTTTG |
| | | TTTAACTTTAAGAAGGAGATATACATATGGCTAGCATGACTG |
| | | GTGGACAGCAAATGGGTCGCGGATCCATGCATATCAGAAAT |
| | | CCTTACCGTACACCAATTGACTATCAAGCACTTTCAGAGGCC |
| | | TTCCCTCCCCTCAAGCCATTTGTGTCTGTCAATGCAGATGGT |
| | | ACCAGTTCTGTTGACCTCACTATCCCAGAAGCCCAGAGGGCG |
| | | TTCACGGCCGCTCTTCTTCATCGTGACTTCGGGCTCACCATG |
| | | ACCATACCAGAAGACCGTCTGTGCCCAACAGTCCCCAATAG |
| | | GTTGAACTACGTTCTGTGGATTGAAGATATTTTCAACTACAC |
| | | GAACAAAACCCTCGGCCTGTCGGATGACCGTCCTATTAAAG |
| | | GCGTTGATATTGGTACAGGAGCCTCCGCAATTTATCCTATGC |
| | | TTGCCTGTGCTCGGTTCAAGGCATGGTCTATGGTTGGAACAG |
| | | AGGTCGAGAGGAAGTGCATTGACACGGCCCGCCTCAATGTC |
| | | GTCGCGAACAATCTCCAAGACCGTCTCTCGATATTAGAGACA |
| | | TCCATTGATGGTCCTATTCTCGTCCCCATTTTCGAGGCGACTG |
| | | AAGAATACGAATACGAGTTTACTATGTGTAACCCTCCATTCT |
| | | ACGACGGTGCTGCCGATATGCAGACTTCGGATGCTGCCAAA |
| | | GGATTTGGATTTGGCGTGGGCGCTCCCCATTCTGGAACAGTC |
| | | ATCGAAATGTCGACTGAGGGAGGTGAATCGGCTTTCGTCGCT |
| | | CAGATGGTCCGTGAGAGCTTGAAGCTTCGAACACGATGCAG |
| | | ATGGTACACGAGTAACTTGGGAAAGCTGAAATCCTTGAAAG |
| | | AAATAGTGGGGCTGCTGAAAGAACTTGAGATAAGCAACTAT |
| | | GCCATTAACGAATACGTTCAGGGGTCCACACGTCGTTATGCC |
| | | GTTGCGTGGTCTTTCACTGATATTCAACTGCCTGAGGAGCTT |
| | | TCTCGTCCCTCTAACCCCGAGCTCAGCTCTCTTTTCTAGCTCG |
| | | AGTCTGGTAAAGAAACCGCTGCTGCGAAATTTGAACGCCAG |
| | | CACATGGACTCGTCTACTAGTCGCAGCTTAATTAACCTAAAC |
| | | TGCTGCCACCGCTGAGCAATAACTAGCATAACCCCTTGGGGC |
| | | CTCTAAACGGGTCTTGAGGGGTTTTTTGCTAGCGAAAGGAGG |
| | | AGTCGACTATATCCGGATTGGCGAATGGGACGCGCCCTGTA |
| | | GCGGCGCATTAAGCGCGGCGGGTGTGGTGGTTACGCGCAGC |
| | | GTGACCGCTACACTTGCCAGCGCCCTAGCGCCCGCTCCTTTC |
| | | GCTTTCTTCCCTTCCTTTCTCGCCACGTTCGCCGGCTTTCCCC |
| | | GTCAAGCTCTAAATCGGGGGCTCCCTTTAGGGTTCCGATTTA |
| | | GTGCTTTACGGCACCTCGACCCCAAAAAACTTGATTAGGGTG |
| | | ATGGTTCACGTAGTGGGCCATCGCCCTGATAGACGGTTTTTC |
| | | GCCCTTTGACGTTGGAGTCCACGTTCTTTAATAGTGGACTCT |
| | | TGTTCCAAACTGGAACAACACTCAACCCTATCTCGGTCTATT |
| | | CTTTTGATTTATAAGGGATTTTGCCGATTTCGGCCTATTGGTT |
| | | AAAAAATGAGCTGATTTAACAAAAATTTAACGCGAATTTTA |
| | | ACAAAATATTAACGTTTACAATTTCTGGCGGCACGATGGCAT |
| | | GAGATTATCAAAAAGGATCTTCACCTAGATCCTTTTAAATTA |
| | | AAAATGAAGTTTTAAATCAATCTAAAGTATATATGAGTAAA |
| | | CTTGGTCTGACAGTTACCAATGCTTAATCAGTGAGGCACCTA |
| | | TCTCAGCGATCTGTCTATTTCGTTCATCCATAGTTGCCTGACT |
| | | CCCCGTCGTGTAGATAACTACGATACGGGAGGGCTTACCATC |
| | | TGGCCCCAGTGCTGCAATGATACCGCGAGACCCACGCTCAC |
| | | CGGCTCCAGATTTATCAGCAATAAACCAGCCAGCCGGAAGG |
| | | GCCGAGCGCAGAAGTGGTCCTGCAACTTTATCCGCCTCCATC |
| | | CAGTCTATTAATTGTTGCCGGGAAGCTAGAGTAAGTAGTTCG |
| | | CCAGTTAATAGTTTGCGCAACGTTGTTGCCATTGCTACAGGC |
| | | ATCGTGGTGTCACGCTCGTCGTTTGGTATGGCTTCATTCAGC |
| | | TCCGGTTCCCAACGATCAAGGCGAGTTACATGATCCCCCATG |
| | | TTGTGCAAAAAAGCGGTTAGCTCCTTCGGTCCTCCGATCGTT |
| | | GTCAGAAGTAAGTTGGCCGCAGTGTTATCACTCATGGTTATG |
| | | GCAGCACTGCATAATTCTCTTACTGTCATGCCATCCGTAAGA |
| | | TGCTTTTCTGTGACTGGTGAGTACTCAACCAAGTCATTCTGA |
| | | GAATAGTGTATGCGGCGACCGAGTTGCTCTTGCCCGGCGTCA |
| | | ATACGGGATAATACCGCGCCACATAGCAGAACTTTAAAAGT |
| | | GCTCATCATTGGAAAACGTTCTTCGGGGCGAAAACTCTCAAG |
| | | GATCTTACCGCTGTTGAGATCCAGTTCGATGTAACCCACTCG |
| | | TGCACCCAACTGATCTTCAGCATCTTTTACTTTCACCAGCGTT |
| | | TCTGGGTGAGCAAAAACAGGAAGGCAAAATGCCGCAAAAA |
| | | AGGGAATAAGGGCGACACGGAAATGTTGAATACTCATACTC |
| | | TTCCTTTTTCAATCATGATTGAAGCATTTATCAGGGTTATTGT |
| | | CTCATGAGCGGATACATATTTGAATGTATTTAGAAAAATAAA |
| | | CAAATAGGTCATGACCAAAATCCCTTAACGTGAGTTTTCGTT |
| | | CCACTGAGCGTCAGACCCCGTAGAAAAGATCAAAGGATCTT |
| | | CTTGAGATCCTTTTTTTCTGCGCGTAATCTGCTGCTTGCAAAC |
| | | AAAAAAACCACCGCTACCAGCGGTGGTTTGTTTGCCGGATC |
| | | TAGGCCACCACTTCAAGAACTCTGTAGCACCGCCTACATACC |
| | | TCGCTCTGCTAATCCTGTTACCAGTGGCTGCTGCCAGTGGCG |
| | | ATAAGTCGTGTCTTACCGGGTTGGACTCAAGACGATAGTTAC |
| | | CGGATAAGGCGCAGCGGTCGGGCTGAACGGGGGGTTCGTGC |
| | | ACACAGCCCAGCTTGGAGCGAACGACCTACACCGAACTGAG |
| | | ATACCTACAGCGTGAGCTATGAGAAAGCGCCACGCTTCCCG |
| | | AAGGGAGAAAGGCGGACAGGTATCCGGTAAGCGGCAGGGT |
| | | CGGAACAGGAGAGCGCACGAGGGAGCTTCCAGGGGGAAAC |
| | | GCCTGGTATCTTTATAGTCCTGTCGGGTTTCGCCACCTCTGAC |
| | | TTGAGCGTCGATTTTTGTGATGCTCGTCAGGGGGGCGGAGCC |
| | | TATGGAAAAACGCCAGCAACGCGGCCTTTTTACGGTTCCTGG |
| | | CCTTTTGCTGGCCTTTTGCTCACATGTTCTTTCCTGCGTTATC |
| | | CCCTGATTCTGTGGATAACCGTATTACCGCCTTTGAGTGAGC |
| | | TGATACCGCTCGCCGCAGCCGAACGACCGAGCGCAGCGAGT |
| | | CAGTGAGCGAGGAAGCGGAAGAGCGCCTGATGCGGTATTTT |
| | | CTCCTTACGCATCTGTGCGGTATTTCACACCGCATATATGGT |
| | | GCACTCTCAGTACAATCTGCTCTGATGCCGCATAGTTAAGCC |
| | | AGTATACACTCCGCTATCGCTACGTGACTGGGTCATGGCTGC |
| | | GCCCCGACACCCGCCAACACCCGCTGACGCGCCCTGACGGG |
| | | CTTGTCTGCTCCCGGCATCCGCTTACAGACAAGCTGTGACCG |
| | | TCTCCGGGAGCTGCATGTGTCAGAGGTTTTCACCGTCATCAC |
| | | CGAAACGCGCGAGGCAGCTGCGGTAAAGCTCATCAGCGTGG |
| | | TCGTGAAGCGATTCACAGATGTCTGCCTGTTCATCCGCGTCC |
| | | AGCTCGTTGAGTTTCTCCAGAAGCGTTAATGTCTGGCTTCTG |
| | | ATAAAGCGGGCCATGTTAAGGGCGGTTTTTTCCTGTTTGGTC |
| | | ACTGATGCCTCCGTGTAAGGGGGATTTCTGTTCATGGGGGTA |
| | | ATGATACCGATGAAACGAGAGAGGATGCTCACGATACGGGT |
| | | TACTGATGATGAACATGCCCGGTTACTGGAACGTTGTGAGG |
| | | GTAAACAACTGGCGGTATGGATGCGGCGGGACCAGAGAAAA |
| | | ATCACTCAGGGTCAATGCCAGCGCTTCGTTAATACAGATGTA |
| | | GGTGTTCCACAGGGTAGCCAGCAGCATCCTGCGATGCAGAT |
| | | CCGGAACATAATGGTGCAGGGCGCTGACTTCCGCGTTTCCAG |
| | | ACTTTACGAAACACGGAAACCGAAGACCATTCATGTTGTTGC |
| | | TCAGGTCGCAGACGTTTTGCAGCAGCAGTCGCTTCACGTTCG |
| | | CTCGCGTATCGGTGATTCATTCTGCTAACCAGTAAGGCAACC |
| | | CCGCCAGCCTAGCCGGGTCCTCAACGACAGGAGCACGATCA |
| | | TGCTAGTCATGCCCCGCGCCCACCGGAAGGAGCTGACTGGG |
| | | TTGAAGGCTCTCAAGGGCATCGGTCGAGATCCCGGTGCCTA |
| | | ATGAGTGAGCTAACTTACATTAATTGCGTTGCGCTCACTGCC |
| | | CGCTTTCCAGTCGGGAAACCTGTCGTGCCAGCTGCATTAATG |
| | | AATCGGCCAACGCGCGGGGAGAGGCGGTTTGCGTATTGGGC |
| | | GCCAGGGTGGTTTTTCTTTTCACCAGTGAGACGGGCAACAGC |
| | | TGATTGCCCTTCACCGCCTGGCCCTGAGAGAGTTGCAGCAAG |
| | | CGGTCCACGCTGGTTTGCCCCAGCAGGCGAAAATCCTGTTTG |
| | | ATGGTGGTTAACGGCGGGATATAACATGAGCTGTCTTCGGTA |
| | | TCGTCGTATCCCACTACCGAGATGTCCGCACCAACGCGCAGC |
| | | CCGGACTCGGTAATGGCGCGCATTGCGCCCAGCGCCATCTG |
| | | ATCGTTGGCAACCAGCATCGCAGTGGGAACGATGCCCTCATT |
| | | CAGCATTTGCATGGTTTGTTGAAAACCGGACATGGCACTCCA |
| | | GTCGCCTTCCCGTTCCGCTATCGGCTGAATTTGATTGCGAGT |
| | | GAGATATTTATGCCAGCCAGCCAGACGCAGACGCGCCGAGA |
| | | CAGAACTTAATGGGCCCGCTAACAGCGCGATTTGCTGGTGA |
| | | CCCAATGCGACCAGATGCTCCACGCCCAGTCGCGTACCGTCT |
| | | TCATGGGAGAAAATAATACTGTTGATGGGTGTCTGGTCAGA |
| | | GACATCAAGAAATAACGCCGGAACATTAGTGCAGGCAGCTT |
| | | CCACAGCAATGGCATCCTGGTCATCCAGCGGATAGTTAATG |
| | | ATCAGCCCACTGACGCGTTGCGCGAGAAGATTGTGCACCGC |
| | | CGCTTTACAGGCTTCGACGCCGCTTCGTTCTACCATCGACAC |
| | | CACCACGCTGGCACCCAGTTGATCGGCGCGAGATTTAATCGC |
| | | CGCGACAATTTGCGACGGCGCGTGCAGGGCCAGACTGGAGG |
| | | TGGCAACGCCAATCAGCAACGACTGTTTGCCCGCCAGTTGTT |
| | | GTGCCACGCGGTTGGGAATGTAATTCAGCTCCGCCATCGCCG |
| | | CTTCCACTTTTTCCCGCGTTTTCGCAGAAACGTGGCTGGCCT |
| | | GGTTCACCACGCGGGAAACGGTCTGATAAGAGACACCGGCA |
| | | TACTCTGCGACATCGTATAACGTTACTGGTTTCACATTCACC |
| | | ACCCTGAATTGACTCTCTTCCGGGCGCTATCATGCCATACCG |
| | | CGAAAGGTTTTGCGCCATTCGATGGTGTCCGGGATCTCGACG |
| | | CTCTCCCTTATGCGACTCCTGCATTAGGAAGCAGCCCAGTAG |
| | | TAGGTTGAGGCCGTTGAGCACCGCCGCCGCAAGGAATGGTG |
| | | CATGCAAGGAGATGGCGCCCAACAGTCCCCCGGCCACGGGG |
| | | CCTGCCACCATACCCACGCCGAAACAAGCGCTCATGAGCCC |
| | | GAAGTGGCGAGCCCGATCTTCCCCATCGGTGATGTCGGCGAT |
| | | ATAGGCGCCAGCAACCGCACCTGTGGCGCCGGTGATGCCGG |
| | | CCACGATGCGTCCGGCGTAGCCTAGGATCGAGATCGATCTC |
| | | GATCCCGCGAAAT |

TABLE 3
List of Primers Used in this Study

| No | Name | Sequence |
|---|---|---|
| 1 | TcPcCPR_FWD_1 | GGTCCATATGGCACTATTGTTAGCTG (SEQ ID NO: 29) |
| 2 | FWD_Pc_nosolubility | CCGCGGCCATATGGCGAGCTCTAGCAGCGATG (SEQ |
| | | ID NO: 30) |
| 3 | FWD_Tc_nosolubility | CCGCGGCATATGGACACGCAGAAACCGGCG (SEQ ID |
| | | NO: 31) |
| 4 | PcCPR_Seq_1 | CCTCTGGTGAGCGCAAGTTGTC (SEQ ID NO: 32) |
| 5 | PcCPR_Seq_2 | GAAGTGGAGCGCTTGCTTTGC (SEQ ID NO: 33) |
| 6 | PcCPR_Seq_3 | GCTGAACCTGAAGTATGCCGTGAAC (SEQ ID NO: 34) |
| 7 | TcCPR_Seq_1 | GTGTTGATTGAACAAGGTGCGAAGAG (SEQ ID |
| | | NO: 35) |
| 8 | TcCPR_Seq 2 | GATAAAGAAGACGGCACCCCGCTG (SEQ ID NO: 36) |
| 9 | PcCPR_REV_1 | CGCTTTCGCTCGAGTTAGCTC (SEQ ID NO: 37) |
| 10 | TcCPR_REV_1 | CGCTTTCGCTCGAGTTACCAG (SEQ ID NO: 38) |
| 11 | PsiH_FWD_NdeI | GCGGCGCATATGATCGCTGTACTATTCTCCTTCGTC |
| | | (SEQ ID NO: 39) |
| 12 | PsiH_REV_XhoI | GCCGCGCTCGAGTCAGGGTCCGGATACCGACTG |
| | | (SEQ ID NO: 40) |
| 13 | SDM_PsiH_FWD | GAAGGTCTGGCGAGAAGCCGCCGACCACATGGTTGA |
| | | CATGCCTTATGAAACTATG (SEQ ID NO: 41) |
| 14 | SDM_PsiH_REV | CATAGTTTCATAAGGCATGTCAACCATGTGGTCGGC |
| | | GGCTTCTCGCCAGACCTTC (SEQ ID NO: 42) |
| 15 | Psicu_PsiH_FullLength_FWD | GCCCGCCATATGGCGCTGCTGCTGG (SEQ ID NO: 43) |
| 16 | Psicu_PsiH_HalfTrunc_FWD | GCCCGCCATATGGCTCTGTTATTAGCAGTTTTTTACT |
| | | ACATCGTTTCTCGTAGAGTGAGGC (SEQ ID NO: 44) |
| 17 | Psicu_PsiH_KKT_FWD | GCCCGCCATATGGCTAAGAAAACGAGCTCTAAAGGG |
| | | AAGTTGCCACCAGGGCCGCC (SEQ ID NO: 45) |
| 18 | Psicu_PsiH_MA_FWD | GCCCGCCATATGGCGCGTAGAGTGAGGCGGTCGC |
| | | (SEQ ID NO: 46) |
| 19 | Psicu_PsiH_XhoI_REV | CGCTCGGCTCGAGTCAGGGTCCGGATACC (SEQ ID |
| | | NO: 47) |
| 20 | Psicy1_PsiH_FullLength_FWD | GCCCGCCATATGGCTCTGTTATTAGCAGTTTTTATTG |
| | | TGC (SEQ ID NO: 48) |
| 21 | Psicy1_PsiH_HalfTrunc_FWD | GCCCGCCATATGGCTCTGTTATTAGCAGTTTTCTACT |
| | | ATGCGAACGCGCGC (SEQ ID NO: 49) |
| 22 | Psicy1_PsiH_KKT_FWD | GCCCGCCATATGGCTAAGAAAACGAGCTCTAAAGGG |
| | | AAGCTTCCGCCTGGCCCG (SEQ ID NO: 50) |
| 23 | Psicy1_PsiH_MA_FWD | GCCCGCCATATGGCCCGCAGAGTGCGTCGCAG (SEQ |
| | | ID NO: 51) |
| 24 | Psicy1_PsiH_XhoI_REV | TCTCGGCTCGAGTCAGCCGCTCACG (SEQ ID NO: 52) |
| 25 | Gymnopilus_PsiH_FullLength_FWD | GCCCGCCATATGGCTCTGTTATTAGCAGTTTTTCAAG |
| | | G (SEQ ID NO: 53) |
| 26 | Gymnopilus_PsiH_HalfTrunc_FWD | GCCCGCCATATGGCTCTGTTATTAGCAGTTTTTTGCG |
| | | TGTACTACGCACACTCTC (SEQ ID NO: 54) |
| 27 | Gymnopilus_PsiH_KKT_FWD | GCCCGCCATATGGCTAAGAAAACGAGCTCTAAAGGG |
| | | AAGCTGCCTCCTGGACCCCCG (SEQ ID NO: 55) |
| 28 | Gymnopilus_PsiH_MA_FWD | GCCCGCCATATGGCCCGTCGTGCGCGGC (SEQ ID |
| | | NO: 56) |
| 29 | Gymnopilus_PsiH_XhoI_REV | GCGTCGCTCGAGTCACACATCTTGCACCG (SEQ ID |
| | | NO: 57) |
| 30 | Gymnopilus_PsiH_MA_FWD_Long | GCCCGCCATATGGCCCGTCGTGCGCGGCGTGC (SEQ |
| | | ID NO: 58) |
| 31 | Panaeolus_PsiH_FullLength_FWD | GCCCGCCATATGGCTCTGTTATTAGCAGTTTTTATTA |
| | | ACCTG (SEQ ID NO: 59) |
| 32 | Panaeolus_PsiH_HalfTrunc_FWD | GCCCGCCATATGGCTCTGTTATTAGCAGTTTTTTATT |
| | | ATATTGTGAGCCGCCGCATTC (SEQ ID NO: 60) |
| 33 | Panaeolus_PsiH_KKT_FWD | GCCCGCCATATGGCTAAGAAAACGAGCTCTAAAGGG |
| | | AAGCTGCCACCTGGCCCACCG (SEQ ID NO: 61) |
| 34 | Panaeolus_PsiH_MA_FWD | GCCCGCCATATGGCTCGCCGCATTCGCCGCAG (SEQ |
| | | ID NO: 62) |
| 35 | Panaeolus_PsiH_XhoI_REV | GCTCGCGCTCGAGTCACAGGCCGCTCAG (SEQ ID |
| | | NO: 63) |
| 36 | Psicy2_PsiH_FullLength_FWD | GCCCGCCATATGGCTCTGTTATTAGCAGTTTTTGCG |
| | | (SEQ ID NO: 64) |
| 37 | Psicy2_PsiH_HalfTrunc_FWD | GCCCGCCATATGGCTCTGTTATTAGCAGTTTTTGGCT |
| | | GCATTTATTATATTAACGCACGTCG (SEQ ID NO: 65) |
| 38 | Psicy2_PsiH_KKT_FWD | GCCCGCCATATGGCTAAGAAAACGAGCTCTAAAGGG |
| | | AAGCTGCCTCCTGGCCCTCCTG (SEQ ID NO: 66) |
| 39 | Psicy2_PsiH_MA_FWD | GCCCGCCATATGGCCAACGCACGTCGCATTAAACG |
| | | (SEQ ID NO: 67) |
| 40 | Psicy 2_PsiH_XhoI_REV | GCTCCGCTCGAGTCAGCGAAAAAAGCCG (SEQ ID |
| | | NO: 68) |
| 41 | pcCPR_FullLength_FWD | GCCCGCCATATGGCACTATTGTTAGCTGTATTTAGGA |
| | | GAG (SEQ ID NO: 69) |
| 42 | pcCPR_KKT_FWD | GCCCGCCATATGGCTAAGAAAACGAGCTCTAAAGGG |
| | | AAGCTCCCACCAGGACCATCGACCACGAAACCGGC |
| | | G (SEQ ID NO: 70) |
| 43 | pcCPR_MA_FWD | GCCCGCCATATGGCACGTGATCAACTGTTCGCCG |
| | | (SEQ ID NO: 71) |
| 44 | pcCPR_MALLLAVF_FWD | GCCCGCCATATGGCTCTGTTATTAGCAGTTTTCCGTG |
| | | ATCAACTGTTCGCCG (SEQ ID NO: 72) |
| 45 | pcCPR_XhoI_Rev | TCTCGGCTCGAGTTAGCTCCACACGTCC (SEQ ID |
| | | NO: 73) |

### BIBLIOGRAPHY

Adams, A. M., Anas, N. A., Sen, A. K., Hinegardner-Hendricks, J. D., O'Dell, P. J., Gibbons, W. J., Flower, J. E., McMurray, M. S., Jones, J. A., 2022. Development of an E. coli-based norbaeocystin production platform and evaluation of behavioral effects in rats. Metab Eng Commun 14.
Adams, A. M., Kaplan, N. A., Wei, Z., Brinton, J. D., Monnier, C. S., Enacopol, A. L., Ramelot, T. A., Jones, J. A., 2019. In vivo production of psilocybin in E. coli. Metab Eng 56, 111-119.
Biggs, B. W., Lim, C. G., Sagliani, K., Shankar, S., Stephanopoulos, G., De Mey, M., Ajikumar, P. K., 2016. Overcoming heterologous protein interdependency to optimize P450-mediated Taxol precursor synthesis in Escherichia coli. Proc Natl Acad Sci USA 113, 3209-14.
Blei, F., Baldeweg, F., Fricke, J., Hoffmeister, D., 2018. Biocatalytic Production of Psilocybin and Derivatives in Tryptophan Synthase-Enhanced Reactions. Chemistry—A European Journal 24, 10028-10031.
Boville, C. E., Romney, D. K., Almhjell, P. J., Sieben, M., Arnold, F. H., 2018a. Improved Synthesis of 4-Cyanotryptophan and Other Tryptophan Analogues in Aqueous Solvent Using Variants of TrpB from Thermotoga maritima. Journal of Organic Chemistry 83, 7447-7452.
Boville, C. E., Scheele, R. A., Koch, P., Brinkmann-Chen, S., Buller, A. R., Arnold, F. H., 2018b. Engineered Biosynthesis of R-Alkyl Tryptophan Analogues. Angewandte Chemie International Edition 57, 14764-14768.
Buller, A. R., Brinkmann-Chen, S., Romney, D. K., Herger, M., Murciano-Calles, J., Arnold, F. H., 2015. Directed evolution of the tryptophan synthase β-subunit for stand-alone function recapitulates allosteric activation. Proc Natl Acad Sci USA 112, 14599-14604.
Cao, D., Yu, J., Wang, H., Luo, Z., Liu, X., He, L., Qi, J., Fan, L., Tang, L., Chen, Z., Li, J., Cheng, J., Wang, S., 2022. Structure-based discovery of nonhallucinogenic psychedelic analogs. Science (1979) 375, 403-411.
Carhart-Harris, R. L., Bolstridge, M., Rucker, J., Day, C. M. J., Erritzoe, D., Kaelen, M., Bloomfield, M., Rickard, J. A., Forbes, B., Feilding, A., Taylor, D., Pilling, S., Curran, V. H., Nutt, D. J., 2016. Psilocybin with psychological support for treatment-resistant depression: an open-label feasibility study. Lancet Psychiatry 3, 619-627.
Carhart-Harris, R. L., Roseman, L., Bolstridge, M., Demetriou, L., Pannekoek, J. N., Wall, M. B., Tanner, M., Kaelen, M., McGonigle, J., Murphy, K., Leech, R., Curran, H. V., Nutt, D. J., 2017. Psilocybin for treatment-resistant depression: fMRI-measured brain mechanisms. Sci Rep 7, 13187.
Cash, M. T., Miles, E. W., Phillips, R. S., 2004. The reaction of indole with the aminoacrylate intermediate of Salmonella typhimurium tryptophan synthase: observation of a primary kinetic isotope effect with 3-[2H]indole. Arch Biochem Biophys 432, 233-243.
Crowley, P. B., Kyne, C., Monteith, W. B., 2012. Simple and inexpensive incorporation of 19F-Tryptophan for protein NMR spectroscopy. Chemical Communications 48, 10681-10683.
Englaender, J. A., Jones, J. A., Cress, B. F., Kuhlman, T. E., Linhardt, R. J., Koffas, M. A. G., 2017. Effect of Genomic Integration Location on Heterologous Protein Expression and Metabolic Engineering in E. coli. ACS Synth Biol 6.
Francis, D., Winn, M., Latham, J., Greaney, M. F., Micklefield, J., 2017. An Engineered Tryptophan Synthase Opens New Enzymatic Pathways to β-Methyltryptophan and Derivatives. ChemBioChem 18, 382-386.
Fricke, J., Blei, F., Hoffmeister, D., 2017. Enzymatic Synthesis of Psilocybin. Angewandte Chemie International Edition 56, 12352-12355.
Fricke, J., Sherwood, A., Kargbo, R., Orry, A., Blei, F., Naschberger, A., Rupp, B., Hoffmeister, D., 2019. Enzymatic Route toward 6-Methylated Baeocystin and Psilocybin. ChemBioChem 20, 2824-2829.
Fricke, J., Sherwood, A. M., Halberstadt, A. L., Kargbo, R. B., Hoffmeister, D., 2021. Chemoenzymatic Synthesis of 5-Methylpsilocybin: A Tryptamine with Potential Psychedelic Activity. J Nat Prod 84, 1403-1408.
Garcia-Romeu, A., R. Griffiths, R., W. Johnson, M., 2014. Psilocybin-Occasioned Mystical Experiences in the Treatment of Tobacco Addiction. Curr Drug Abuse Rev 7, 157-164.
Gibbons, W. J., McKinney, M. G., O'Dell, P. J., Bollinger, B. A., Jones, J. A., 2021. Homebrewed psilocybin: can new routes for pharmaceutical psilocybin production enable recreational use? Bioengineered 12, 8863-8871.
Glennon, R. A., Schubert, E., Jacyno, J. M., Rosecrans, J. A., 1980. Studies on Several 7-Substituted N, N-Dimethyltryptamines. J Med Chem 23, 1222-1226.
Gluff, J. A., Teolis, M. G., Moore, A. A., Kelly, D. R., 2017. Post-traumatic Stress Disorder (PTSD): A Webliography. J Consum Health Internet 21, 389-401.
Goss, R. J. M., Newill, P. L. A., 2006. A convenient enzymatic synthesis of L-halotryptophans. Chemical Communications 4924-4925.
HALL, A. N., LEA, D. J., RYDON, H. N., 1962. The behaviour of the Bz-methylindoles as substrates and inhibitors for Neurospora crassa tryptophan synthase. Biochemical Journal 84, 12-16.
Hausjell, J., Halbwirth, H., Spadiut, O., 2018. Recombinant production of eukaryotic cytochrome P450s in microbial cell factories. Biosci Rep 38, 20171290.
He, W., Fu, L., Li, G., Andrew Jones, J., Linhardt, R. J., Koffas, M., 2015. Production of chondroitin in metabolically engineered E. coli. Metab Eng 27, 92-100.
Held, W. A., Smith, O. H., 1970. Mechanism of 3-Methylanthranilic Acid Derepression of the Tryptophan Operon in Escherichia coli. J Bacteriol 101, 209-217.
Herger, M., van Roye, P., Romney, D. K., Brinkmann-Chen, S., Buller, A. R., Arnold, F. H., 2016. Synthesis of 3-Branched Tryptophan Analogues Using an Engineered Subunit of Tryptophan Synthase. J Am Chem Soc 138, 8388-8391.
Hoefgen, S., Lin, J., Fricke, J., Stroe, M. C., Mattern, D. J., Kufs, J. E., Hortschansky, P., Brakhage, A. A., Hoffmeister, D., Valiante, V., 2018. Facile assembly and fluorescence-based screening method for heterologous expression of biosynthetic pathways in fungi. Metab Eng 48, 44-51.
Hoffarth, E. R., Kong, S., He, H. Y., Ryan, K. S., 2021. An engineered biosynthetic-synthetic platform for production of halogenated indolmycin antibiotics. Chem Sci 12, 8817-8821.
Johnson, M. W., Griffiths, R. R., 2017. Potential Therapeutic Effects of Psilocybin. Neurotherapeutics 14, 734-740.
Jones, J. A., Vemacchio, V. R., Lachance, D. M., Lebovich, M., Fu, L., Shirke, A. N., Schultz, V. L., Cress, B., Linhardt, R. J., Koffas, M. A. G., 2015. ePathOptimize: A Combinatorial Approach for Transcriptional Balancing of Metabolic Pathways. Sci Rep 5, 11301.
Klein, A. K., Chatha, M., Laskowski, L. J., Anderson, E. I., Brandt, S. D., Chapman, S. J., Mccorvy, J. D., Halberstadt, A. L., 2021. Investigation of the Structure-Activity Relationships of Psilocybin Analogues. ACS Pharmacol Transl Sci 4, 533-542.
Kline, T. B., Benington, F., Morin, R. D., Beaton, J. M., 1982. Structure-Activity Relationships in Potentially Hallucinogenic N,N-Dialkyltryptamines Substituted in the Benzene Moiety. J Med Chem 25, 908-913.
Lyon, R. A., Titeler, M., Seggel, M. R., Glennon, R. A., 1988. Indolealkylamine analogs share 5-HT2 binding characteristics with phenylalkylamine hallucinogens. Eur J Pharmacol 145, 291-297.
Macor, J. E., Fox, C. B., Johnson, C., Koe, B. K., Lebel, L. A., Zorn, S. H., 1992. 1-(2-Aminoethyl)-3-methyl-8,9-dihydropyrano[3,2-e]indole: A Rotationally Restricted Phenolic Analog of the Neurotransmitter Serotonin and Agonist Selective for Serotonin (5-HT2-Type) Receptors. J Med Chem 35, 3625-3632.
Matsushima, Y., Shirota, O., Kikura-Hanajiri, R., Goda, Y., Eguchi, F., 2009. Effects of psilocybe argentipes on marble-burying behavior in mice. Biosci Biotechnol Biochem 73, 1866-1868.
May, J. A., Chen, H. H., Rusinko, A., Lynch, V. M., Sharif, N. A., McLaughlin, M. A., 2003. A novel and selective 5-HT2 receptor agonist with ocular hypotensive activity: (S)-(+)-1-(2-aminopropyl)-8,9-dihydropyrano[3,2-e]indole. J Med Chem 46, 4188-4195.
McDonald, A. D., Perkins, L. J., Buller, A. R., 2019. Facile in Vitro Biocatalytic Production of Diverse Tryptamines. ChemBioChem 20, 1939-1944.
Miles, E. W., Phillips, R. S., 1985. Photoinactivation and Photoaffinity Labeling of Tryptophan Synthase α2β2Complex by the Product Analogue 6-Azido-L-tryptophan. Biochemistry 24, 4694-4703.
Milne, N., Thomsen, P., Molgaard Knudsen, N., Rubaszka, P., Kristensen, M., Borodina, I., 2020a. Metabolic engineering of Saccharomyces cerevisiae for the de novo production of psilocybin and related tryptamine derivatives. Metab Eng 60, 25-36.
Milne, N., Thomsen, P., Molgaard Knudsen, N., Rubaszka, P., Kristensen, M., Borodina, I., 2020b. Metabolic engineering of Saccharomyces cerevisiae for the de novo production of psilocybin and related tryptamine derivatives. Metab Eng 60, 25-36.
Murciano-Calles, J., Romney, D. K., Brinkmann-Chen, S., Buller, A. R., Arnold, F. H., 2016. A Panel of TrpB Biocatalysts Derived from Tryptophan Synthase through the Transfer of Mutations that Mimic Allosteric Activation. Angewandte Chemie International Edition 55, 11577-11581.
Phillips, R. S., 2004. Synthetic applications of tryptophan synthase. Tetrahedron Asymmetry 15, 2787-2792.
Phillips, R. S., Cohen, L. A., Annby, U., Wensbo, D., Gronowitz, S., 1995. Enzymatic synthesis of Thia-L-tryptophans. Bioorg Med Chem Lett 5, 1133-1134.
Repke, D. B., Grotjahn, D. B., Shulgin, A. T., 1985. Psychotomimetic N-Methyl-N-isopropyltryptamines. Effects of Variation of Aromatic Oxygen Substituents. J Med Chem 28, 892-896.
Rix, G., Watkins-Dulaney, E. J., Almhjell, P. J., Boville, C. E., Arnold, F. H., Liu, C. C., 2020. Scalable continuous evolution for the generation of diverse enzyme variants encompassing promiscuous activities. Nature Communications 2020 11:1 11, 1-11.
Romney, D. K., Murciano-Calles, J., Wehrmüller, J. E., Arnold, F. H., 2017. Unlocking Reactivity of TrpB: A General Biocatalytic Platform for Synthesis of Tryptophan Analogues. J Am Chem Soc 139, 10769-10776.
Runguphan, W., Qu, X., O'Connor, S E., 2010. Integrating carbon-halogen bond formation into medicinal plant metabolism. Nature 2010 468:7322 468, 461-464.
Saito, A., Rilling, H. C., 2006. Synthesis of Photoreactive Tryptophan Analogs: Enzymatic Conversion of Azidoindoles to Azidotryptophans.
Sherwood, A. M., Meisenheimer, P., Tarpley, G., Kargbo, R. B., 2020. An Improved, Practical, and Scalable Five-Step Synthesis of Psilocybin. Synthesis (Germany) 52, 688-694.
Sloan, M. J, Phillips, R. S., 1992. Enzymatic synthesis of aza-1-tryptophans: The preparation of 5- and 6-Aza-1-tryptophan. Bioorg Med Chem Lett 2, 1053-1056.
Smith, D. R. M., Willemse, T., Gkotsi, D. S., Schepens, W., Maes, B. U. W., Ballet, S., Goss, R. J. M., 2014. The first one-pot synthesis of 1-7-iodotryptophan from 7-iodoindole and serine, and an improved synthesis of other 1-7-halotryptophans. Org Lett 16, 2622-2625.
Watkins-Dulaney, E., Straathof, S., Arnold, F., 2021. Tryptophan Synthase: Biocatalyst Extraordinaire. ChemBioChem.
Welch, M., Phillips, R. S., 1999. Enzymatic syntheses of 6-(4H-Selenolo[3,2-b]pyrrolyl)-L-alanine, 4-(6H-selenolo[2,3-b]pyrrolyl)-L-alanine, and 6-(4H-furo[3,2-b]pyrrolyl)-L-alanine. Bioorg Med Chem Lett 9, 637-640.
Wilcox, M., 1974. The enzymatic synthesis of 1-tryptophan analogues. Anal Biochem 59, 436-440.
Xu, L., Han, F., Dong, Z., Wei, Z., 2020. Engineering Improves Enzymatic Synthesis of L-Tryptophan by Tryptophan Synthase from Escherichia coli. Microorganisms 2020, Vol. 8, Page 519 8, 519.
Xu, L., Li, T., Huo, Z., Chen, Q., Xia, Q., Jiang, B., 2021. Directed Evolution Improves the Enzymatic Synthesis of L-5-Hydroxytryptophan by an Engineered Tryptophan Synthase. Appl Biochem Biotechnol 193, 3407-3417.
Xu, P., Li, L., Zhang, F., Stephanopoulos, G., Koffas, M., 2014. Improving fatty acids production by engineering dynamic pathway regulation and metabolic control. Proc Natl Acad Sci USA 111, 11299-304.
Xu, P., Vansiri, A., Bhan, N., Koffas, M. A. G., 2012. ePathBrick: A Synthetic Biology Platform for Engineering Metabolic Pathways in E. coli. ACS Synth Biol 1, 256-66.

All publications and patents referred to herein are incorporated by reference. Various modifications and variations of the described subject matter will be apparent to those skilled in the art without departing from the scope and spirit of the invention. Although the invention has been described in connection with specific embodiments, it should be understood that the invention as claimed should not be unduly limited to these embodiments. Indeed, various modifications for carrying out the invention are obvious to those skilled in the art and are intended to be within the scope of the following claims.

1. A method for the production of a tryptophan, a tryptamine, or an intermediate or a side product thereof, or a derivative thereof, comprising:
contacting a first prokaryotic host cell with one or more expression vectors, wherein each expression vector comprises a gene selected from the group consisting of psiH, CPR, and combinations thereof;
contacting a second prokaryotic host cell with one or more expression vectors, wherein each expression vector comprises a gene selected from the group consisting of psiD, a psiK, psiM, and combinations thereof, and
co-culturing the first prokaryotic host cell with the second prokaryotic host cell.

2. The method of claim 1, wherein the tryptophan, tryptamine, intermediate or side product is a non-naturally occurring derivative.

3. The method of claim 1, wherein the tryptamine is a psilocybin derivative.

4. The method of claim 1, wherein at least one of the following is true:
the psiH gene encodes a polypeptide comprising the amino acid sequence of SEQ ID NO: 10 or a sequence having at least 60%, at least 70%, at least 80%, at least 90%, at least 95%, at least 96%, at least 97%, at least 98%, or at least 99% sequence identity thereto; and
the CPR gene encodes a polypeptide comprising the amino acid sequence of SEQ ID NO: 2, 4, 6, 8, or a sequence having at least 60%, at least 70%, at least 80%, at least 90%, at least 95%, at least 96%, at least 97%, at least 98%, or at least 99% sequence identity thereto.

5. (canceled)

6. The method of claim 1, wherein the first prokaryotic cell and the second prokaryotic cell are independently i-s selected from the group consisting of Escherichia coli, Corynebacterium glutamicum, Vibrio natriegens, Bacillus subtilis, Bacillus megaterium, Escherichia coli Nissle 1917, Clostridium acetobutlyicum, Streptomyces coelicolor, Lactococcus lactis, Pseudomonas putida, Streptomyces clavuligerus, and Streptomyces venezuelae.

7. (canceled)

8. The method of claim 1, wherein the first prokaryotic cell is contacted with an expression vector comprising a psiH gene, and a CPR gene, wherein each gene:
is under the control of a single promoter in operon configuration;
is under the control of a separate promoter in pseudooperon configuration, or
is under the control of a separate promoter in monocistronic configuration.

9. The method of claim 8, wherein the promoter is selected from the group consisting of G6 mutant T7, H9 mutant T7, H10 mutant T7, C4 mutant T7, consensus T7, Lac, Lac UV5, tac, trc, GAP, and xylA promoter.

10-11. (canceled)

12. The method of claim 1, wherein the second prokaryotic cell is contacted with an expression vector comprising a psiD gene, a psiK gene, and a psiM gene, wherein each gene:
is under the control of a single promoter in operon configuration;
is under the control of a separate promoter in pseudooperon configuration, or
is under the control of a separate promoter in monocistronic configuration.

13. The method of claim 12, wherein the promoter is selected from the group consisting of G6 mutant T7, H9 mutant T7, H10 mutant T7, C4 mutant T7, consensus T7, Lac, Lac UV5, tac, trc, GAP, and xylA promoter.

14-15. (canceled)

16. The method of claim 1, wherein the intermediate or side product is norbaeocystin, baeocystin, 4-hydroxytryptophan, 4-hydroxytryptamine, aeruginascin, psilocin, norpsilocin, or 4-hydroxy-N,N,N-trimethyltryptamine (4-OH-TMT).

17. The method of claim 1, wherein the co-culture is supplemented with a supplement independently selected from the group consisting of serine, tryptamine, tryptamine derivatives, tryptophan, tryptophan derivatives, indole, indole derivatives, and combinations thereof, and derivatives thereof.

18. The method of claim 17, wherein the supplement is fed continuously to the host cell co-culture.

19. The method of claim 1, wherein the co-culture is actively growing.

20-25. (canceled)

26. A recombinant prokaryotic cell comprising one or more expression vectors, wherein each expression vector comprises a gene selected from the group consisting of psiH, CPR, and combinations thereof.

27. The recombinant prokaryotic cell of claim 26, wherein at least one of the following is true:
the psiH gene encodes a polypeptide comprising the amino acid sequence of SEQ ID NO: 10 or a sequence having at least 60%, at least 70%, at least 80%, at least 90%, at least 95%, at least 96%, at least 97%, at least 98%, or at least 99% sequence identity thereto; and
the CPR gene encodes a polypeptide comprising the amino acid sequence of SEQ ID NO: 2, 4, 6, 8, or a sequence having at least 60%, at least 70%, at least 80%, at least 90%, at least 95%, at least 96%, at least 97%, at least 98%, or at least 99% sequence identity thereto.

28. (canceled)

29. The recombinant prokaryotic cell of claim 26, wherein the prokaryotic cell is selected from the group consisting of Escherichia coli, Corynebacterium glutamicum, Vibrio natriegens, Bacillus subtilis, Bacillus megaterium, Escherichia coli Nissle 1917, Clostridium acetobutlyicum, Streptomyces coelicolor, Lactococcus lactis, Pseudomonas putida, Streptomyces clavuligerus, and Streptomyces venezuelae.

30. The recombinant prokaryotic cell of claim 26, wherein the expression vector comprises a psiH gene, and a CPR gene, wherein each gene:
is under the control of a single promoter in operon configuration;
is under control of a separate promoter in pseudooperon configuration; or
is under control of a separate promoter in monocistronic configuration.

31. The recombinant prokaryotic cell of claim 30, wherein the promoter is selected from the group consisting of G6 mutant T7, H9 mutant T7, H10 mutant T7, C4 mutant T7, consensus T7, Lac, Lac UV5, tac, trc, GAP, and xylA promoter.

32-41. (canceled)

42. An expression vector comprising a psiH gene, and a CPR gene, wherein each gene:
is under the control of a single promoter in operon configuration,
is under the control of a separate promoter in pseudooperon configuration: or
is under the control of a separate promoter in monocistronic configuration.

43. The expression vector of claim 42, wherein the promoter is selected from the group consisting of G6 mutant T7, H9 mutant T7, H10 mutant T7, C4 mutant T7, consensus T7, Lac, Lac UV5, tac, trc, GAP, and xylA promoter.

44-61. (canceled)


SEQUENCE LISTING (73 sequences)

SEQ ID NO: 1 - DNA, 2223 bp, synthetic construct
  source 1..2223: mol_type = other DNA
atggcactattgttagctgtatttaggagaggaggttctgcgagctctagcagcgatgtttttgtactaggtttgggtgtcgtcctggcggcgctctacatctttcgtgatcaactgttcgccgcaagcaagccgaaagtggcaccggtttcgaccacgaaaccggcgaatggtagcgcgaacccgcgcgactttatcgctaagatgaaacaaggtaagaaacgcatcgtgattttctatggctcgcaaaccggtacggctgaagaatacgcaattcgtctagcgaaagaggcaaagcagaaattcggcttggcgagcttggtttgtgatccggaagaatacgatttcgaaaaactggaccagctgccggaggacagcattgcgttctttgtcgttgcgacctacggcgagggtgagccgaccgataatgcagtccaactgctgcaaaacttgcaagatgaaagctttgaattttcctctggtgagcgcaagttgtctggtctgaagtacgtggtctttggtctgggaaacaaaacgtacgaacattacaatctgatcggtcgcaccgtggacgctcagttggcgaagatgggtgcaatccgcatcggcgagcgtggcgagggtgacgacgacaaatccatggaagaggattatctcgaatggaaagatggcatgtgggaagcgttcgctaccgcaatgggcgtggaggagggccagggtggcgacagcgctgatttcgtcgtttccgaactggaatcacatccaccggaaaaagtttatcagggtgagttctccgctcgggcattgaccaaaaccaagggcattcacgatgcgaaaaacccgttcgcggccccgatcgccgtggcgcgtgaacttttccagtccgtcgttgaccgtaattgtgtgcatgtggagttcaacatcgaaggctctggtatcacctaccagcatggtgaccacgtgggtctgtggccgctgaacccggacgtcgaagtggagcgcttgctttgcgttttgggcctggcggaaaaacgtgacgcagttattagcatcgagtcgctggacccggcgcttgccaaggtgccatttccggtgccgactacgtacggcgcggtgctgcgccactacattgatatcagcgcggtggctggtcgtcaaattctgggcaccctgagcaagttcgcaccgactccggaagctgaggccttcctgcgaaatttaaataccaataaagaagagtaccataacgttgtggcgaacggttgcctgaagctcggcgagatcctgcagattgcgaccggcaacgatattaccgttccgcctacgaccgcgaacactaccaaatggcccatcccgtttgatattattgttagcgcgatcccgagattgcaaccgcgttattattccatctccagcagcccgaaaatccacccgaataccattcacgcgacggttgtcgtgctgaagtacgagaacgtgccaactgagcctatcccgcgtaaatgggtttacggcgttggcagcaattttttgctgaacctgaagtatgccgtgaacaaagagccggttccgtacatcacccagaacggcgagcagcgtgtgggtgtaccggaatacctgatagcgggtccgcgtggtagctataagaccgaatcgttctacaaggccccaatccacgttcgtcgttctacctttcgtttaccgaccaacccgaagtccccggtgatcatgattggaccgggtacaggtgtggctccgttccgcggttttgttcaagaacgcgtggcgctggcccgtcgtagcatcgagaagaacggcccagattccctcgcagattggggtcgcatcagcctgttttacggctgccgtcgtagtgatgaagacttcctgtacaaggacgaatggccgcagtatgaggcggagctgaaaggcaagtttaaactgcactgcgcattcagccgtcaaaactataagccggacggcagtaaaatttatgttcaggacctgatttgggaagatcgcgagcatattgcggacgccatcctgaatggtaagggttatgtttatatctgcggtgaagccaagagcatgagcaagcaggttgaggaggttttggctaagattctgggggaggcgaagggtggctctggtccggtagagggcgttgcggaggtgaaattgctgaaagaaagatctcgtctgatgctggacgtgtggagctaa

SEQ ID NO: 2 - protein, 740 aa, synthetic construct
MALLLAVFRRGGSASSSSDVFVLGLGVVLAALYIFRDQLFAASKPKVAPVSTTKPANGSANPRDFIAKMKQGKKRIVIFYGSQTGTAEEYAIRLAKEAKQKFGLASLVCDPEEYDFEKLDQLPEDSIAFFVVATYGEGEPTDNAVQLLQNLQDESFEFSSGERKLSGLKYVVFGLGNKTYEHYNLIGRTVDAQLAKMGAIRIGERGEGDDDKSMEEDYLEWKDGMWEAFATAMGVEEGQGGDSADFVVSELESHPPEKVYQGEFSARALTKTKGIHDAKNPFAAPIAVARELFQSVVDRNCVHVEFNIEGSGITYQHGDHVGLWPLNPDVEVERLLCVLGLAEKRDAVISIESLDPALAKVPFPVPTTYGAVLRHYIDISAVAGRQILGTLSKFAPTPEAEAFLRNLNTNKEEYHNVVANGCLKLGEILQIATGNDITVPPTTANTTKWPIPFDIIVSAIPRLQPRYYSISSSPKIHPNTIHATVVVLKYENVPTEPIPRKWVYGVGSNFLLNLKYAVNKEPVPYITQNGEQRVGVPEYLIAGPRGSYKTESFYKAPIHVRRSTFRLPTNPKSPVIMIGPGTGVAPFRGFVQERVALARRSIEKNGPDSLADWGRISLFYGCRRSDEDFLYKDEWPQYEAELKGKFKLHCAFSRQNYKPDGSKIYVQDLIWEDREHIADAILNGKGYVYICGEAKSMSKQVEEVLAKILGEAKGGSGPVEGVAEVKLLKERSRLMLDVWS

SEQ ID NO: 3 - DNA, 2187 bp, synthetic construct
  source 1..2187: mol_type = other DNA
atggcgagctctagcagcgatgtttttgtactaggtttgggtgtcgtcctggcggcgctctacatctttcgtgatcaactgttcgccgcaagcaagccgaaagtggcaccggtttcgaccacgaaaccggcgaatggtagcgcgaacccgcgcgactttatcgctaagatgaaacaaggtaagaaacgcatcgtgattttctatggctcgcaaaccggtacggctgaagaatacgcaattcgtctagcgaaagaggcaaagcagaaattcggcttggcgagcttggtttgtgatccggaagaatacgatttcgaaaaactggaccagctgccggaggacagcattgcgttctttgtcgttgcgacctacggcgagggtgagccgaccgataatgcagtccaactgctgcaaaacttgcaagatgaaagctttgaattttcctctggtgagcgcaagttgtctggtctgaagtacgtggtctttggtctgggaaacaaaacgtacgaacattacaatctgatcggtcgcaccgtggacgctcagttggcgaagatgggtgcaatccgcatcggcgagcgtggcgagggtgacgacgacaaatccatggaagaggattatctcgaatggaaagatggcatgtgggaagcgttcgctaccgcaatgggcgtggaggagggccagggtggcgacagcgctgatttcgtcgtttccgaactggaatcacatccaccggaaaaagtttatcagggtgagttctccgctcgggcattgaccaaaaccaagggcattcacgatgcgaaaaacccgttcgcggccccgatcgccgtggcgcgtgaacttttccagtccgtcgttgaccgtaattgtgtgcatgtggagttcaacatcgaaggctctggtatcacctaccagcatggtgaccacgtgggtctgtggccgctgaacccggacgtcgaagtggagcgcttgctttgcgttttgggcctggcggaaaaacgtgacgcagttattagcatcgagtcgctggacccggcgcttgccaaggtgccatttccggtgccgactacgtacggcgcggtgctgcgccactacattgatatcagcgcggtggctggtcgtcaaattctgggcaccctgagcaagttcgcaccgactccggaagctgaggccttcctgcgaaatttaaataccaataaagaagagtaccataacgttgtggcgaacggttgcctgaagctcggcgagatcctgcagattgcgaccggcaacgatattaccgttccgcctacgaccgcgaacactaccaaatggcccatcccgtttgatattattgttagcgcgatcccgagattgcaaccgcgttattattccatctccagcagcccgaaaatccacccgaataccattcacgcgacggttgtcgtgctgaagtacgagaacgtgccaactgagcctatcccgcgtaaatgggtttacggcgttggcagcaattttttgctgaacctgaagtatgccgtgaacaaagagccggttccgtacatcacccagaacggcgagcagcgtgtgggtgtaccggaatacctgatagcgggtccgcgtggtagctataagaccgaatcgttctacaaggccccaatccacgttcgtcgttctacctttcgtttaccgaccaacccgaagtccccggtgatcatgattggaccgggtacaggtgtggctccgttccgcggttttgttcaagaacgcgtggcgctggcccgtcgtagcatcgagaagaacggcccagattccctcgcagattggggtcgcatcagcctgttttacggctgccgtcgtagtgatgaagacttcctgtacaaggacgaatggccgcagtatgaggcggagctgaaaggcaagtttaaactgcactgcgcattcagccgtcaaaactataagccggacggcagtaaaatttatgttcaggacctgatttgggaagatcgcgagcatattgcggacgccatcctgaatggtaagggttatgtttatatctgcggtgaagccaagagcatgagcaagcaggttgaggaggttttggctaagattctgggggaggcgaagggtggctctggtccggtagagggcgttgcggaggtgaaattgctgaaagaaagatctcgtctgatgctggacgtgtggagctaa

SEQ ID NO: 4 - protein, 728 aa, synthetic construct
MASSSSDVFVLGLGVVLAALYIFRDQLFAASKPKVAPVSTTKPANGSANPRDFIAKMKQGKKRIVIFYGSQTGTAEEYAIRLAKEAKQKFGLASLVCDPEEYDFEKLDQLPEDSIAFFVVATYGEGEPTDNAVQLLQNLQDESFEFSSGERKLSGLKYVVFGLGNKTYEHYNLIGRTVDAQLAKMGAIRIGERGEGDDDKSMEEDYLEWKDGMWEAFATAMGVEEGQGGDSADFVVSELESHPPEKVYQGEFSARALTKTKGIHDAKNPFAAPIAVARELFQSVVDRNCVHVEFNIEGSGITYQHGDHVGLWPLNPDVEVERLLCVLGLAEKRDAVISIESLDPALAKVPFPVPTTYGAVLRHYIDISAVAGRQILGTLSKFAPTPEAEAFLRNLNTNKEEYHNVVANGCLKLGEILQIATGNDITVPPTTANTTKWPIPFDIIVSAIPRLQPRYYSISSSPKIHPNTIHATVVVLKYENVPTEPIPRKWVYGVGSNFLLNLKYAVNKEPVPYITQNGEQRVGVPEYLIAGPRGSYKTESFYKAPIHVRRSTFRLPTNPKSPVIMIGPGTGVAPFRGFVQERVALARRSIEKNGPDSLADWGRISLFYGCRRSDEDFLYKDEWPQYEAELKGKFKLHCAFSRQNYKPDGSKIYVQDLIWEDREHIADAILNGKGYVYICGEAKSMSKQVEEVLAKILGEAKGGSGPVEGVAEVKLLKERSRLMLDVWS

SEQ ID NO: 5 - DNA, 1956 bp, synthetic construct
  source 1..1956: mol_type = other DNA
atggcactattgttagctgtatttaggagaggaggttctgacacgcagaaaccggcggtgcgtccgaccccgctggtgaaggaagaggacgaagaggaggaagacgatagcgcaaagaagaaggtgactatctttttcggcacccaaaccggcactgccgaaggttttgccaaggctctcgccgaggaagcgaaagcgcgttatgaaaaggcagtctttaaagttgttgacctggacaactatgcggctgacgatgagcagtatgaggagaaattgaagaaggaaaagttagcattctttatgctggctacctacggcgacggcgaaccgacggataatgcggcgcgtttctataaatggttcctggaaggtaaggagcgcgagccgtggctgagcgacctgacgtatggtgtgttcggtttggggaaccgtcagtatgagcactttaataaagttgcgaaggcggttgatgaggtgttgattgaacaaggtgcgaagagattggttccggtcggtctgggcgatgacgaccagtgtattgaggacgattttaccgcgtggcgtgagcaggtttggccggagttggaccagctgctgcgtgacgaggatgatgaaccgacttctgcgaccccttataccgcagctatcccggaataccgtgtcgaaatttacgactccgtggttagcgtgtacgaggagacccatgcgcttaagcagaacggtcaggcagtttatgatattcaccatccgtgtcgttctaacgttgcggtgcgtcgtgagctgcataccccgctgtccgatcgttcttgtattcacctggaattcgatatcagcgacaccggtctgatctacgagaccggcgaccacgtgggtgtccacacggaaaacagcattgaaacagtagaggaggcagcgaaattgttaggctaccagctggatacgattttcagcgttcatggtgataaagaagacggcaccccgctgggtggctctagcctgccgccgccatttccgggtccgtgcaccctgcgtaccgccttggcgcgttacgcagacttgctgaatccgcctcgtaaagcggcgtttctggcgctggcggcgcatgcgtcagacccggcagaggccgaacgtctgaaatttctgagttccccggcgggtaaagatgaatattctcaatgggttaccgcgtcccaacgtagcctgcttgagattatggccgagtttccgagcgcgaagccgccgttaggtgtgttctttgcagcgatcgcaccgcgcctgcaaccgcgctactatagcatctccagctctccgcgtttcgctccgtcccgcatccacgttacctgcgccttagtctacggtccaagtccgaccggtcgcattcacaagggtgtgtgcagcaactggatgaaaaacagcctgccgagcgaggaaacgcatgattgcagctgggcaccagttttcgtgcgccaaagcaactttaagcttccggcggattcgaccacccccatcgtaatggtcggtccaggtacgggcttcgctccttttagaggttttctgcaagaacgcgcaaagctgcaagaagctggtgaaaaattgggtccggcggttctgttcttcgggtgccgtaatcgccagatggattacatttacgaggacgaactgaagggctacgtcgagaaaggtattctgaccaatctgatcgtggccttcagccgtgaaggcgcgaccaaagagtatgtgcagcataaaatgttggagaaggcttcggacacgtggagcttaatcgcccagggcggctacctgtatgtttgcggtgatgcaaaaggcatggcgcgcgatgttcaccgtaccctgcacaccatcgttcaggagcaagaaagcgtggattcttcgaaagctgagttcttggtgaaaaagttgcaaatggatggccgctacctgcgtgacatctggtaa

SEQ ID NO: 6 - protein, 651 aa, synthetic construct
MALLLAVFRRGGSDTQKPAVRPTPLVKEEDEEEEDDSAKKKVTIFFGTQTGTAEGFAKALAEEAKARYEKAVFKVVDLDNYAADDEQYEEKLKKEKLAFFMLATYGDGEPTDNAARFYKWFLEGKEREPWLSDLTYGVFGLGNRQYEHFNKVAKAVDEVLIEQGAKRLVPVGLGDDDQCIEDDFTAWREQVWPELDQLLRDEDDEPTSATPYTAAIPEYRVEIYDSVVSVYEETHALKQNGQAVYDIHHPCRSNVAVRRELHTPLSDRSCIHLEFDISDTGLIYETGDHVGVHTENSIETVEEAAKLLGYQLDTIFSVHGDKEDGTPLGGSSLPPPFPGPCTLRTALARYADLLNPPRKAAFLALAAHASDPAEAERLKFLSSPAGKDEYSQWVTASQRSLLEIMAEFPSAKPPLGVFFAAIAPRLQPRYYSISSSPRFAPSRIHVTCALVYGPSPTGRIHKGVCSNWMKNSLPSEETHDCSWAPVFVRQSNFKLPADSTTPIVMVGPGTGFAPFRGFLQERAKLQEAGEKLGPAVLFFGCRNRQMDYIYEDELKGYVEKGILTNLIVAFSREGATKEYVQHKMLEKASDTWSLIAQGGYLYVCGDAKGMARDVHRTLHTIVQEQESVDSSKAEFLVKKLQMDGRYLRDIW

SEQ ID NO: 7 - DNA, 1920 bp, synthetic construct
  source 1..1920: mol_type = other DNA
atggacacgcagaaaccggcggtgcgtccgaccccgctggtgaaggaagaggacgaagaggaggaagacgatagcgcaaagaagaaggtgactatctttttcggcacccaaaccggcactgccgaaggttttgccaaggctctcgccgaggaagcgaaagcgcgttatgaaaaggcagtctttaaagttgttgacctggacaactatgcggctgacgatgagcagtatgaggagaaattgaagaaggaaaagttagcattctttatgctggctacctacggcgacggcgaaccgacggataatgcggcgcgtttctataaatggttcctggaaggtaaggagcgcgagccgtggctgagcgacctgacgtatggtgtgttcggtttggggaaccgtcagtatgagcactttaataaagttgcgaaggcggttgatgaggtgttgattgaacaaggtgcgaagagattggttccggtcggtctgggcgatgacgaccagtgtattgaggacgattttaccgcgtggcgtgagcaggtttggccggagttggaccagctgctgcgtgacgaggatgatgaaccgacttctgcgaccccttataccgcagctatcccggaataccgtgtcgaaatttacgactccgtggttagcgtgtacgaggagacccatgcgcttaagcagaacggtcaggcagtttatgatattcaccatccgtgtcgttctaacgttgcggtgcgtcgtgagctgcataccccgctgtccgatcgttcttgtattcacctggaattcgatatcagcgacaccggtctgatctacgagaccggcgaccacgtgggtgtccacacggaaaacagcattgaaacagtagaggaggcagcgaaattgttaggctaccagctggatacgattttcagcgttcatggtgataaagaagacggcaccccgctgggtggctctagcctgccgccgccatttccgggtccgtgcaccctgcgtaccgccttggcgcgttacgcagacttgctgaatccgcctcgtaaagcggcgtttctggcgctggcggcgcatgcgtcagacccggcagaggccgaacgtctgaaatttctgagttccccggcgggtaaagatgaatattctcaatgggttaccgcgtcccaacgtagcctgcttgagattatggccgagtttccgagcgcgaagccgccgttaggtgtgttctttgcagcgatcgcaccgcgcctgcaaccgcgctactatagcatctccagctctccgcgtttcgctccgtcccgcatccacgttacctgcgccttagtctacggtccaagtccgaccggtcgcattcacaagggtgtgtgcagcaactggatgaaaaacagcctgccgagcgaggaaacgcatgattgcagctgggcaccagttttcgtgcgccaaagcaactttaagcttccggcggattcgaccacccccatcgtaatggtcggtccaggtacgggcttcgctccttttagaggttttctgcaagaacgcgcaaagctgcaagaagctggtgaaaaattgggtccggcggttctgttcttcgggtgccgtaatcgccagatggattacatttacgaggacgaactgaagggctacgtcgagaaaggtattctgaccaatctgatcgtggccttcagccgtgaaggcgcgaccaaagagtatgtgcagcataaaatgttggagaaggcttcggacacgtggagcttaatcgcccagggcggctacctgtatgtttgcggtgatgcaaaaggcatggcgcgcgatgttcaccgtaccctgcacaccatcgttcaggagcaagaaagcgtggattcttcgaaagctgagttcttggtgaaaaagttgcaaatggatggccgctacctgcgtgacatctggtaa

SEQ ID NO: 8 - protein, 639 aa, synthetic construct
MDTQKPAVRPTPLVKEEDEEEEDDSAKKKVTIFFGTQTGTAEGFAKALAEEAKARYEKAVFKVVDLDNYAADDEQYEEKLKKEKLAFFMLATYGDGEPTDNAARFYKWFLEGKEREPWLSDLTYGVFGLGNRQYEHFNKVAKAVDEVLIEQGAKRLVPVGLGDDDQCIEDDFTAWREQVWPELDQLLRDEDDEPTSATPYTAAIPEYRVEIYDSVVSVYEETHALKQNGQAVYDIHHPCRSNVAVRRELHTPLSDRSCIHLEFDISDTGLIYETGDHVGVHTENSIETVEEAAKLLGYQLDTIFSVHGDKEDGTPLGGSSLPPPFPGPCTLRTALARYADLLNPPRKAAFLALAAHASDPAEAERLKFLSSPAGKDEYSQWVTASQRSLLEIMAEFPSAKPPLGVFFAAIAPRLQPRYYSISSSPRFAPSRIHVTCALVYGPSPTGRIHKGVCSNWMKNSLPSEETHDCSWAPVFVRQSNFKLPADSTTPIVMVGPGTGFAPFRGFLQERAKLQEAGEKLGPAVLFFGCRNRQMDYIYEDELKGYVEKGILTNLIVAFSREGATKEYVQHKMLEKASDTWSLIAQGGYLYVCGDAKGMARDVHRTLHTIVQEQESVDSSKAEFLVKKLQMDGRYLRDIW

SEQ ID NO: 9 - DNA, 1527 bp, synthetic construct
  source 1..1527: mol_type = other DNA
atgatcgctgtactattctccttcgtcattgcaggatgcatatactacatcgtttctcgtagagtgaggcggtcgcgcttgccaccagggccgcctggcattcctattcccttcattgggaacatgtttgatatgcctgaagaatctccatggttaacatttctacaatggggacgggattacaacaccgatattctctacgtggatgctggagggacagaaatggttattcttaacacgttggagaccattaccgatctattagaaaagcgagggtccatttattctggccgacttgagagtacaatggtcaacgaacttatggggtgggagtttgacttagggttcatcacatacggcgacaggtggcgcgaagaaaggcgcatgttcgccaaggagttcagtgagaagggcatcaagcaatttcgccatgctcaagtgaaagctgcccatcagcttgtccaacagcttaccaaaacgccagaccgctgggcacaacatattcgccatcagatagcggcaatgtcactggatattggttatggaattgatcttgcagaagacgacccttggctggaagcgacccatttggctaatgaaggcctcgccatagcatcagtgccgggcaaattttgggtcgattcgttcccttctctaaaataccttcctgcttggttcccaggtgctgtcttcaagcgcaaagcgaaggtctggcgagaagccgccgaccacatggttgacatgccttatgaaactatgaggaaattagcacctcaaggattgactcgtccgtcgtatgcttcagctcgtctgcaagccatggatctcaacggtgaccttgagcatcaagaacacgtaatcaagaacacagccgcagaggttaatgtcggtggaggcgatactactgtctctgctatgtctgcgttcatcttggccatggtgaagtaccctgaggtccagcgaaaggttcaagcggagcttgatgctctgaccaataacggccaaattcctgactatgacgaagaagatgactccttgccatacctcaccgcatgtatcaaggagcttttccggtggaatcaaatcgcacccctcgctataccgcacaaattaatgaaggacgacgtgtaccgcgggtatctgattcccaagaacactctagtcttcgcaaacacctgggcagtattaaacgatccagaagtctatccagatccctctgtgttccgcccagaaagatatcttggtcctgacgggaagcctgataacactgtacgcgacccacgtaaagcggcatttggctatggacgacgaaattgtcccggaattcatctagcgcagtcgacggtttggattgcaggggcaaccctcttatcagcgttcaatatcgagcgacctgtcgatcagaatgggaagcccattgacataccggctgattttactacaggattcttcagacacccagtgcctttccagtgcaggtttgttcctcgaacagagcaagtctcacagtcggtatccggaccctga

SEQ ID NO: 10 - protein, 508 aa, synthetic construct
MIAVLFSFVIAGCIYYIVSRRVRRSRLPPGPPGIPIPFIGNMFDMPEESPWLTFLQWGRDYNTDILYVDAGGTEMVILNTLETITDLLEKRGSIYSGRLESTMVNELMGWEFDLGFITYGDRWREERRMFAKEFSEKGIKQFRHAQVKAAHQLVQQLTKTPDRWAQHIRHQIAAMSLDIGYGIDLAEDDPWLEATHLANEGLAIASVPGKFWVDSFPSLKYLPAWFPGAVFKRKAKVWREAADHMVDMPYETMRKLAPQGLTRPSYASARLQAMDLNGDLEHQEHVIKNTAAEVNVGGGDTTVSAMSAFILAMVKYPEVQRKVQAELDALTNNGQIPDYDEEDDSLPYLTACIKELFRWNQIAPLAIPHKLMKDDVYRGYLIPKNTLVFANTWAVLNDPEVYPDPSVFRPERYLGPDGKPDNTVRDPRKAAFGYGRRNCPGIHLAQSTVWIAGATLLSAFNIERPVDQNGKPIDIPADFTTGFFRHPVPFQCRFVPRTEQVSQSVSGP

SEQ ID NO: 11 - DNA, 1320 bp, synthetic construct
  source 1..1320: mol_type = other DNA
atgcaggtgatacccgcgtgcaactcggcagcaataagatcactatgtcctactcccgagtcttttagaaacatgggatggctctctgtcagcgatgcggtctacagcgagttcataggagagttggctacccgcgcttccaatcgaaattactccaacgagttcggcctcatgcaacctatccaggaattcaaggctttcattgaaagcgacccggtggtgcaccaagaatttattgacatgttcgagggcattcaggactctccaaggaattatcaggaactatgtaatatgttcaacgatatctttcgcaaagctcccgtctacggagaccttggccctcccgtttatatgattatggccaaattaatgaacacccgagcgggcttctctgcattcacgagacaaaggttgaaccttcacttcaaaaaacttttcgatacctggggattgttcctgtcttcgaaagattctcgaaatgttcttgtggccgaccagttcgacgacagacattgcggctggttgaacgagcgggccttgtctgctatggttaaacattacaatggacgcgcatttgatgaagtcttcctctgcgataaaaatgccccatactacggcttcaactcttacgacgacttctttaatcgcagatttcgaaaccgagatatcgaccgacctgtagtcggtggagttaacaacaccaccctcatttctgctgcttgcgaatcactttcctacaacgtctcttatgacgtccagtctctcgacactttagttttcaaaggagagacttattcgcttaagcatttgctgaataatgaccctttcaccccacaattcgagcatgggagtattctacaaggattcttgaacgtcaccgcttaccaccgatggcacgcacccgtcaatgggacaatcgtcaaaatcatcaacgttccaggtacctactttgcgcaagccccgagcacgattggcgaccctatcccggataacgattacgacccacctccttaccttaagtctcttgtctacttctctaatattgccgcaaggcaaattatgtttattgaagccgacaacaaggaaattggcctcattttccttgtgttcatcggcatgaccgaaatctcgacatgtgaagccacggtgtccgaaggtcaacacgtcaatcgtggcgatgacttgggaatgttccatttcggtggttcttcgttcgcgcttggtctgaggaaggattgcagggcagagatcgttgaaaagttcaccgaacccggaacagtgatcagaatcaacgaagtcgtcgctgctctaaaggcttag

SEQ ID NO: 12 - DNA, 1089 bp, synthetic construct
  source 1..1089: mol_type = other DNA
atggcgttcgatctcaagactgaagacggcctcatcacatatctcactaaacatctttctttggacgtcgacacgagcggagtgaagcgccttagcggaggctttgtcaatgtaacctggcgcattaagctcaatgctccttatcaaggtcatacgagcatcatcctgaagcatgctcagccgcacatgtctacggatgaggattttaagataggtgtagaacgttcggtttacgaataccaggctatcaagctcatgatggccaatcgggaggttctgggaggcgtggatggcatagtttctgtgccagaaggcctgaactacgacttagagaataatgcattgatcatgcaagatgtcgggaagatgaagacccttttagattatgtcaccgccaaaccgccacttgcgacggatatagcccgccttgttgggacagaaattggggggttcgttgccagactccataacataggccgcgagaggcgagacgatcctgagttcaaattcttctctggaaatattgtcggaaggacgacttcagaccagctgtatcaaaccatcatacccaacgcagcgaaatatggcgtcgatgaccccttgctgcctactgtggttaaggaccttgtggacgatgtcatgcacagcgaagagacccttgtcatggcggacctgtggagtggaaatattcttctccagttggaggagggaaacccatcgaagctgcagaagatatatatcctggattgggaactttgcaagtacggcccagcgtcgttggacctgggctatttcttgggtgactgctatttgatatcccgctttcaagacgagcaggtcggtacgacgatgcggcaagcctacttgcaaagctatgcgcgtacgagcaagcattcgatcaactacgccaaagtcactgcaggtattgctgctcatattgtgatgtggaccgactttatgcagtgggggagcgaggaagaaaggataaattttgtgaaaaagggggtagctgcctttcacgacgccaggggcaacaacgacaatggggaaattacgtctaccttactgaaggaatcatccactgcgtaa

SEQ ID NO: 13 - DNA, 930 bp, synthetic construct
  source 1..930: mol_type = other DNA
atgcatatcagaaatccttaccgtacaccaattgactatcaagcactttcagaggccttccctcccctcaagccatttgtgtctgtcaatgcagatggtaccagttctgttgacctcactatcccagaagcccagagggcgttcacggccgctcttcttcatcgtgacttcgggctcaccatgaccataccagaagaccgtctgtgcccaacagtccccaataggttgaactacgttctgtggattgaagatattttcaactacacgaacaaaaccctcggcctgtcggatgaccgtcctattaaaggcgttgatattggtacaggagcctccgcaatttatcctatgcttgcctgtgctcggttcaaggcatggtctatggttggaacagaggtcgagaggaagtgcattgacacggcccgcctcaatgtcgtcgcgaacaatctccaagaccgtctctcgatattagagacatccattgatggtcctattctcgtccccattttcgaggcgactgaagaatacgaatacgagtttactatgtgtaaccctccattctacgacggtgctgccgatatgcagacttcggatgctgccaaaggatttggatttggcgtgggcgctccccattctggaacagtcatcgaaatgtcgactgagggaggtgaatcggctttcgtcgctcagatggtccgtgagagcttgaagcttcgaacacgatgcagatggtacacgagtaacttgggaaagctgaaatccttgaaagaaatagtggggctgctgaaagaacttgagataagcaactatgccattaacgaatacgttcaggggtccacacgtcgttatgccgttgcgtggtctttcactgatattcaactgcctgaggagctttctcgtccctctaaccccgagctcagctctcttttctag

SEQ ID NO: 14 - protein, 439 aa, synthetic construct
MQVIPACNSAAIRSLCPTPESFRNMGWLSVSDAVYSEFIGELATRASNRNYSNEFGLMQPIQEFKAFIESDPVVHQEFIDMFEGIQDSPRNYQELCNMFNDIFRKAPVYGDLGPPVYMIMAKLMNTRAGFSAFTRQRLNLHFKKLFDTWGLFLSSKDSRNVLVADQFDDRHCGWLNERALSAMVKHYNGRAFDEVFLCDKNAPYYGFNSYDDFFNRRFRNRDIDRPVVGGVNNTTLISAACESLSYNVSYDVQSLDTLVFKGETYSLKHLLNNDPFTPQFEHGSILQGFLNVTAYHRWHAPVNGTIVKIINVPGTYFAQAPSTIGDPIPDNDYDPPPYLKSLVYFSNIAARQIMFIEADNKEIGLIFLVFIGMTEISTCEATVSEGQHVNRGDDLGMFHFGGSSFALGLRKDCRAEIVEKFTEPGTVIRINEVVAALKA

SEQ ID NO: 15 - protein, 362 aa, synthetic construct
MAFDLKTEDGLITYLTKHLSLDVDTSGVKRLSGGFVNVTWRIKLNAPYQGHTSIILKHAQPHMSTDEDFKIGVERSVYEYQAIKLMMANREVLGGVDGIVSVPEGLNYDLENNALIMQDVGKMKTLLDYVTAKPPLATDIARLVGTEIGGFVARLHNIGRERRDDPEFKFFSGNIVGRTTSDQLYQTIIPNAAKYGVDDPLLPTVVKDLVDDVMHSEETLVMADLWSGNILLQLEEGNPSKLQKIYILDWELCKYGPASLDLGYFLGDCYLISRFQDEQVGTTMRQAYLQSYARTSKHSINYAKVTAGIAAHIVMWTDFMQWGSEEERINFVKKGVAAFHDARGNNDNGEITSTLLKESSTA

SEQ ID NO: 16 - protein, 309 aa, synthetic construct
MHIRNPYRTPIDYQALSEAFPPLKPFVSVNADGTSSVDLTIPEAQRAFTAALLHRDFGLTMTIPEDRLCPTVPNRLNYVLWIEDIFNYTNKTLGLSDDRPIKGVDIGTGASAIYPMLACARFKAWSMVGTEVERKCIDTARLNVVANNLQDRLSILETSIDGPILVPIFEATEEYEYEFTMCNPPFYDGAADMQTSDAAKGFGFGVGAPHSGTVIEMSTEGGESAFVAQMVRESLKLRTRCRWYTSNLGKLKSLKEIVGLLKELEISNYAINEYVQGSTRRYAVAWSFTDIQLPEELSRPSNPELSSLF

SEQ ID NO: 17 - DNA, 23 bp, synthetic construct
  source 1..23: mol_type = other DNA
taatacgactcactacggaagaa

SEQ ID NO: 18 - DNA, 23 bp, synthetic construct
  source 1..23: mol_type = other DNA
taatacgactcactatttcggaa

SEQ ID NO: 19 - DNA, 23 bp, synthetic construct
  source 1..23: mol_type = other DNA
taatacgactcactaatactgaa

SEQ ID NO: 20 - DNA, 23 bp, synthetic construct
  source 1..23: mol_type = other DNA
taatacgactcactatcaaggaa

SEQ ID NO: 21 - DNA, 23 bp, synthetic construct
  source 1..23: mol_type = other DNA
taatacgactcactataggggaa

SEQ ID NO: 22 - DNA, 31 bp, synthetic construct
  source 1..31: mol_type = other DNA
tttacactttatgcttccggctcgtatgttg

SEQ ID NO: 23 - DNA, 31 bp, synthetic construct
  source 1..31: mol_type = other DNA
tttacactttatgcttccggctcgtataatg

SEQ ID NO: 24 - DNA, 29 bp, synthetic construct
  source 1..29: mol_type = other DNA
ttgacaattaatcatcggctcgtataatg

SEQ ID NO: 25 - DNA, 30 bp, synthetic construct
  source 1..30: mol_type = other DNA
ttgacaattaatcatccggctcgtataatg

SEQ ID NO: 26 - DNA, 101 bp, synthetic construct
  source 1..101: mol_type = other DNA
gcgtaatgcttaggcacaggattgatttgtcgcaatgattgacacgattccgcttgacgctgcgtaaggtttttgtaattttacaggcaaccttttattca

SEQ ID NO: 27 - DNA, 92 bp, synthetic construct
  source 1..92: mol_type = other DNA
ttgaaataaacatttattttgtatatgatgagataaagttagtttattggataaacaaactaactcaattaagatagttgatggataaactt

SEQ ID NO: 28 - DNA, 8848 bp, synthetic construct
  source 1..8848: mol_type = other DNA
taatacgactcactacggaagaattgtgagcggataacaattcccctctagaaataattttgtttaactttaagaaggagatatacatatggctagcatgactggtggacagcaaatgggtcgcggatccatgcaggtgatacccgcgtgcaactcggcagcaataagatcactatgtcctactcccgagtcttttagaaacatgggatggctctctgtcagcgatgcggtctacagcgagttcataggagagttggctacccgcgcttccaatcgaaattactccaacgagttcggcctcatgcaacctatccaggaattcaaggctttcattgaaagcgacccggtggtgcaccaagaatttattgacatgttcgagggcattcaggactctccaaggaattatcaggaactatgtaatatgttcaacgatatctttcgcaaagctcccgtctacggagaccttggccctcccgtttatatgattatggccaaattaatgaacacccgagcgggcttctctgcattcacgagacaaaggttgaaccttcacttcaaaaaacttttcgatacctggggattgttcctgtcttcgaaagattctcgaaatgttcttgtggccgaccagttcgacgacagacattgcggctggttgaacgagcgggccttgtctgctatggttaaacattacaatggacgcgcatttgatgaagtcttcctctgcgataaaaatgccccatactacggcttcaactcttacgacgacttctttaatcgcagatttcgaaaccgagatatcgaccgacctgtagtcggtggagttaacaacaccaccctcatttctgctgcttgcgaatcactttcctacaacgtctcttatgacgtccagtctctcgacactttagttttcaaaggagagacttattcgcttaagcatttgctgaataatgaccctttcaccccacaattcgagcatgggagtattctacaaggattcttgaacgtcaccgcttaccaccgatggcacgcacccgtcaatgggacaatcgtcaaaatcatcaacgttccaggtacctactttgcgcaagccccgagcacgattggcgaccctatcccggataacgattacgacccacctccttaccttaagtctcttgtctacttctctaatattgccgcaaggcaaattatgtttattgaagccgacaacaaggaaattggcctcattttccttgtgttcatcggcatgaccgaaatctcgacatgtgaagccacggtgtccgaaggtcaacacgtcaatcgtggcgatgacttgggaatgttccatttcggtggttcttcgttcgcgcttggtctgaggaaggattgcagggcagagatcgttgaaaagttcaccgaacccggaacagtgatcagaatcaacgaagtcgtcgctgctctaaaggcttagaagcttgcggccgcactcgagtctggtaaagaaaccgctgctgcgaaatttgaacgccagcacatggactcgtctactagaaataattttgtttaactttaagaaggagatatacatatggctagcatgactggtggacagcaaatgggtcgcggatccatggcgttcgatctcaagactgaagacggcctcatcacatatctcactaaacatctttctttggacgtcgacacgagcggagtgaagcgccttagcggaggctttgtcaatgtaacctggcgcattaagctcaatgctccttatcaaggtcatacgagcatcatcctgaagcatgctcagccgcacatgtctacggatgaggattttaagataggtgtagaacgttcggtttacgaataccaggctatcaagctcatgatggccaatcgggaggttctgggaggcgtggatggcatagtttctgtgccagaaggcctgaactacgacttagagaataatgcattgatcatgcaagatgtcgggaagatgaagacccttttagattatgtcaccgccaaaccgccacttgcgacggatatagcccgccttgttgggacagaaattggggggttcgttgccagactccataacataggccgcgagaggcgagacgatcctgagttcaaattcttctctggaaatattgtcggaaggacgacttcagaccagctgtatcaaaccatcatacccaacgcagcgaaatatggcgtcgatgaccccttgctgcctactgtggttaaggaccttgtggacgatgtcatgcacagcgaagagacccttgtcatggcggacctgtggagtggaaatattcttctccagttggaggagggaaacccatcgaagctgcagaagatatatatcctggattgggaactttgcaagtacggcccagcgtcgttggacctgggctatttcttgggtgactgctatttgatatcccgctttcaagacgagcaggtcggtacgacgatgcggcaagcctacttgcaaagctatgcgcgtacgagcaagcattcgatcaactacgccaaagtcactgcaggtattgctgctcatattgtgatgtggaccgactttatgcagtgggggagcgaggaagaaaggataaattttgtgaaaaagggggtagctgcctttcacgacgccaggggcaacaacgacaatggggaaattacgtctaccttactgaaggaatcatccactgcgtaaaagcttgcggccgcactcgagtctggtaaagaaaccgctgctgcgaaatttgaacgccagcacatggactcgtctactagaaataattttgtttaactttaagaaggagatatacatatggctagcatgactggtggacagcaaatgggtcgcggatccatgcatatcagaaatccttaccgtacaccaattgactatcaagcactttcagaggccttccctcccctcaagccatttgtgtctgtcaatgcagatggtaccagttctgttgacctcactatcccagaagcccagagggcgttcacggccgctcttcttcatcgtgacttcgggctcaccatgaccataccagaagaccgtctgtgcccaacagtccccaataggttgaactacgttctgtggattgaagatattttcaactacacgaacaaaaccctcggcctgtcggatgaccgtcctattaaaggcgttgatattggtacaggagcctccgcaatttatcctatgcttgcctgtgctcggttcaaggcatggtctatggttggaacagaggtcgagaggaagtgcattgacacggcccgcctcaatgtcgtcgcgaacaatctccaagaccgtctctcgatattagagacatccattgatggtcctattctcgtccccattttcgaggcgactgaagaatacgaatacgagtttactatgtgtaaccctccattctacgacggtgctgccgatatgcagacttcggatgctgccaaaggatttggatttggcgtgggcgctccccattctggaacagtcatcgaaatgtcgactgagggaggtgaatcggctttcgtcgctcagatggtccgtgagagcttgaagcttcgaacacgatgcagatggtacacgagtaacttgggaaagctgaaatccttgaaagaaatagtggggctgctgaaagaacttgagataagcaactatgccattaacgaatacgttcaggggtccacacgtcgttatgccgttgcgtggtctttcactgatattcaactgcctgaggagctttctcgtccctctaaccccgagctcagctctcttttctagctcgagtctggtaaagaaaccgctgctgcgaaatttgaacgccagcacatggactcgtctactagtcgcagcttaattaacctaaactgctgccaccgctgagcaataactagcataaccccttggggcctctaaacgggtcttgaggggttttttgctagcgaaaggaggagtcgactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagcctaggatcgagatcgatctcgatcccgcgaaat

SEQ ID NO: 29 - DNA, 26 bp, synthetic construct
  source 1..26: mol_type = other DNA
ggtccatatggcactattgttagctg

SEQ ID NO: 30 - DNA, 32 bp, synthetic construct
  source 1..32: mol_type = other DNA
ccgcggccatatggcgagctctagcagcgatg

SEQ ID NO: 31 - DNA, 30 bp, synthetic construct
  source 1..30: mol_type = other DNA
ccgcggcatatggacacgcagaaaccggcg

SEQ ID NO: 32 - DNA, 22 bp, synthetic construct
  source 1..22: mol_type = other DNA
cctctggtgagcgcaagttgtc

SEQ ID NO: 33 - DNA, 21 bp, synthetic construct
  source 1..21: mol_type = other DNA
gaagtggagcgcttgctttgc

SEQ ID NO: 34 - DNA, 25 bp, synthetic construct
  source 1..25: mol_type = other DNA
gctgaacctgaagtatgccgtgaac

SEQ ID NO: 35 - DNA, 26 bp, synthetic construct
  source 1..26: mol_type = other DNA
gtgttgattgaacaaggtgcgaagag

SEQ ID NO: 36 - DNA, 24 bp, synthetic construct
  source 1..24: mol_type = other DNA
gataaagaagacggcaccccgctg

SEQ ID NO: 37 - DNA, 21 bp, synthetic construct
  source 1..21: mol_type = other DNA
cgctttcgctcgagttagctc

SEQ ID NO: 38 - DNA, 21 bp, synthetic construct
  source 1..21: mol_type = other DNA
cgctttcgctcgagttaccag

SEQ ID NO: 39 - DNA, 36 bp, synthetic construct
  source 1..36: mol_type = other DNA
gcggcgcatatgatcgctgtactattctccttcgtc

SEQ ID NO: 40 - DNA, 33 bp, synthetic construct
  source 1..33: mol_type = other DNA
gccgcgctcgagtcagggtccggataccgactg

SEQ ID NO: 41 - DNA, 55 bp, synthetic construct
  source 1..55: mol_type = other DNA
gaaggtctggcgagaagccgccgaccacatggttgacatgccttatgaaactatg

SEQ ID NO: 42 - DNA, 55 bp, synthetic construct
  source 1..55: mol_type = other DNA
catagtttcataaggcatgtcaaccatgtggtcggcggcttctcgccagaccttc

SEQ ID NO: 43 - DNA, 25 bp, synthetic construct
  source 1..25: mol_type = other DNA
gcccgccatatggcgctgctgctgg

SEQ ID NO: 44 - DNA, 61 bp, synthetic construct
  source 1..61: mol_type = other DNA
gcccgccatatggctctgttattagcagttttttactacatcgtttctcgtagagtgaggc

SEQ ID NO: 45 - DNA, 56 bp, synthetic construct
  source 1..56: mol_type = other DNA
gcccgccatatggctaagaaaacgagctctaaagggaagttgccaccagggccgcc

SEQ ID NO: 46 - DNA, 34 bp, synthetic construct
  source 1..34: mol_type = other DNA
gcccgccatatggcgcgtagagtgaggcggtcgc

SEQ ID NO: 47 - DNA, 29 bp, synthetic construct
  source 1..29: mol_type = other DNA
cgctcggctcgagtcagggtccggatacc

SEQ ID NO: 48 - DNA, 40 bp, synthetic construct
  source 1..40: mol_type = other DNA
gcccgccatatggctctgttattagcagtttttattgtgc

SEQ ID NO: 49 - DNA, 51 bp, synthetic construct
  source 1..51: mol_type = other DNA
gcccgccatatggctctgttattagcagttttctactatgcgaacgcgcgc

SEQ ID NO: 50 - DNA, 54 bp, synthetic construct
  source 1..54: mol_type = other DNA
gcccgccatatggctaagaaaacgagctctaaagggaagcttccgcctggcccg

SEQ ID NO: 51 - DNA, 54 bp, synthetic construct; identical to SEQ ID NO: 50
  source 1..54: mol_type = other DNA

SEQ ID NO: 52 - DNA, 25 bp, synthetic construct
  source 1..25: mol_type = other DNA
tctcggctcgagtcagccgctcacg

SEQ ID NO: 53 - DNA, 38 bp, synthetic construct
  source 1..38: mol_type = other DNA
gcccgccatatggctctgttattagcagtttttcaagg

SEQ ID NO: 54 - DNA, 55 bp, synthetic construct
  source 1..55: mol_type = other DNA
gcccgccatatggctctgttattagcagttttttgcgtgtactacgcacactctc

SEQ ID NO: 55 - DNA, 57 bp, synthetic construct
  source 1..57: mol_type = other DNA
gcccgccatatggctaagaaaacgagctctaaagggaagctgcctcctggacccccg

SEQ ID NO: 56 - DNA, 28 bp, synthetic construct
  source 1..28: mol_type = other DNA
gcccgccatatggcccgtcgtgcgcggc

SEQ ID NO: 57 - DNA, 29 bp, synthetic construct
  source 1..29: mol_type = other DNA
gcgtcgctcgagtcacacatcttgcaccg

SEQ ID NO: 58 - DNA, 32 bp, synthetic construct
  source 1..32: mol_type = other DNA
gcccgccatatggcccgtcgtgcgcggcgtgc

SEQ ID NO: 59 - DNA, 42 bp, synthetic construct
  source 1..42: mol_type = other DNA
gcccgccatatggctctgttattagcagtttttattaacctg

SEQ ID NO: 60 - DNA, 58 bp, synthetic construct
  source 1..58: mol_type = other DNA
gcccgccatatggctctgttattagcagttttttattatattgtgagccgccgcattc

SEQ ID NO: 61 - DNA, 57 bp, synthetic construct
  source 1..57: mol_type = other DNA
gcccgccatatggctaagaaaacgagctctaaagggaagctgccacctggcccaccg

SEQ ID NO: 62 - DNA, 32 bp, synthetic construct
  source 1..32: mol_type = other DNA
gcccgccatatggctcgccgcattcgccgcag

SEQ ID NO: 63 - DNA, 28 bp, synthetic construct
  source 1..28: mol_type = other DNA
gctcgcgctcgagtcacaggccgctcag

SEQ ID NO: 64 - DNA, 36 bp, synthetic construct
  source 1..36: mol_type = other DNA
gcccgccatatggctctgttattagcagtttttgcg

SEQ ID NO: 65 - DNA, 62 bp, synthetic construct
  source 1..62: mol_type = other DNA
gcccgccatatggctctgttattagcagtttttggctgcatttattatattaacgcacgtcg

SEQ ID NO: 66 - DNA, 58 bp, synthetic construct
  source 1..58: mol_type = other DNA
gcccgccatatggctaagaaaacgagctctaaagggaagctgcctcctggccctcctg

SEQ ID NO: 67 - DNA, 35 bp, synthetic construct
  source 1..35: mol_type = other DNA
gcccgccatatggccaacgcacgtcgcattaaacg

SEQ ID NO: 68 - DNA, 28 bp, synthetic construct
  source 1..28: mol_type = other DNA
gctccgctcgagtcagcgaaaaaagccg

SEQ ID NO: 69 - DNA, 40 bp, synthetic construct
  source 1..40: mol_type = other DNA
gcccgccatatggcactattgttagctgtatttaggagag

SEQ ID NO: 70 - DNA, 72 bp, synthetic construct
  source 1..72: mol_type = other DNA
gcccgccatatggctaagaaaacgagctctaaagggaagctcccaccaggaccatcgaccacgaaaccggcg

SEQ ID NO: 71 - DNA, 34 bp, synthetic construct
  source 1..34: mol_type = other DNA
gcccgccatatggcacgtgatcaactgttcgccg

SEQ ID NO: 72 - DNA, 52 bp, synthetic construct
  source 1..52: mol_type = other DNA
gcccgccatatggctctgttattagcagttttccgtgatcaactgttcgccg

SEQ ID NO: 73 - DNA, 28 bp, synthetic construct
  source 1..28: mol_type = other DNA
tctcggctcgagttagctccacacgtcc